(19) United States
(12) Patent Application Publication
(10) Pub. No.: US 2005/0192483 A1
(43) Pub. Date: Sep. 1, 2005

(54) Device

(76) Inventors: Frank Bonadio, Bray (IE); John Butler, Deansgrange (IE); Trevor Vaugh, Garbally (IE); Ronan Bernard McManus, Bray (IE); Shane Joseph MacNally, Bray (IE); Alan Reid, Clontarf (IE); Alfred Cuschieri, Denbrae Mill (GB)

Correspondence Address: Finnegan, Henderson, Farabow,;Garrett & Dunner, L.L.P., 1300 I Street, N.W., Washington, DC, 20005-3315, US

(21) Appl. No.: 10/902,440

(22) Filed: Jul. 30, 2004

Related U.S. Application Data
(63) Continuation-in-part of application No. 10/736,234, filed on Dec. 16, 2003.
(63) Continuation-in-part of application No. 10/678,653, filed on Oct. 6, 2003.
Application No. 10/678,653 is a continuation-in-part of application No. 10/133,979, filed on Apr. 29, 2002, now Pat. No. 6,846,287.
Application No. 10/133,979 is a continuation of application No. 09/801,826, filed on Mar. 9, 2001, now abandoned.
Application No. 09/801,826 is a continuation of application No. PCT/IE99/00122, filed on Dec. 1, 1999.
Application No. 10/678,653 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
(63) Continuation-in-part of application No. 10/665,395, filed on Sep. 22, 2003.
Application No. 10/665,395 is a continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
(63) Continuation-in-part of application No. 10/315,233, filed on Dec. 10, 2002.
Application No. 10/315,233 is a continuation of application No. 09/804,552, filed on Mar. 13, 2001, now Pat. No. 6,578,577.
Application No. 09/804,552 is a continuation of application No. PCT/IE99/00123, filed on Dec. 1, 1999.
(60) Provisional application No. 60/433,603, filed on Dec. 16, 2002.
(60) Provisional application No. 60/453,200, filed on Mar. 11, 2003.
(60) Provisional application No. 60/415,780, filed on Oct. 4, 2002.
(60) Provisional application No. 60/428,215, filed on Nov. 22, 2002.
(60) Provisional application No. 60/490,909, filed on Jul. 30, 2003.

(30) Foreign Application Priority Data
Feb. 15, 1999 (IE) 990111
Dec. 1, 1998 (IE) 980997
Dec. 16, 1999 (IE) 991053
Oct. 14, 1999 (IE) 990861
May 24, 1999 (IE) 990416
Feb. 15, 1999 (IE) 990112
Feb. 15, 1999 (IE) 990110
Feb. 15, 1999 (IE) 990108
Feb. 15, 1999 (IE) 990107
Dec. 1, 1998 (IE) 980999
Sep. 19, 2002 (IE) 2002/0754
Feb. 18, 2000 (EP) 00650010

(51) Int. Cl. A61M 29/00

(52) U.S. Cl. 600/208

(57) ABSTRACT

An instrument access port comprises a retractor (811) for retracting the sides of an incision (813) and a valve (829) for sealing around an instrument (814) inserted through the retracted incision (813). The retractor (811) comprises a distal member for insertion into the incision (813), a proximal member for location externally of the incision (813) and a retracting member for extending between the distal member and the proximal member. The valve (829) is coupled to the retractor (811) to define a low profile sealed instrument access port.

### INTRODUCTION

Accessing the abdominal cavity while preserving the abdominal wall as much as possible is the aim of any surgical or exploratory procedure. Retraction devices have been used to this end. A retractor can help to expose an operative site and minimise the incision required to carry out the operation.

Minimally invasive surgery is an evolving surgical method that similarly attempts to reduce the size of incisions required, in many cases dramatically. By using a so-called “keyhole” or cannula, the surgeon can gain access with instruments into the abdominal cavity to carry out an operation through a very small series of holes in the abdominal wall. Unlike in the case of “open surgery”, primary retraction then must be accomplished by lifting the abdominal wall away from the abdominal viscera. This is most often accomplished with the use of gas in a technique known as insufflation.

The use of a cannula to gain access as a means to see inside the abdomen or introduce surgical instruments has existed since the late 19^{th} century. A cannula comprises a rigid tube, which is inserted through the abdominal wall and is held in place by the tension of the abdominal wall itself around the inserted cannula. The tube must accommodate various thicknesses of abdominal wall and extend significantly both inside and outside the abdomen to avoid slipping out of the incision, and thereby causing gas pressure to escape.

The basic construction of a cannula, however, presents significant limitations in carrying out a surgical procedure. Some of these limitations are as follows.
1. A cannula is held in place, and thus prevents the escape of gas, by tissue tension. This tension can vary depending on the way the cannula is introduced or weaken during the operation under normal surgical manipulation.
2. A cannula extends significantly into the abdominal cavity taking up precious space and interfering with other instruments.
3. A cannula restricts the movement of instruments as they are rigid structures.
4. A rigid cannula presents significant limitations on the design of the instrument which must be passed through the cannula.
5. A cannula takes up a significant space outside of the abdomen, shortening the effective length, and therefore reach, of the surgical instrument.

This invention is directed towards providing a surgical device which will address at least some of these problems.

### STATEMENT OF INVENTION

According to the invention there is provided an instrument access port comprising:
a retractor for retracting the sides of an incision;
the retractor comprising a distal member for insertion into the incision, a proximal member for location externally of the incision, and a retracting member for extending between the distal member and the proximal member; and
a valve for sealing around an instrument inserted through a retracted incision;
the valve being coupled to the retractor to define a low profile sealed instrument access port.

In one embodiment of the invention the retractor is configured to retract the sides of a laparoscopic incision. Preferably the retractor is configured to retract the sides of an incision to a diameter substantially equal to a diameter of an instrument to be inserted through the retracted incision. Ideally the retractor is configured to retract the sides of an incision to a diameter substantially equal to a diameter of a laparoscopic instrument to be inserted through the retracted incision.

The retractor may be configured to retract the sides of an incision to a diameter of less than 40 mm, preferably between 3 mm and 35 mm, ideally between 5 mm and 12 mm.

In one case the retracting member is fixedly attached to at least part of the proximal member. Preferably the retracting member is movably coupled to the distal member. Ideally the retracting member is looped around the distal member. Most preferably the retracting member extends between the distal member and the proximal member in a two-layer arrangement. The retracting member may extend distally from the proximal member to the distal member in a first layer and extends proximally from the distal member to the proximal member in a second layer, the first layer being located radially inwardly of the second layer.

In one case the retractor member comprises a sleeve. The distal member may comprise a ring. The proximal member may comprise a ring arrangement. Preferably the proximal member comprises an inner ring part and an outer ring part. Ideally at least part of the retracting member is movably received between the inner ring part and the outer ring part.

In a preferred embodiment the valve is configured to seal around a laparoscopic instrument. Ideally the valve is configured to seal around an instrument having a diameter of less than 40 mm. Most preferably the valve is configured to seal around an instrument having a diameter of between 3 mm and 35 mm. In a particularly preferred case the valve is configured to seal around an instrument having a diameter of between 5 mm and 12 mm.

The valve in one case comprises at least one sealing valve. Preferably the valve comprises a first sealing valve and a second sealing valve. Ideally the first sealing valve is located distally of the second sealing valve.

The sealing valve may comprise an iris valve. The sealing valve may comprise a lip seal. The sealing valve may comprise a duck-bill valve. Preferably the sealing valve is biased towards a closed, sealing configuration. Ideally the sealing valve comprises a biasing element to bias the sealing valve towards the closed, sealing configuration. The biasing element may comprises a coiled spring.

In a further embodiment the port comprises a coupling element for coupling at least part of the valve to the retractor. The coupling element may extend between the valve and the retractor to couple at least part of the valve to the retractor. In one case the coupling element is substantially flexible to accommodate movement of the valve relative to the retractor while maintaining the coupling. Ideally the coupling element comprises a sleeve.

The coupling element comprises in one case a proximally extending portion of the retracting member.

The valve may be engagable with the retractor to couple at least part of the valve to the retractor. Preferably the valve is engagable with the retractor in a snap-fit manner to couple at least part of the valve to the retractor. In one case the valve and the retractor comprise corresponding inter-engagement parts. Ideally the inter-engagement parts comprise a male projecting part on one of the valve or the retractor and a corresponding female recess part on the other of the retractor or the valve.

At least part of the valve may be engagable with at least part of the proximal member of the retractor. Preferably at least part of the valve is engagable with the outer ring part of the retractor.

Preferably the valve is sized for effecting a gas-tight seal with an instrument no larger than a laparoscopic instrument.

In another aspect the invention provides a cannula comprising:
a proximal instrument insertion portion having a seal for sealingly engaging with an instrument shaft; and
a distal tubular portion defining an access channel for extension of an instrument therethrough;
the proximal portion being movably coupled to the distal portion to facilitate relative movement between the proximal portion and the distal portion to accommodate lateral movement of an instrument passing therethrough whilst maintaining sealing engagement between the seal and an instrument shaft.

In one embodiment the cannula comprises a flexible coupling portion to movably couple the proximal portion to the distal portion. Preferably the coupling portion is substantially tubular. Ideally a longitudinal axis of the coupling portion is substantially parallel to a longitudinal axis of the distal portion. The coupling portion may be concertinaed along at least part of the length of the coupling portion. Most preferably the coupling portion comprises a sheath.

The seal may be provided at a proximal end of the proximal portion. Ideally the proximal portion comprises a proximal opening through which an instrument may be inserted into the proximal portion, and the seal is provided at the proximal opening.

In one case the seal comprises a lip seal.

According to a further aspect of the invention, there is provided a cannula comprising:
a proximal instrument insertion portion;
a distal tubular portion defining an access channel for extension of an instrument therethrough; and
a seal for sealingly engaging with an instrument shaft;
the seal being movably coupled to the proximal portion to accommodate lateral movement of an instrument passing therethrough while maintaining sealing engagement between the seal and an instrument shaft.

In one embodiment the seal is located externally of the proximal portion. The seal may be located proximally of a proximal end of the proximal portion. Ideally the proximal portion comprises a proximal opening through which an instrument may be inserted into the proximal portion, and the seal is located proximally of the proximal opening.

In one case the seal comprises a lip seal.

In another embodiment the cannula comprises a flexible coupling portion to movably couple the seal to the proximal portion. Preferably the coupling portion is substantially tubular. Ideally a longitudinal axis of the coupling portion is substantially parallel to a longitudinal axis of the proximal portion. Most preferably the coupling portion is concertinaed along at least part of the length of the coupling portion. The coupling portion may comprise a sheath.

In a further aspect, the invention provides a method of accessing a wound interior with an instrument, the method comprising the steps of:
retracting the sides of an incision;
sealing around an instrument; and
sealingly inserting the instrument through the retracted incision to access the wound interior.

In one embodiment the incision is a laparoscopic incision. Preferably the sides of the incision are retracted to a diameter of less than 40 mm. Ideally the sides of the incision are retracted to a diameter of between 3 mm and 35 mm. Most preferably the sides of the incision are retracted to a diameter of between 5 mm and 12 mm.

The sides of the incision may be retracted to a diameter substantially equal to a diameter of the instrument.

Preferably the instrument is a laparoscopic instrument. The instrument may have a diameter of less than 40 mm. Ideally the instrument has a diameter of between 3 mm and 35 mm. Most preferably the instrument has a diameter of between 5 mm and 12 mm.

In one case the method comprises the steps of:
opening a seal to extend the instrument therethrough; and
closing the seal around the instrument to seal around the instrument.
The seal may be opened by inserting the instrument through the seal. The seal may be opened before extending the instrument through the seal.

The method preferably comprises the step of creating the incision.

In one case the method comprises the step of mounting a retractor in the incision. Ideally the method comprises the step of coupling a seal to a retractor. Most preferably the seal is coupled to the retractor by engaging the seal is coupled to the retractor by engaging the seal with the retractor.

According to the invention there is provided a wound retractor comprising:
a retracting member for insertion into a wound opening; and
a proximal member for location externally of a wound opening;
the proximal member being movable relative to the retracting member to shorten the axial extent of the retracting member to laterally retract a wound opening.

In one embodiment the proximal member comprises an annular ring means.

In one case the annular ring means comprises an inner ring and an outer ring between which the retracting member may be lead. One of the rings may define a projection for location in a complimentary recess of the outer ring with the retracting member located therebetween. The projection may be a relatively tight fit in the recess to grip the retracting member therebetween. In one arrangement the projection is locatable in the recess in a snap-fit manner.

In one embodiment the inner ring defines the projection and the outer ring defines the recess.

Alternatively the outer ring defines the projection and the inner ring defines the recess.

In one embodiment the proximal member comprises one or more valves to facilitate sealed access of an object through the proximal member.

In an aspect of the invention the retractor comprises a distal member coupled to a distal end of the retracting member. The distal member may comprise an O-ring. Alternatively the distal member comprises an annular disc. The distal member may be of a resilient material.

In one embodiment the retracting member is flared distally outwardly.

In one aspect the retractor comprises means to seal a retracted wound opening. The sealing means may be provided externally of a wound opening.

Typically, the sealing means is mountable to the proximal member. The sealing means may comprise a cap.

In one embodiment the sealing means comprises one or more valves to facilitate sealed access of an object through the sealing means.

In one arrangement the retracting member comprises a sleeve to line a wound opening.

The invention also provides a method of retracting a wound opening, the method comprising the steps of:
providing a wound retractor comprising a retracting member, and a proximal member;
inserting the retracting member into a wound opening;
locating the proximal member externally of the wound opening; and
moving the proximal member relative to the retracting member to shorten the axial extent of the retracting member to laterally retract the wound opening.

In one embodiment the retracting member comprises a proximal portion located proximally of the proximal member and a distal portion located distally of the proximal member, and the method comprises the step of decoupling the proximal portion from the distal portion after retraction of the wound opening.

The proximal portion may be decoupled from the distal portion by a cutting action.

In one arrangement the proximal member comprises an inner ring and an outer ring, and the method comprises the step of snap-fitting the inner ring relative to the outer ring to grip the retracting member therebetween. The inner ring may be snap-fitted relative to the outer ring after retraction of the wound opening.

In one embodiment the step of snap-fitting the inner ring relative to the outer ring decouples the proximal portion of the retracting member from the distal portion.

In another aspect the method comprises the step of mounting the retracting member to an obturator, and the obturator is inserted into the wound opening to insert the retracting member into the wound opening.

Typically, the method comprises the step of sealing the retracted wound opening.

According to the invention there is provided a medical device comprising:
a retractor member comprising a distal portion for insertion through an incision made in a patient, and a proximal portion for extending from the incision and outside of the patient;
a distal member associated with the distal portion of the retractor member;
a proximal member associated with the proximal portion of the retractor member;
the retractor member being axially movable relative to the distal member to draw the proximal and distal members towards one another thereby shortening the axial extent of the retractor member between the proximal and distal members.

In one embodiment the retractor member comprises a sleeve member. The sleeve member preferably extends around the distal member.

In one embodiment the distal member is a ring member such as a resilient ring member, for example, an O-ring.

In one embodiment the proximal member is connected to the retractor member. The proximal member may be a ring member.

In one embodiment the sleeve member is of a pliable material.

In one arrangement the sleeve extends from the proximal member, around the distal member and has a return section outside of the proximal member.

The return section may have a handle member such as a ring member.

In one embodiment the device comprises a guide member.

The retractor member may extend between the guide member and the proximal member.

The guide member may comprise a receiver for the proximal member.

The guide member may comprise a guide ring-receiving member.

The sleeve return section may be configured to provide an integral valve member. In this case the sleeve return section may be twisted to provide an iris valve.

In another embodiment the sleeve return section is mounted to the guide member.

The sleeve return section may be extended into the opening defined by the sleeve member.

The device may comprise a lock for locking the guide member to the proximal member. Typically the guide member is engagable with the proximal member to provide the lock.

The guide member may be an interference fit with the proximal member.

In one embodiment of the invention the device includes a valve, such as an iris-type valve.

In one embodiment the device comprises a biassing member for biassing the valve into a desired position such as the closed position.

In one arrangement the device comprises a guide member located proximally of the proximal member and a biassing means is provided between the proximal member and the guide member. The biassing means may comprise a spring such as a coil spring.

In one embodiment a sleeve member extends between the proximal member and the guide member and the biassing means is located around the sleeve. The sleeve member may be an extension of the retractor member.

In one embodiment the device comprises a release member for releasing the device from an incision. The release member may comprise an elongate member such as a pull ribbon or string extending from a distal end of the device.

The release member may extend from the distal member.

In one embodiment the valve is located or locatable proximal of the proximal member. A pliable material may be provided between the valve and the proximal member. The pliable material may comprise a proximal extension of the retractor member.

In one embodiment the pliable material comprises a sleeve section.

In another embodiment the valve is a lip seal.

The invention also provides a method for retracting an incision comprising the steps of:
providing a device comprising a retractor member having a distal portion and a proximal portion, a distal member associated with the distal portion and a proximal portion associated with the proximal portion;
inserting the distal member and the distal portion of the retractor member through an incision made in a patient; and
pulling the retractor member axially relative to the distal member to draw the proximal and distal members towards one another thereby shortening the axial extent of the retractor member between the proximal and distal members and retracting the incision.

According to the invention there is provided an access port comprising:
a mounting element;
a sleeve of pliable material mounted to the mounting element, the sleeve being twisted to define a normally closed access opening;
the sleeve being movable on insertion of an object such as an instrument or a surgeon's hand to open the access opening whilst maintaining sealing engagement with the object.

The mounting element may comprise a first ring element and a second ring element and the sleeve extends between the ring elements.

A biasing means to bias the sleeve to close the access opening may be provided.

The biasing means may be provided by pre-tensioning the sleeve to close the access opening.

In one embodiment the device comprises a spring element to bias the sleeve to close the access opening.

The spring element may extend between the first and second ring elements.

In one embodiment the spring element has opposite ends and at least one of the ends is attached to a ring element.

The invention also provides an access port comprising a device of the invention.

According to one aspect the invention provides an assembly comprising a retractor and a device of the invention. The access port may be releasably mountable to the retractor.

The access port may be alternatively mounted to the retractor.

The invention also provides a method of performing surgery comprising the steps of:
providing a device of the invention;
inserting an object such as an instrument or a hand into the device against the biasing of the sleeve whilst maintaining sealing engagement between the sleeve and the object.

The invention further provides a method of performing a surgical procedure comprising the steps of providing a device of the invention and inserting an object into the device against the biasing of the sleeve whilst maintaining sealing engagement between the sleeve and the object.

In one aspect the invention provides a medical device comprising a retractor member comprising a distal portion for insertion through an incision made in a patient, and a proximal portion for extending from the incision and outside of the patient;
a distal member associated with the distal portion of the retractor member;
a proximal member associated with the proximal portion of the retractor member;
the retractor member being axially movable relative to the distal member to draw the proximal and distal members towards one another thereby shortening the axial extent of the retractor member between the proximal and distal members.

In one embodiment the retractor member comprises a sleeve member. The sleeve member preferably extends around the distal member.

In one embodiment the distal member is a ring member such as a resilient ring member, for example, an O-ring.

In one embodiment the proximal member is connected to the retractor member. The proximal member may be a ring member.

In one embodiment the sleeve member is of a pliable material.

In one arrangement the sleeve extends from the proximal member, around the distal member and has a return section outside of the proximal member.

The return section may have a handle member such as a ring member.

In one embodiment the device comprises a guide member.

The retractor member may extend between the guide member and the proximal member.

The guide member may comprise a receiver for the proximal member.

The guide member may comprise a guide ring-receiving member.

The sleeve return section may be configured to provide an integral valve member. In this case the sleeve return section may be twisted to provide an iris valve.

In another embodiment the sleeve return section is mounted to the guide member.

The sleeve return section may be extended into the opening defined by the sleeve member.

The device may comprise a lock for locking the guide member to the proximal member. Typically the guide member is engagable with the proximal member to provide the lock.

The guide member may be an interference fit with the proximal member.

In one embodiment of the invention the device includes a valve, such as an iris-type valve.

In one embodiment the device comprises a biasing member for biasing the valve into a desired position such as the closed position.

In one arrangement the device comprises a guide member located proximally of the proximal member and a biasing means is provided between the proximal member and the guide member. The biasing means may comprise a spring such as a coil spring.

In one embodiment a sleeve member extends between the proximal member and the guide member and the biasing means is located around the sleeve. The sleeve member may be an extension of the retractor member.

In one embodiment the device comprises a release member for releasing the device from an incision. The release member may comprise an elongate member such as a pull ribbon or string extending from a distal end of the device.

The release member may extend from the distal member.

In one embodiment the valve is located or locatable proximal of the proximal member. A pliable material may be provided between the valve and the proximal member. The pliable material may comprise a proximal extension of the retractor member.

In one embodiment the pliable material comprises a sleeve section.

In another embodiment the valve is a lip seal.

The invention also provides a method for retracting an incision comprising the steps of:
providing a device comprising a retractor member having a distal portion and a proximal portion, a distal member associated with the distal portion and a proximal member associated with the proximal portion;
inserting the distal member and the distal portion of the retractor member through an incision made in a patient; and
pulling the retractor member axially relative to the distal member to draw the proximal and distal members towards one another thereby shortening the axial extent of the retractor member between the proximal and distal members and retracting the incision.

The invention provides an access device for an incision comprising a retractor for the incision and a valve coupled to the retractor.

The valve may be flexibly coupled to the retractor.

The invention also provides an introduction tool for introducing a distal ring of a retractor through an abdominal wall.

### BRIEF DESCRIPTION OF THE DRAWINGS

The invention will be more clearly understood from the following description of some embodiments thereof, given by way of example only, with reference to the accompanying drawings, in which:

Fig. A is a cross sectional view of an access port of the invention mounted in an incision;

Fig. B is a cross sectional view of the port of Fig. 1 with an instrument inserted;

Fig. C is a view similar to Fig. B;

Fig. C¹ is a view comparable with Fig. C of a conventional cannula with the same instrument in situ;

Fig. D is a cross-sectional, side view of a wound retractor according to the invention, in use;

Fig. E is a perspective view of the retractor of Fig. 1 being inserted into a wound opening;

Figs. F to H, K and L are cross-sectional, side views of the wound opening being retracted using the retractor of Fig. D;

Fig. I is a plan view of the retractor and the wound opening of Fig. H;

Fig. K is a plan view of the retractor and the wound opening of Fig. K;

Figs. M and N are views similar to Figs. H and K of a wound opening being retracted in an alternative manner using the retractor of Fig. D;

Figs. O and P are cross-sectional, side views of a wound opening being retracted using the retractor of Fig. D and an obturator;

Figs. Q and R are cross-sectional, side views of a wound opening being retracted using the retractor and the obturator of Figs. O and P and a pusher;

Fig. S is a cross-sectional, side view of the retractor of Fig. D and a sealing cap;

Figs. T and V are perspective views of a distal end of other wound retractors according to the invention;

Figs. W to Y are perspective views of an inner ring part of other wound retractors according to the invention;

Fig. Z is a cross-sectional, side view of another wound retractor according to the invention;

FIG. 1 is a perspective view of a retractor according to the invention;

FIG. 2 is a cross sectional view of the device of FIG. 1;

FIGS. 3 and 4 are perspective views illustrating the formation of the device of FIGS. 1 and 2;

FIGS. 5 and 6 are cross sectional views of FIGS. 3 and 4 respectively;

FIGS. 7 and 8 are perspective views illustrating the use of the device;

FIGS. 9 and 10 are cross sectional views illustrating the method of use of the device;

FIG. 11 is a cross sectional view of another device according to the invention in a configuration ready for use;

FIG. 12 is a perspective view of the device of FIG. 11 with a distal portion inserted through an incision;

FIG. 13 is a cross sectional view of the device of FIG. 11 with a distal portion inserted through an incision;

FIG. 14 is a cross sectional view of the device of FIG. 11 in use with an incision retracted;

FIG. 15 is a perspective view of the device in the configuration of FIG. 14;

FIG. 16 is a perspective view of the device in situ with an excess sleeve portion being removed;

FIG. 17 is a cross sectional view of the device in situ with an excess sleeve portion extending back into the incision;

FIG. 18 is a perspective view of the device in situ with a excess sleeve portion being twisted;

FIG. 19 is a perspective view similar to FIG. 18 with the excess sleeve portion further twisted to provide an iris valve;

FIG. 20 is a cross sectional view of another device according to the invention in situ;

FIG. 21 is a cross sectional view of the device of FIG. 20 with an excess sleeve portion mounted to a guide member;

FIG. 22 is a cross sectional view of the device of FIG. 21 with the excess sleeve portion inflated to provide an integral everting access part;

FIG. 23 is a perspective view of another retractor according to the invention incorporating a release device;

FIG. 24 is a cross sectional view of the retractor of FIG. 23;

FIG. 25 is a perspective view illustrating the formation of the device of FIG. 23;

FIG. 26 is a cross sectional view of the device in the configuration of FIG. 25;

FIG. 27 is a cross sectional view of the retractor of FIGS. 23 to 26, in use;

FIG. 28 is a cross sectional view of the retractor of FIGS. 23 to 27 illustrating the operation of a release device;

FIG. 29 is a perspective view of another device according to the invention in an insertion configuration;

FIG. 30 is a perspective view of the device of FIG. 29 in position in an incision;

FIG. 31 is another perspective view of the device of FIG. 30 in another configuration;

FIG. 32 is another view of the device of FIG. 31 with an outer portion severed and a valve being formed;

FIG. 33 is a view of the device of FIG. 32 with the valve closed;

FIG. 34 is a perspective view of another device similar to the device of FIGS. 29 to 33 with a valve closed;

FIG. 35 is a cross sectional view of the device of FIG. 34;

FIG. 36 is a perspective view of another device similar to the device of FIGS. 29 to 33 incorporating a biasing means in an inserted configuration;

FIG. 37 is another perspective view of the device of FIG. 36 in a retracting configuration;

FIG. 38 is a perspective view of the device of FIG. 37 in another configuration and excess sleeve being removed;

FIG. 39 is a perspective view of the device of FIG. 38 with a valve closed;

FIG. 40 is a perspective view of the device of FIG. 39 with a valve partially open;

FIG. 41 is a perspective view of the device of FIG. 39 with an object inserted through the valve;

FIG. 42 is a perspective view of another device according to the invention;

FIG. 43 is a cross sectional view of the device of FIG. 42 in position in an incision;

FIG. 44 is a cross sectional view of the device of FIG. 43 with an object extending therethrough;

FIG. 45 is a cross sectional view similar to FIG. 44 with an object offset from a longitudinal axis of the device;

FIG. 46 is a cross sectional view of another device according to the invention on insertion into an incision;

FIG. 47 is a cross sectional view of the device of FIG. 46 with an incision retracted;

FIGS. 48 and 49 are cross sectional views of the device of FIG. 47 showing the formation of an iris valve;

FIG. 49(a) is a cross sectional view of another device of the invention;

FIG. 49(b) is a plan view of another hand access device in a closed position;

FIG. 49(c) is a plan view of the device of FIG. 49(b) in an opened position;

FIG. 49(d) is a plan view showing the opening of the device of FIGS. 49(b) and 49(c);

FIGS. 49(e) and (f) are, respectively, plan and side views of the hand access device of FIG. 49(b) in a closed position;

FIGS. 49(g) and (h) are views similar to FIGS. 49(e) and (f) with the device in an open position;

FIG. 49(i) is a cross sectional view of a hand access device with an arm in position;

FIG. 49(j) is a view of a device similar to FIG. 49(i) with a lip seal; and

FIG. 49(k) is a view of a device similar to FIG. 49(i) with another lip seal.

FIG. 50 is a perspective view of a hand access device according to the invention in use;

FIG. 51 is a perspective view of the device of FIG. 50 in use with a hand being pushed through the device;

FIG. 52 to 54 are side cross sectional views of the device of FIGS. 50 and 51 with a surgeon's hand being progressively inserted through the device;

FIGS. 54(a) to 54(d) are views illustrating an assembly of a hand access device;

FIGS. 55(a) to (c) are, respectively, plan, side and side cross sectional views of the device of FIGS. 50 to 54 in a closed configuration;

FIGS. 56(a) to (c) are views similar to FIG. 55 with the device partially open;

FIGS. 57 (a) to (c) are views similar to FIG. 55 with the device closed;

FIG. 58(a) to 60(c) are views similar to FIGS. 55(a) to 57(c) of another device according to the invention;

FIG. 61 is a cross sectional view of the device of FIGS. 50 to 57(c) mounted on a retractor;

FIG. 62 is a cross sectional view of the device of FIGS. 50 to 57(c) being mounted on another retractor.

FIG. 63 is a cross sectional view of the device, fully assembled to the retractor of FIG. 62.

FIG. 64 is a perspective view of another hand access device;

FIG. 65 is a perspective view of the device of FIG. 64 with a hand being inserted;

FIGS. 66 and 67 are perspective views of hand access devices;

FIG. 68 is a cross sectional view of the hand access device of FIGS. 64 and 65 mounted on a retractor with excess retractor sleeve and a lip seal;

FIG. 69 is a cross sectional view of the device of FIG. 68 with an arm in place;

FIG. 70 is a view of another arrangement similar to that of FIGS. 68 and 69;

FIG. 71 is an exploded perspective view of an assembly of the invention comprising a retractor and an iris valve;

FIG. 72 is a cross sectional view of the device of FIG. 71 assembled and in position in an incision;

FIG. 73 is a top plan view of the device of FIG. 72 with the iris closed;

FIG. 74 is a reverse plan view of the device of FIG. 72 in the configuration of FIG. 73;

FIG. 75 is a top plan view of the device of FIG. 72 with the iris open;

FIG. 76 is a reverse plan view of the device of FIG. 72 in the configuration of FIG. 75;

FIG. 77 is an exploded perspective view of a valve of the invention;

FIG. 78 is a top plan view of the assembled valve of FIG. 77 in a closed configuration;

FIG. 79 is a cross sectional view of the valve of FIG. 78;

FIG. 80 is a top plan view of the assembled valve of FIG. 77 in an open configuration to receive an object;

FIG. 81 is a cross sectional view of the valve of FIG. 80;

FIGS. 82 and 83 are respectively plan and cross sectional views of the closed valve of FIGS. 78 and 79;

FIG. 84 is an enlarged cross sectional view of the valve of FIG. 77;

FIG. 85 is a cross sectional view of an access port comprising a retractor base, a valve mounted to the base and a secondary seal for an object such as an instrument;

FIGS. 86 to 88 are cross sectional views of the port of FIG. 85 showing the insertion of an instrument;

FIG. 89 is a cross sectional view of another access port;

FIG. 90 is a cross sectional view of the port of FIG. 89 with an instrument in position;

FIG. 91 is a cross sectional view of a further access port;

FIG. 92 is a cross sectional view of the port of FIG. 91 with an instrument in position;

FIG. 93 is a cross sectional view of another access port;

FIG. 94 is a cross sectional view of the port of FIG. 93 with an instrument in position;

FIG. 95 is a perspective view of another valve and an associated mounting ring;

FIG. 96 is a cross-sectional view illustrating mounting of the valve of FIG. 95 on a retractor;

FIG. 97 is a cross sectional view of the valve of FIG. 95 mounted on a retractor;

FIG. 98 is a perspective view of a mounting ring for a valve;

FIG. 99 is a top perspective view of a cap and valve for use with the mounting ring of FIG. 98;

FIG. 100 is an underneath perspective view of the cap and valve of FIG. 99;

FIGS. 101 to 104 are cross sectional views of an access port incorporating the mounting ring of FIG. 98 and the cap and valve of FIGS. 99 and 100;

FIGS. 105 to 108 are cross sectional views of another access port;

FIGS. 109 and 110 are cross sectional views of a further access port;

FIGS. 111 [unused];

FIGS. 112 and 113 are cross sectional views of another access port;

FIGS. 114 to 116 are cross sectional views of a further access port;

FIGS. 117 to 120 are cross sectional views of another access port;

FIG. 121 is a view of an introducer tool according to the invention;

FIGS. 122 to 124 are views of a retractor distal ring;

FIGS. 125 to 127 are views of another introducer tool;

FIGS. 128 and 129 are views of a further introducer tool;

FIGS. 130 to 134 are cross sectional views of the tool of FIGS. 128 and 129, in use;

FIGS. 135 and 136 are cross sectional views of another introducer tool, in use;

FIGS. 137 to 140 are cross sectional views of an introducer tool, in use;

FIGS. 141 to 144 are cross sectional views of another introducer tool, in use;

FIGS. 145 and 146 are cross sectional views of a cannula of the invention, in use; and

FIGS. 147 to 149 are cross sectional views of another cannula of the invention, in use.

### DETAILED DESCRIPTION

Referring to Figs. A to C there is illustrated an access device of the invention for an incision a, for example in an abdominal wall b. The access device comprises a retractor c for retracting the incision a, and a valve d coupled to the retractor c. The valve d may be flexibly coupled to the retractor c by a sleeve e of flexible material. The construction of the various components and their attributes will be explained in detail below. In general, the access port is in this case used as a substitute for a conventional rigid tubular cannula x, which is illustrated in Fig. C¹.

The access port of the invention may be used to provide access to the abdominal cavity by an instrument f, which in this case has an operating element g, such as a surgical stapler, mounted at the distal end of a flexible shaft h.

It will be noted that the retractor c has a very low profile and is positively retained in the incision a against pull-out forces. Because of the low profile the flexible shaft h of the instrument f can begin bending immediately after entering the abdominal cavity, as illustrated in Figs B and C. The amount of free space required to manipulate the instrument f is minimised. This is in contrast to a conventional cannula x of Fig. C¹, in which the rigid tube of the cannula x is extended significantly into the abdomen to ensure that it remains anchored in the abdomen, otherwise gas pressure may cause it to become dislodged. Because of this cannula length extending into the abdomen, the shaft h of the instrument f cannot be steered until the steerable section has exited the cannula x. Thus, there are severe limitations on the use of such instruments using a conventional cannula x. These problems are overcome using the access port of the invention.

Referring to Figs. D to S, there is illustrated a wound retractor 1 according to the invention. The retractor 1 comprises a proximal member 2 for location, in use, externally of a wound opening 3, a retracting member 4 for insertion into the wound opening 3, and a distal member 5 coupled to a distal end of the retracting member 4.

In this case, the retracting member 4 is provided in the form of a sleeve of flexible, polymeric film material which lines the sides of the wound opening 3 when the retractor 1 is in use (Fig. D). The distal member 5 in this case comprises a resilient O-ring.

The proximal member 2 is provided, in this case, in the form of an annular ring means having an inner ring 6 and an outer ring 7 with the retracting member 4 lead between the rings 6, 7. The inner ring 6 has a circular cross-section and the outer ring 7 defines a “C”-shaped recess. In this manner a projecting portion of the inner ring 6 may be located in a snap-fit manner in the complimentary recess of the outer ring 7. The inner ring 6 is configured to be a relatively tight fit in the recess of the outer ring 7 to securely grip the retracting member 4 between the two rings 6, 7.

In use, a relatively small incision 8 is made in an abdominal wall 9 to form the wound opening 3. A typical length for the incision 8 is in the range of from 12 mm to 30 mm. The resilient distal O-ring 5 is then manipulated into an elongate, oblong shape by squeezing the distal O-ring 5 to facilitate insertion of the distal O-ring 5 through the wound opening 3 (Fig. E), until the distal O-ring 5 is fully located within the abdominal cavity 10 and the sleeve 4 lines the wound opening 3 (Fig. F). The sleeve 4 is then pulled upwardly to cause the distal O-ring 5 to engage with the internal surface of the abdominal wall 9 (Fig. G).

Next the proximal member 2 is threaded over the sleeve 4 with the sleeve 4 passing between the inner ring 6 and the outer ring 7 and the inner ring etc. The proximal member 2 is then moved downwardly relative to the sleeve 4 by pulling the sleeve 4 taut upwardly and pushing the proximal member 2 downwardly (Figs. H and I). This action of moving the proximal member 2 relative to the sleeve 4 shortens the axial extent of the portion of the sleeve 4 which lines the wound opening 3, and thereby results in lateral retraction of the wound opening 3, as illustrated in Figs. J and K.

The tight-fit arrangement of the inner ring 6 in the recess of the outer ring 7 ensures that the sleeve 4 is securely gripped between the rings 6, 7. Thus the proximal member 2 acts as a lock to maintain the wound opening 3 in the retracted configuration illustrated in Figs. J and K.

The portion of the sleeve 4 proximally of the rings 6, 7 is thereafter surplus to requirements and may be removed, for example by cutting it away (Fig. L).

By engaging the internal surface of the abdominal wall 9, the distal O-ring 5 acts as an anchor to maintain the retractor 1 in position in the wound opening 3, during use.

An alternative method of using the wound retractor 1 to retract the wound opening 3 is illustrated in Figs. M and N. In this case, the inner ring 6 and the outer ring 7 are moved downwardly relative to the sleeve 4 before the inner ring 6 is snap-fitted into position in the recess of the outer ring 7. The inner ring 6 is located above the outer ring 7.

The inner ring 6 is pushed downwardly, which causes the outer ring 7 to move downwardly also, while pulling the sleeve 4 taut upwardly until the outer ring 7 engages the external surface of the abdominal wall 9. Further pushing of the inner ring 6 downwardly then causes the inner ring 6 to snap into position in the recess of the outer ring 7 securely gripping the sleeve 4 between the rings 6, 7. The action of the inner ring 6 snapping into position in the recess of the outer ring 7 may be configured to cut the sleeve 4 for subsequent removal of the surplus proximal portion of the sleeve 4.

Referring to Figs. O to R there is illustrated another method of using the wound retractor 1. In this case the retractor 1 is mounted to a blunt obturator 11 before insertion into the wound opening 3. The obturator 11 and the retractor 1 are then inserted together through the wound opening 3 until the distal O-ring 5 is fully located within the abdominal cavity 10 and the sleeve 4 lines the wound opening 3 (Fig. O).

The distal O-ring 5 is engaged with the internal surface of the abdominal wall 9, and the proximal member 2 is moved downwardly relative to the sleeve 4 (Fig. P), in a manner similar to that described previously with reference to Figs. G to K. The obturator 11 may then be removed from the wound opening 3. The proximal member 2 acts as a lock thereafter to maintain the wound opening 3 in the retracted configuration.

It has been found that the use of the obturator 11 may assist in deployment of the wound retractor 1. In particular, retraction of the wound opening 3 by means of the sleeve 4 during the set-up procedure is not required when the obturator 11 is employed.

A sharp obturator could alternatively be employed in a similar manner to that described previously with reference to Figs. O and P. A sharp obturator has the additional advantage that the initial incision 8 in the abdominal wall 9 could be made using the sharp obturator.

Figs. Q and R illustrate a further method of retracting the wound opening 3 using the wound retractor 1, which is similar to the method described previously with reference to Figs. O and P.

In this case, the retractor 1 is mounted to the obturator 11 before the inner ring 6 is snap-fitted into position in the recess of the outer ring 7. A tubular pusher 12 is slidably mounted around the obturator 11 for engagement with the inner ring 6.

By pushing on the pusher 12 downwardly while pulling the sleeve 4 taut upwardly, the rings 6, 7 are moved downwardly until the outer ring 7 engages the external surface of the abdominal wall 9. Further pushing of the pusher 12 downwardly then causes the inner ring 6 to snap into position in the recess of the outer ring 7, and simultaneously causes cutting of the sleeve 4.

The sleeve 4 is thus securely gripped between the rings 6, 7 to maintain the wound opening 3 in the retracted configuration. Also the surplus proximal portion of the sleeve 4 which has been cut away may be removed.

The retractor 1 may include means to seal the retracted wound opening 3. For example, Fig. S illustrates a sealing cap 13 releasably mounted to the proximal member 2 externally of the wound opening 3. The cap 13 may be temporarily mounted to the proximal member 2 to maintain a gas-tight seal of the retracted wound opening 3, for example to maintain pneumoperitoneum within the abdominal cavity 10. If it is desired to access the abdominal cavity 10, and/or to remove matter from within the abdominal cavity 10, the cap 13 can be quickly and easily removed to reveal the retracted wound opening 3.

It will be appreciated that various other sealing means may alternatively be provided with the wound retractor 1. For example, one or more valves may be included to facilitate sealed access of an object, such as an instrument, through the retracted wound opening 3.

The distal end of the sleeve 4 may be flared distally outwardly towards the distal O-ring 20, as illustrated in the wound retractor 25 of Fig. T. This arrangement enhances the anchoring of the retractor 25 in position in the wound opening 3 with less risk of the distal O-ring 20 being pulled up through the wound opening 3, during use.

A variety of different configurations are possible for the distal member of the wound retractor within the scope of this invention. For example, the distal member may be a standard O-ring 21, as illustrated in the wound retractor 26 of Fig. U, or the distal member may be provided in the form of a flexible, annular disc 22, as illustrated in the wound retractor 27 of Fig. V. It has been found that the disc 22 provides enhanced anchoring of the retractor 27 in position in the wound opening 3, during use.

In addition, a variety of different configurations are possible for the proximal member of the wound retractor within the scope of the invention. For example, the inner ring of the proximal member may be provided in the form of a standard O-ring 30, as illustrated in Fig. W. Alternatively one or more valves, such as a lip seal 32, may be provided as part of the inner ring 31, as illustrated in Fig. X to facilitate sealed access of an object, such as an instrument, through the proximal member. As a further alternative, the proximal member may comprise a closed cap 33 (Fig. Y) to completely seal the retracted wound opening 3, for example, to maintain pneumoperitoneum in the abdominal cavity 10.

It will be appreciated that the configuration of the proximal member 2 may be reversed. For example, an inner ring 41 may define a “C”-shaped recess and an outer ring 40 may have a circular cross-section, as illustrated in Fig. Z.

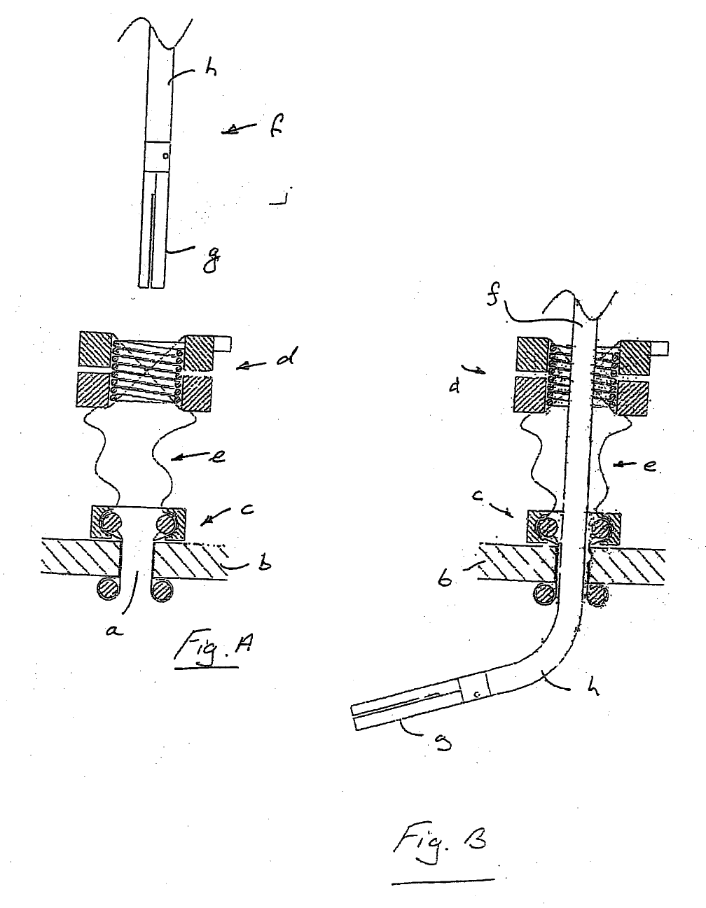

Referring to FIGS. 1 to 10 thereof there is illustrated a medical device 1 comprising a retractor member provided by a sleeve 2, a distal member provided by a distal ring 3 of resilient material such as an O-ring and a proximal member provided by a proximal ring 4 which may also be an O-ring.

The sleeve 2 is of any suitable material such as of pliable plastics film material and comprises a distal portion 5 for insertion through an incision 6, in this case made in a patient's abdomen 7, and a proximal portion 8 for extending from the incision 6 and outside of the patient.

In this case the distal ring 3 is not fixed to the sleeve 2 but rather the sleeve is led around the ring 3 and is free to move axially relative to the distal ring 3 somewhat in the manner of a pulley. The proximal ring 4 is fixed to the sleeve 2, in this case at the proximal inner end thereof. The sleeve 2 terminates in a handle or gripping portion which in this case is reinforced by a gripping ring 15.

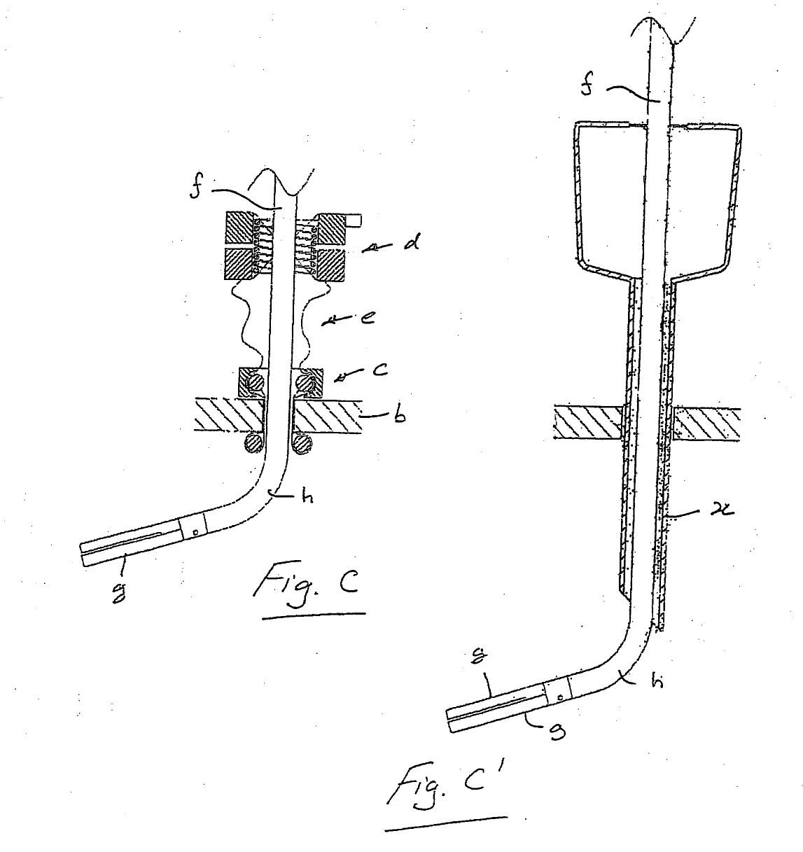

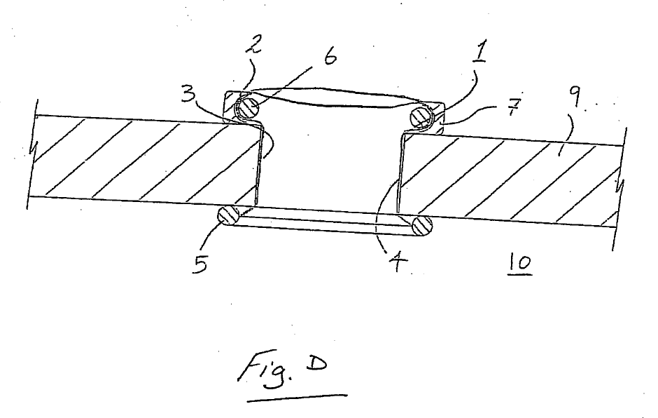

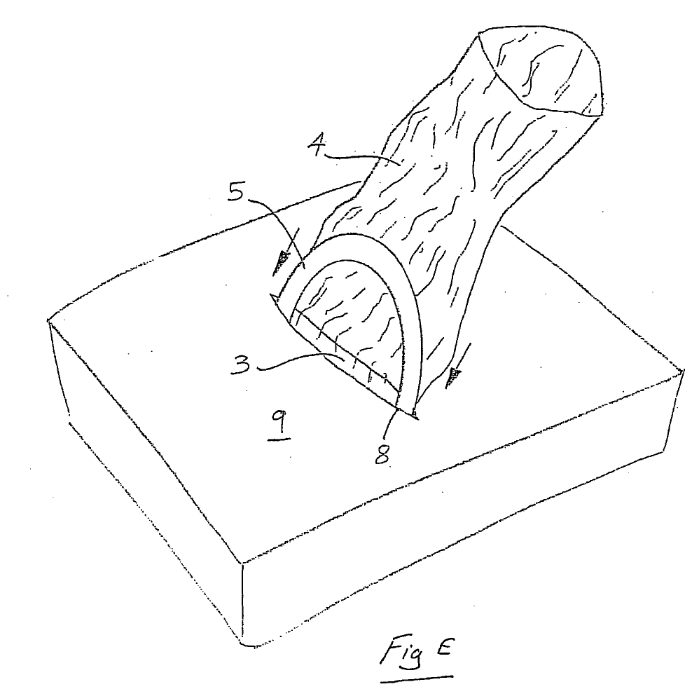

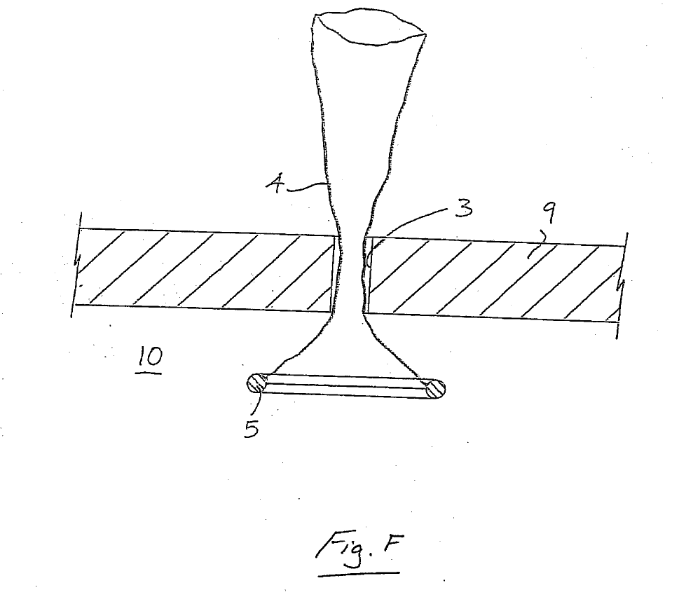

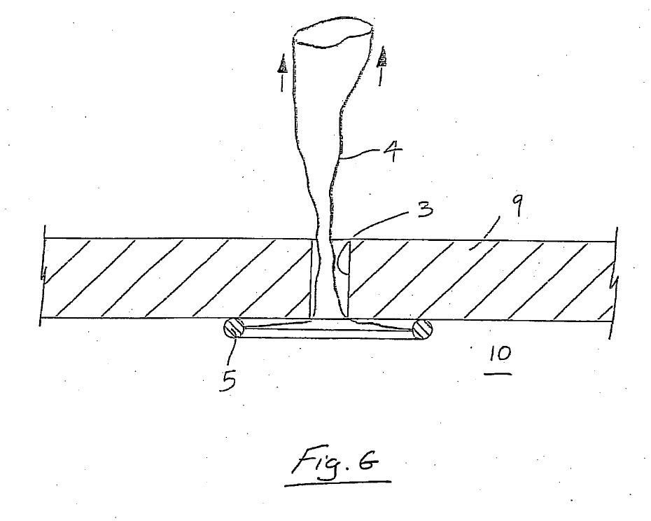

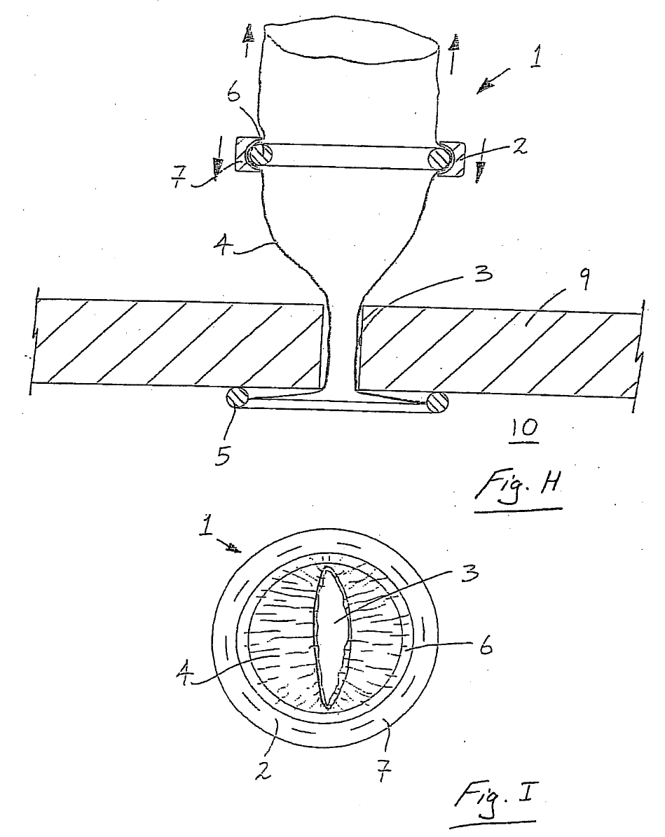

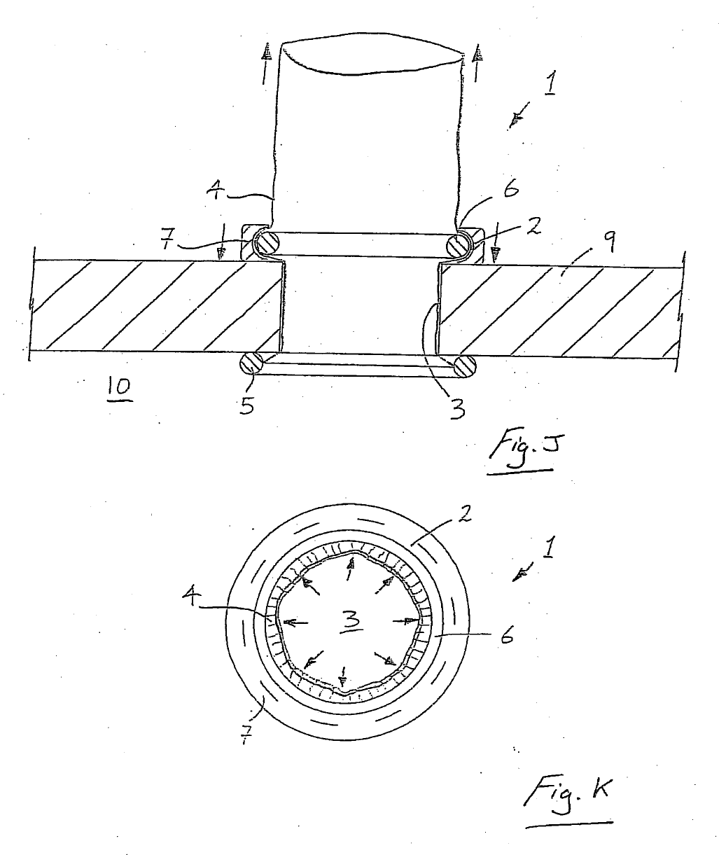

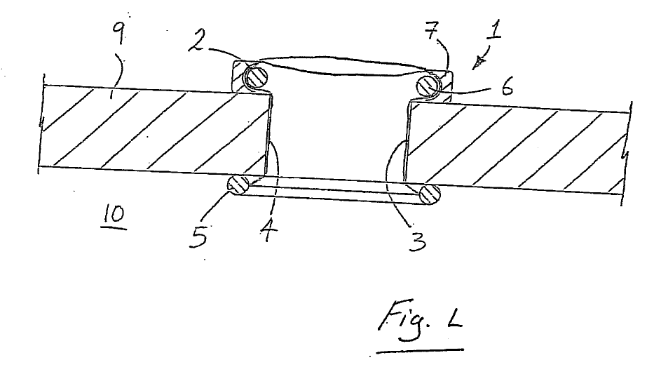

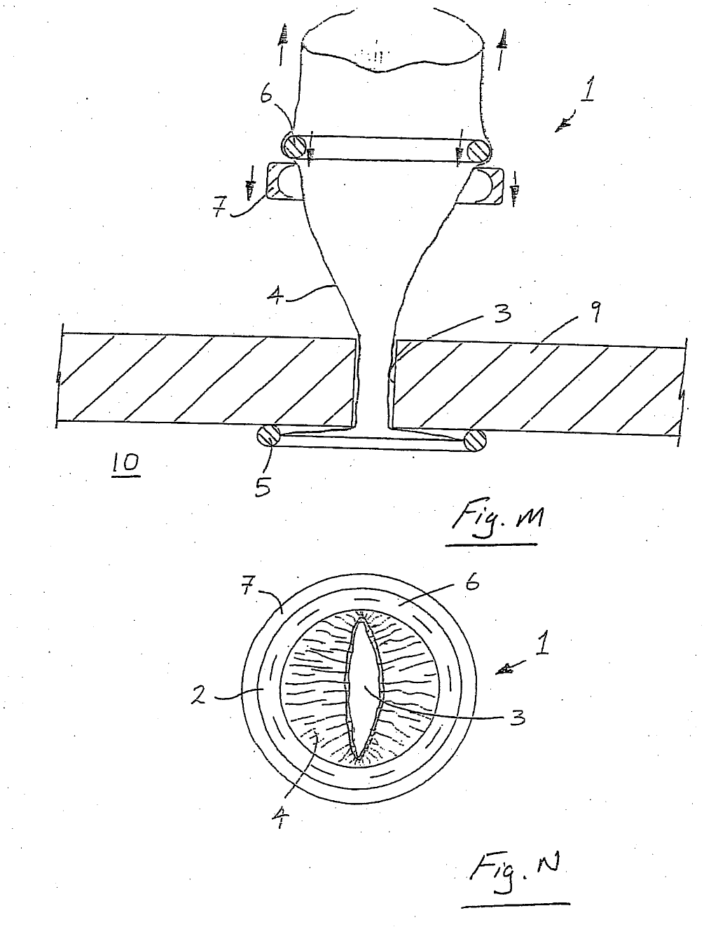

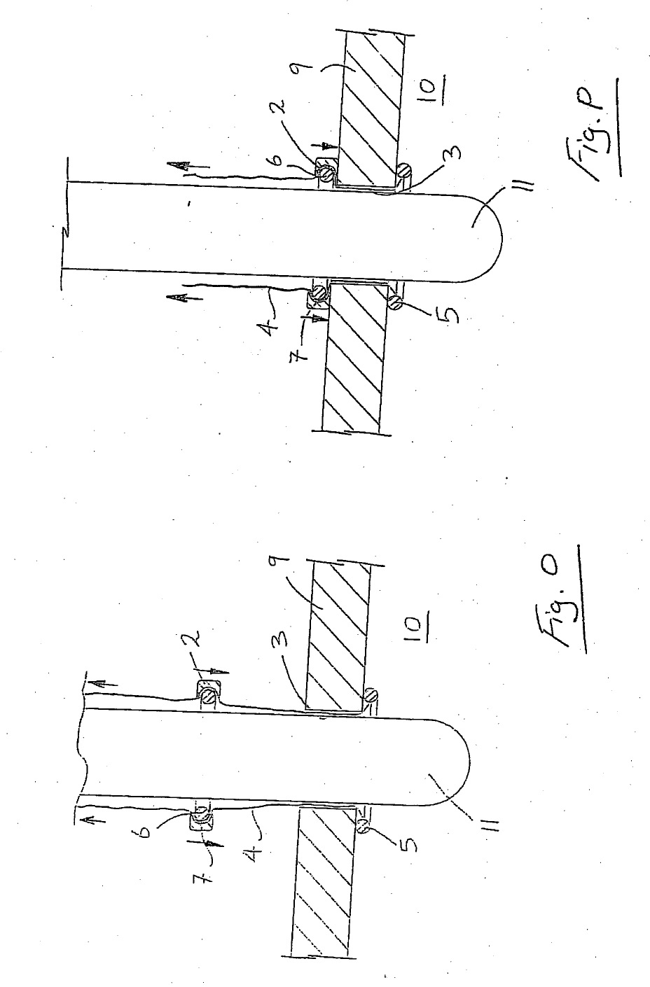

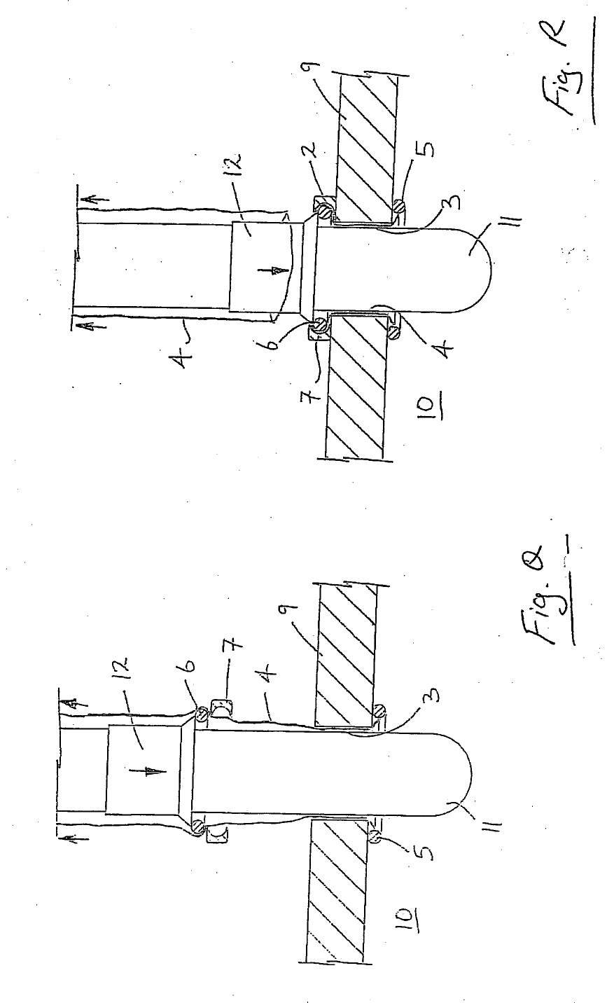

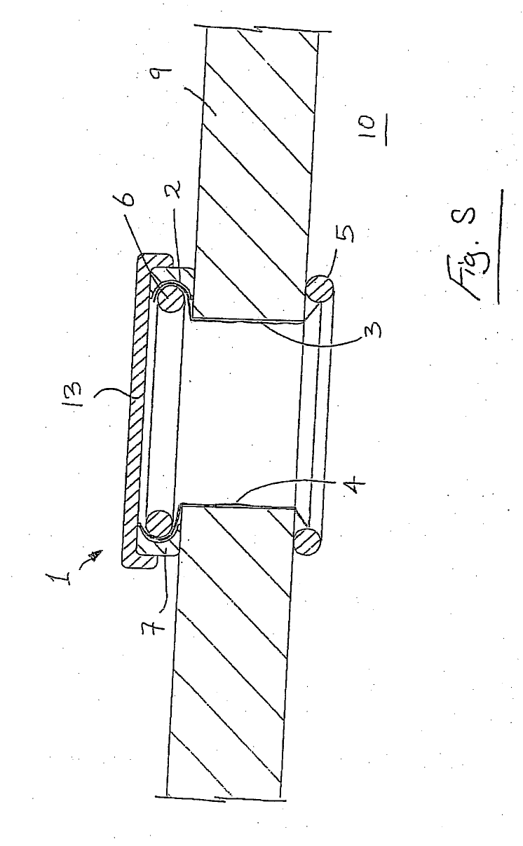

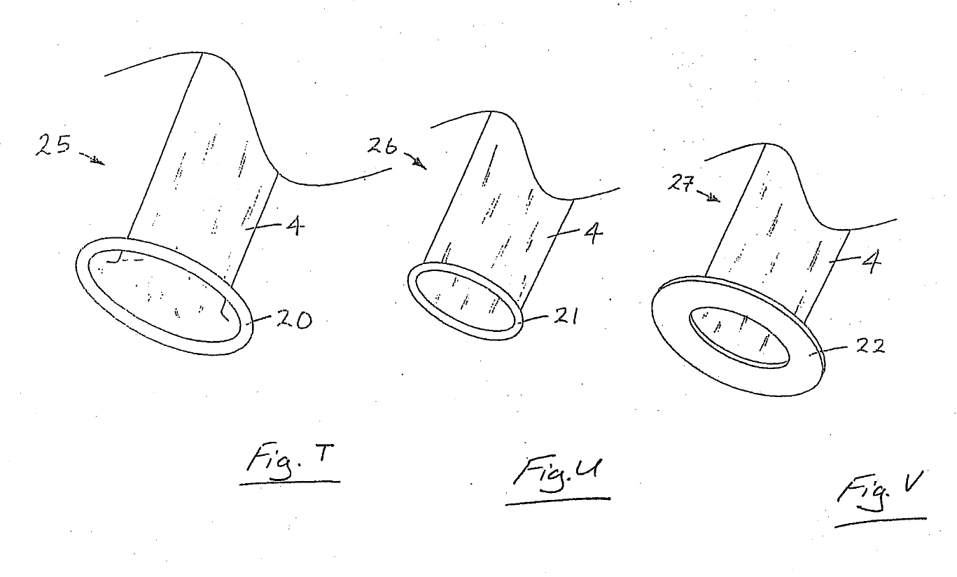

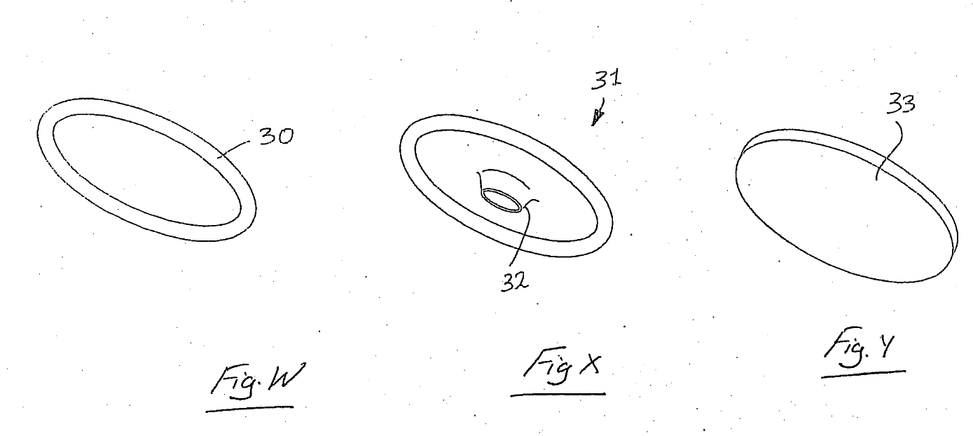

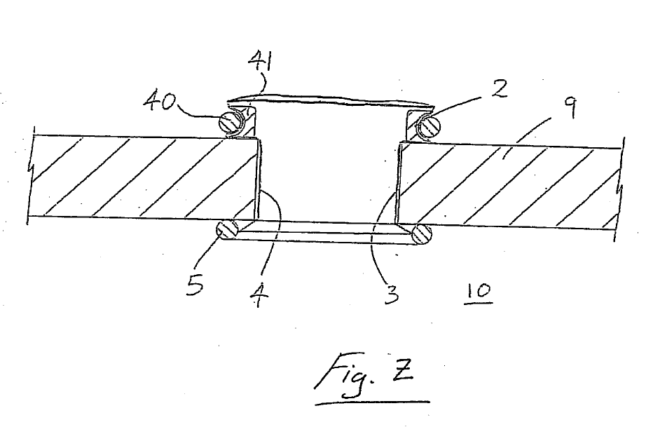

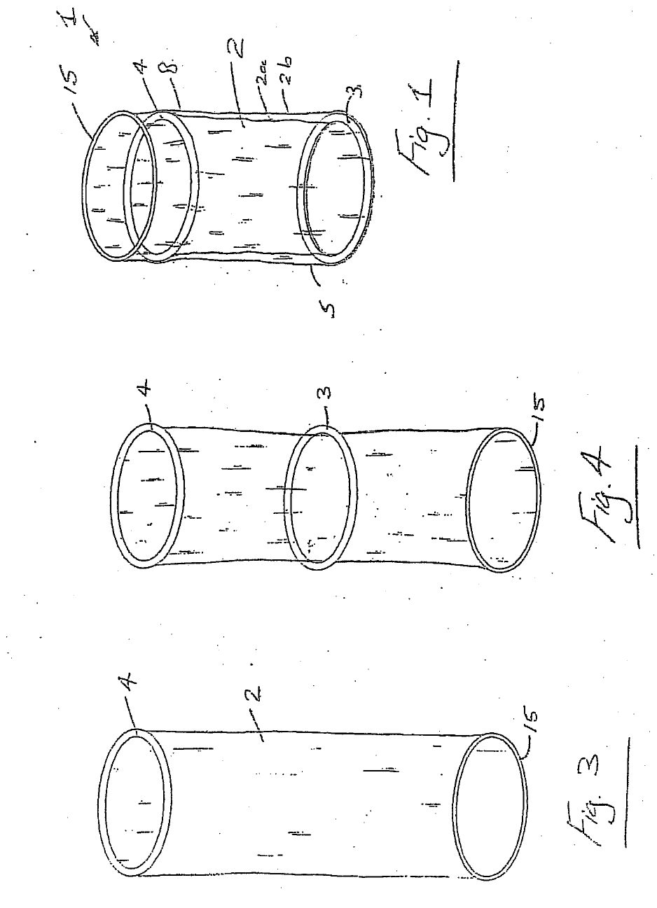

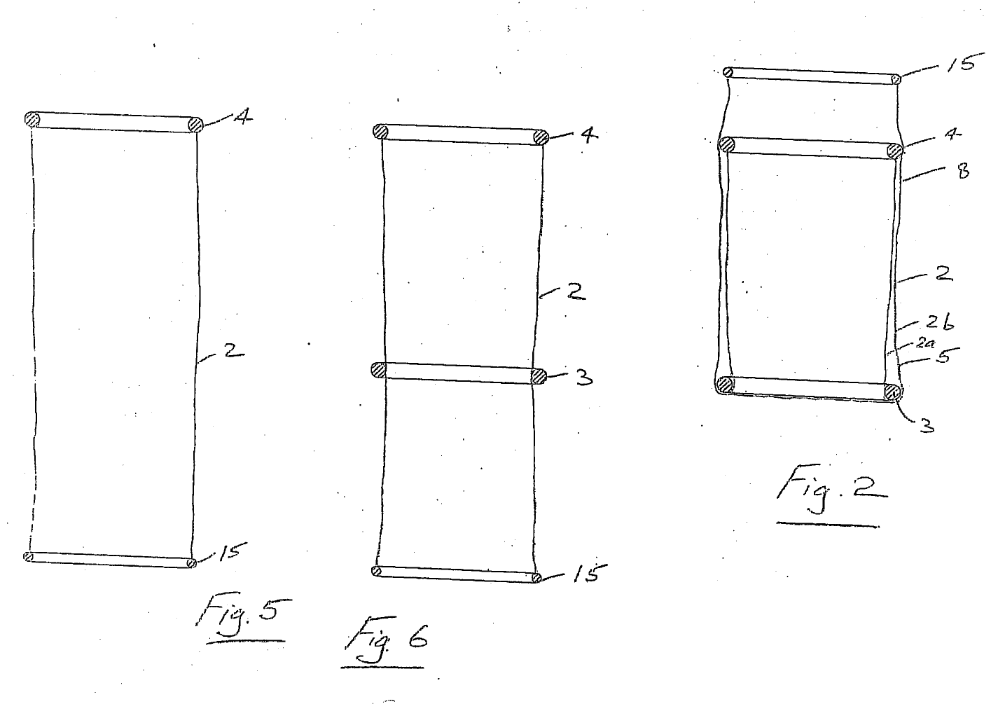

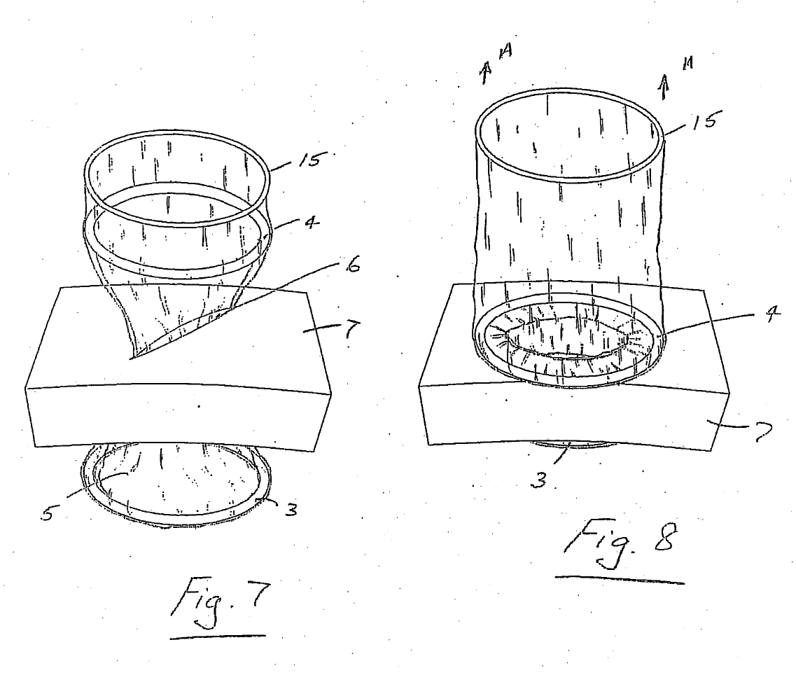

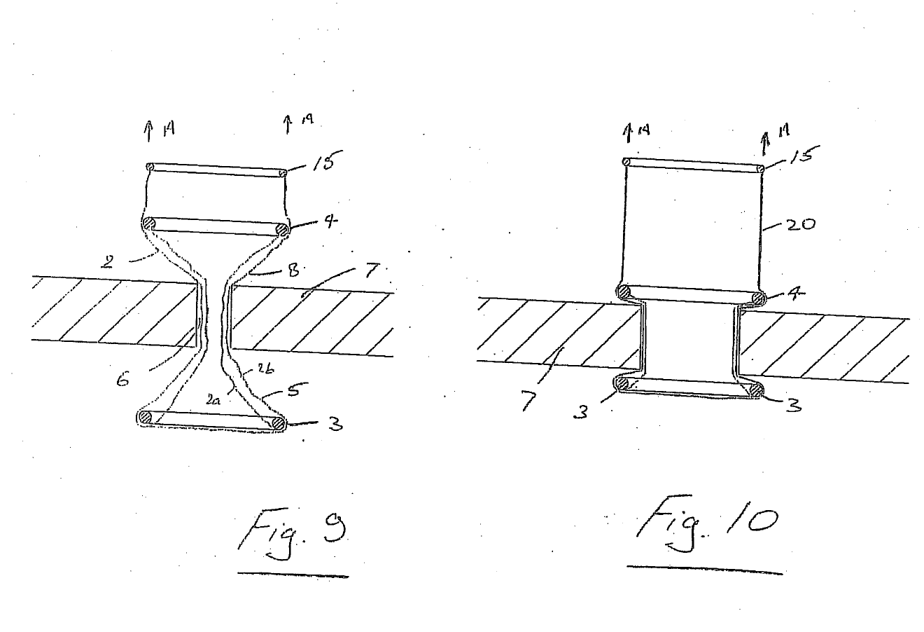

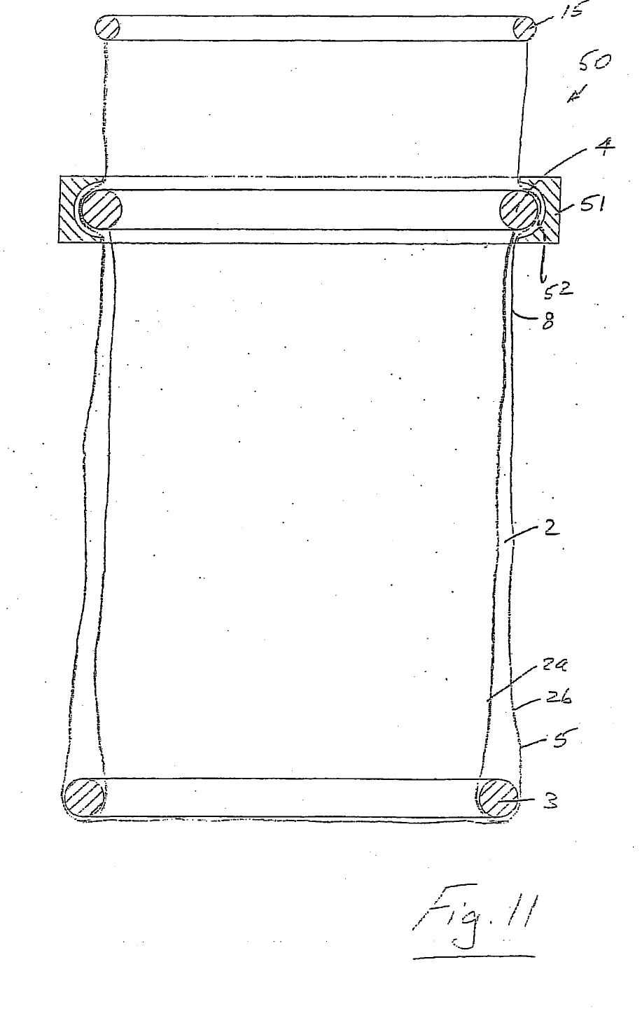

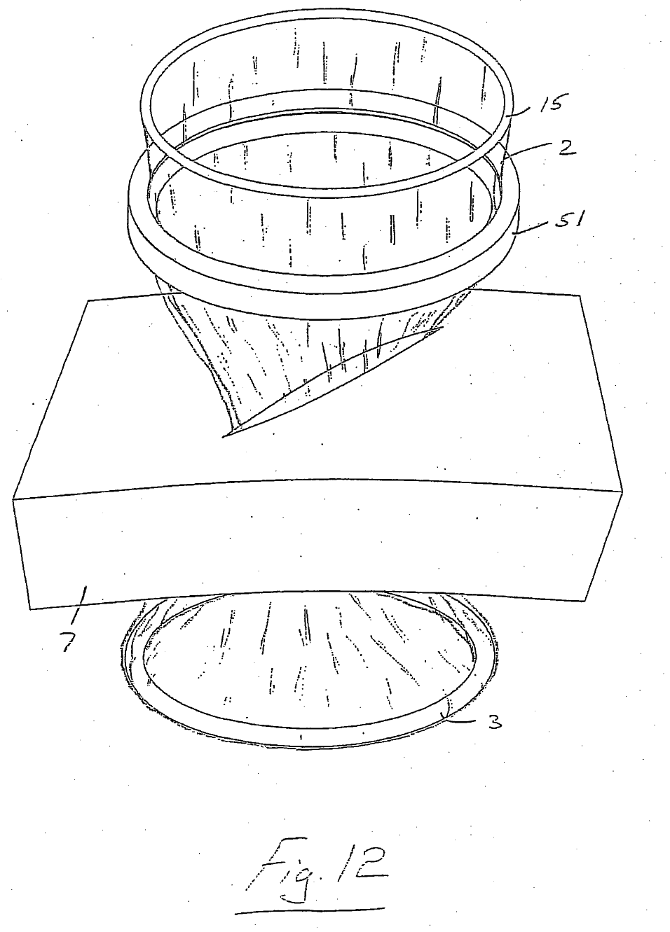

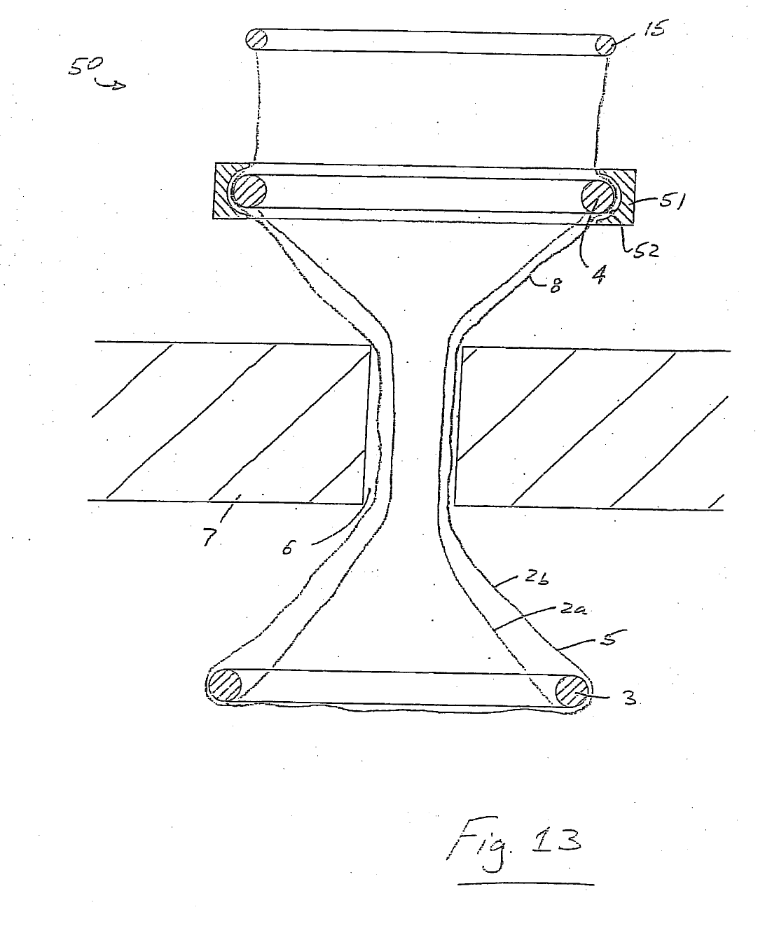

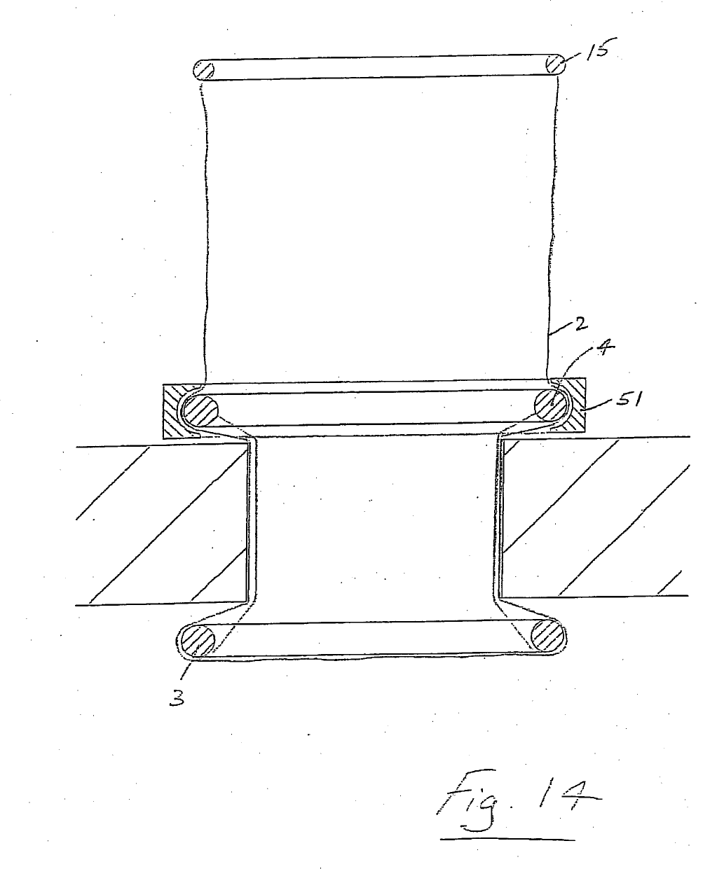

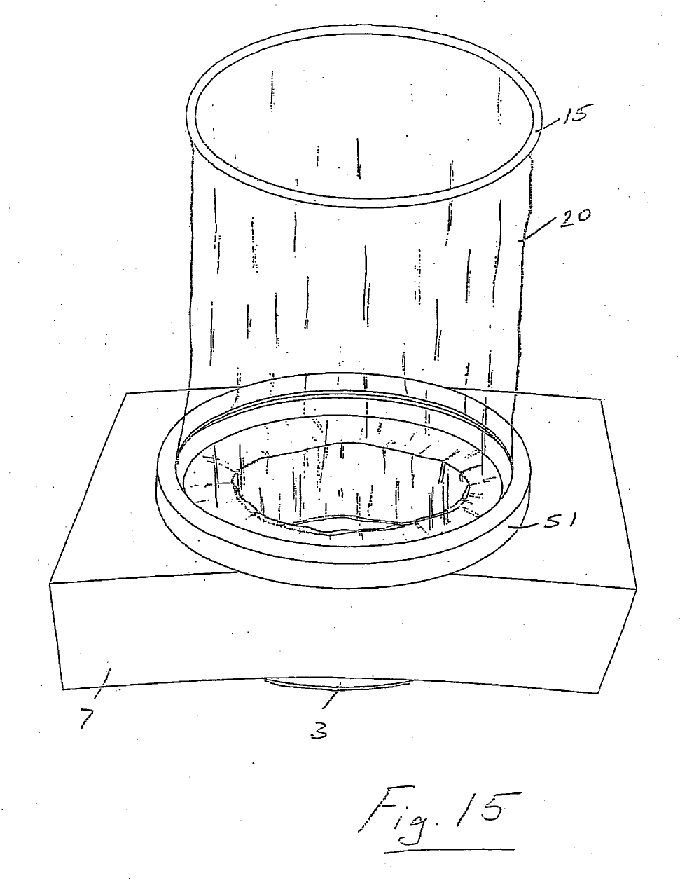

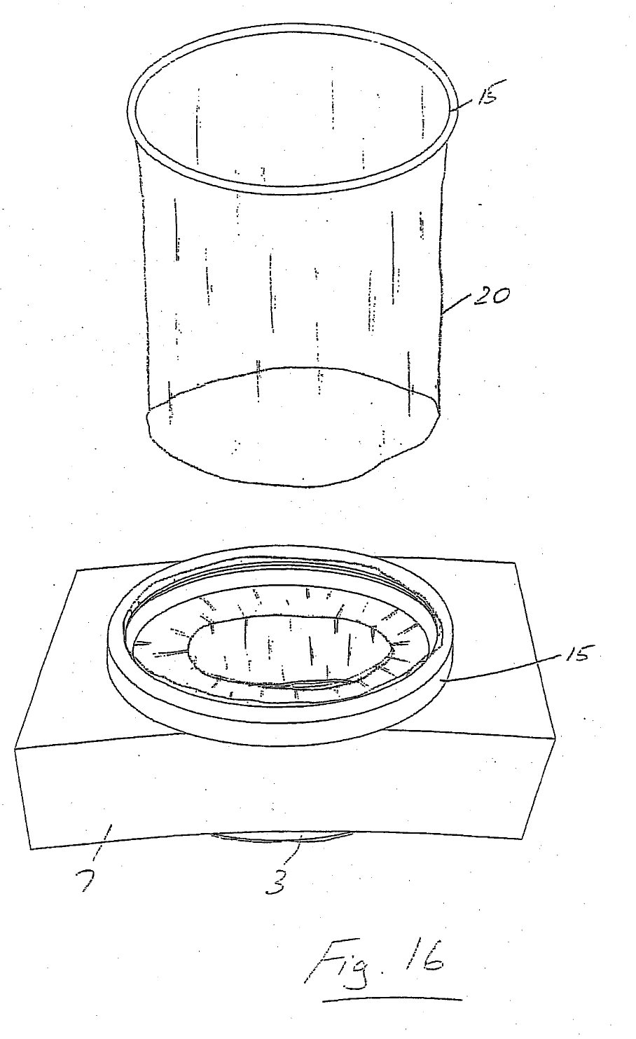

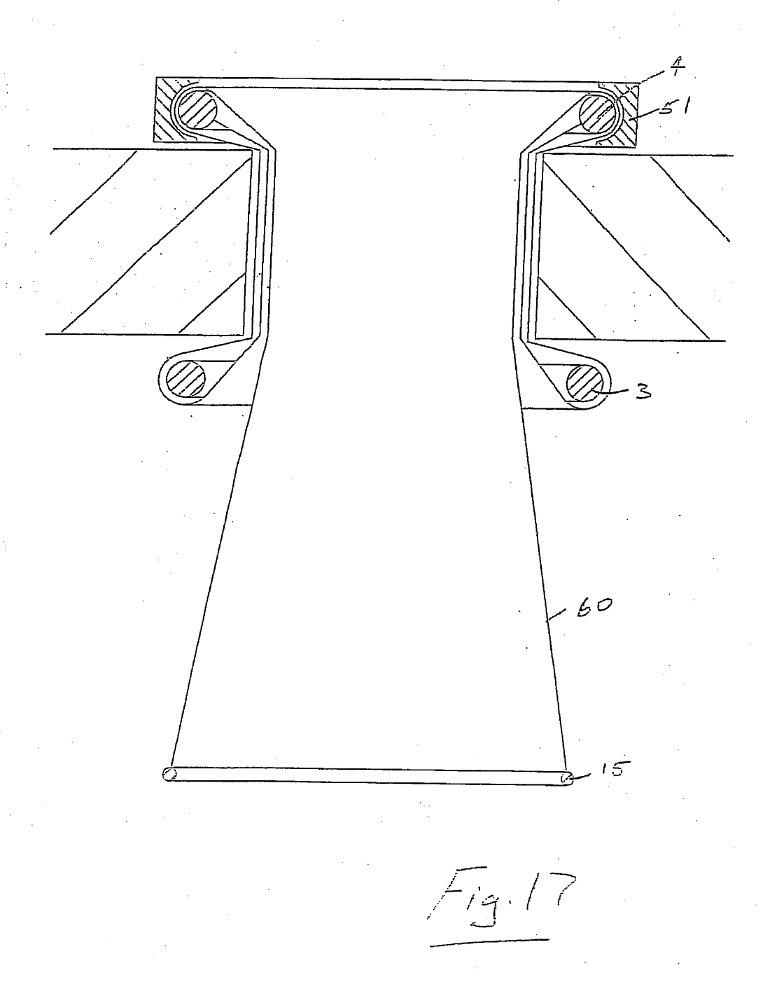

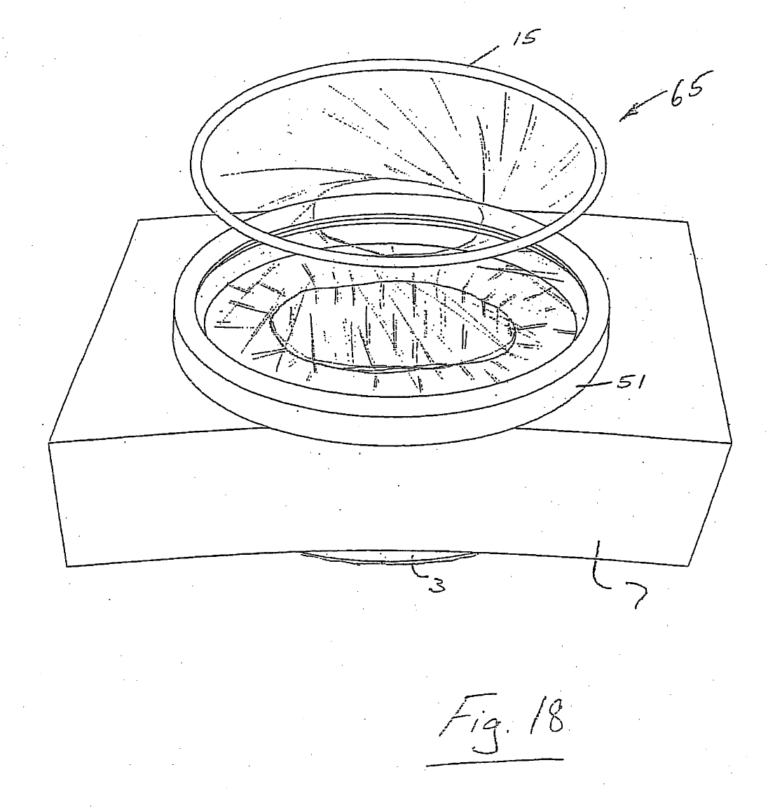

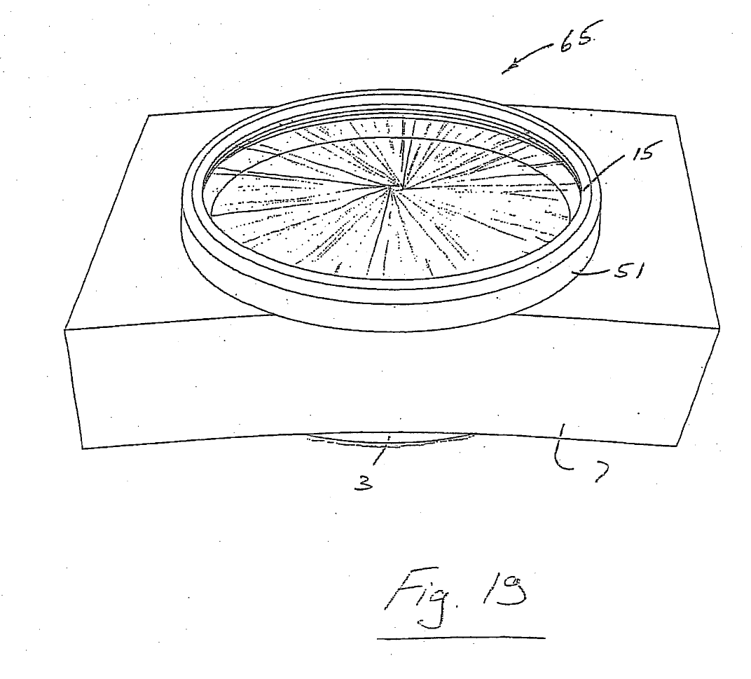

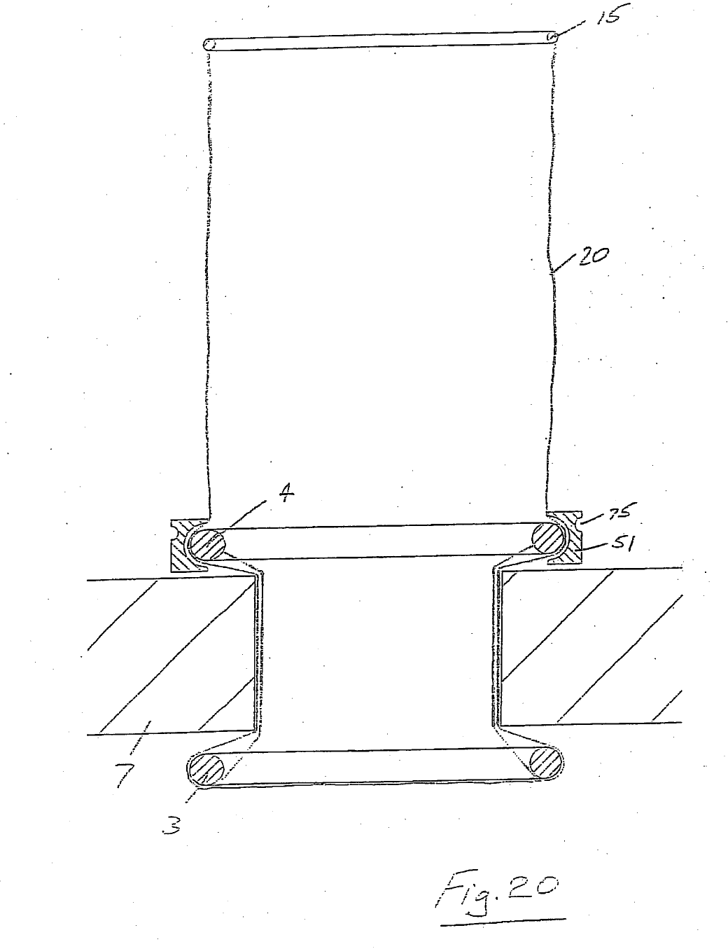

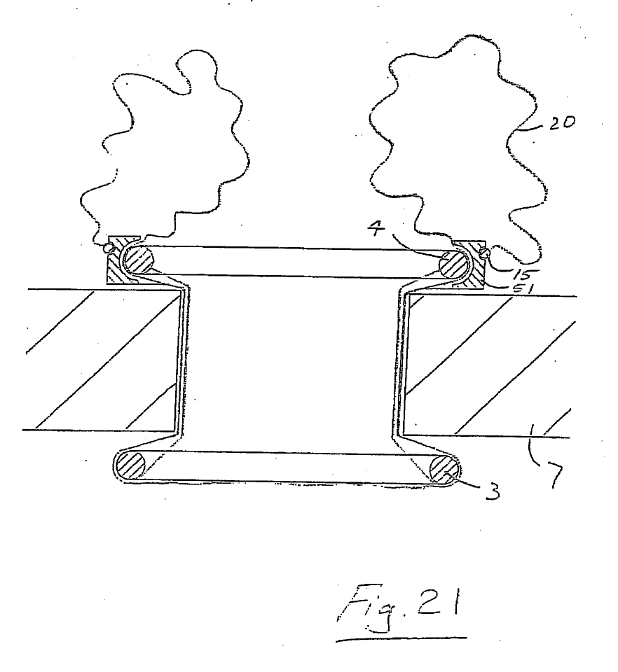

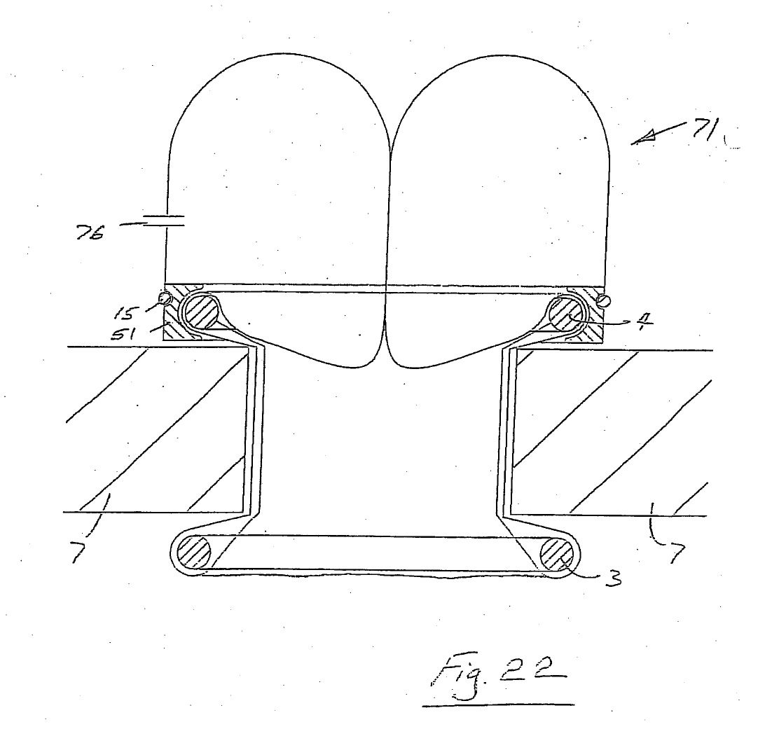

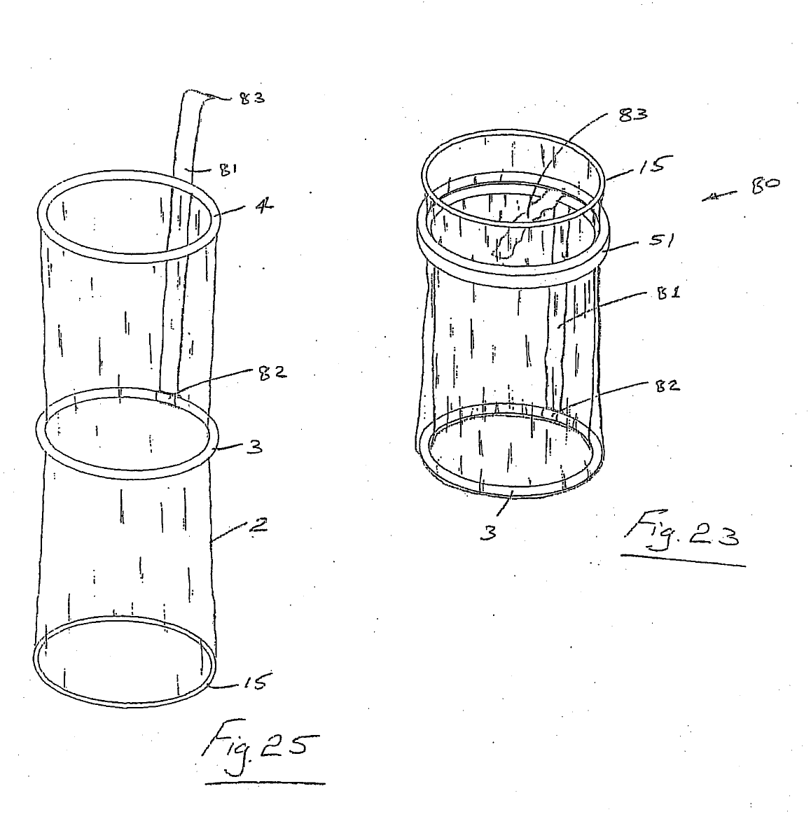

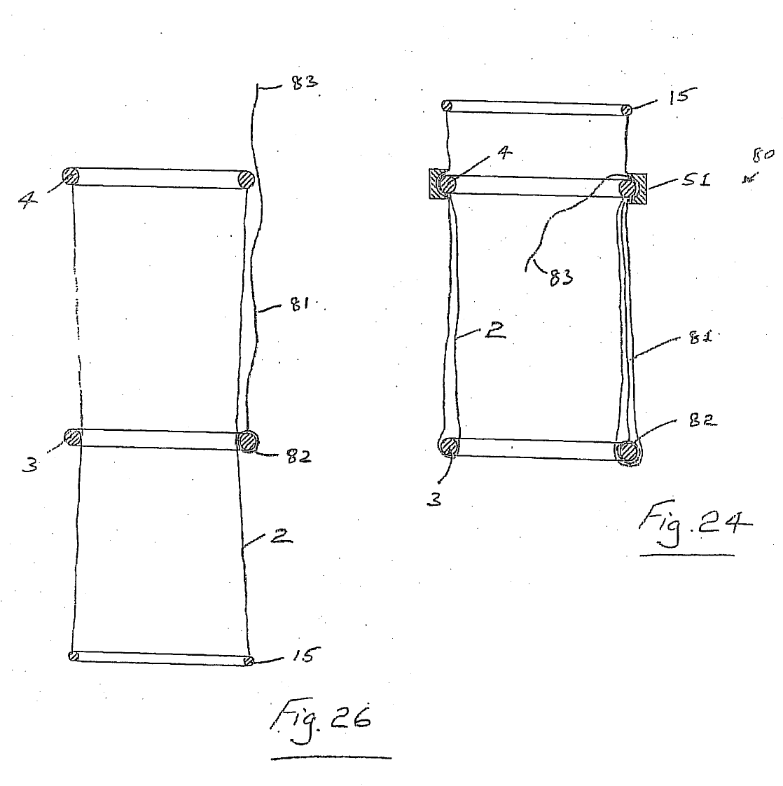

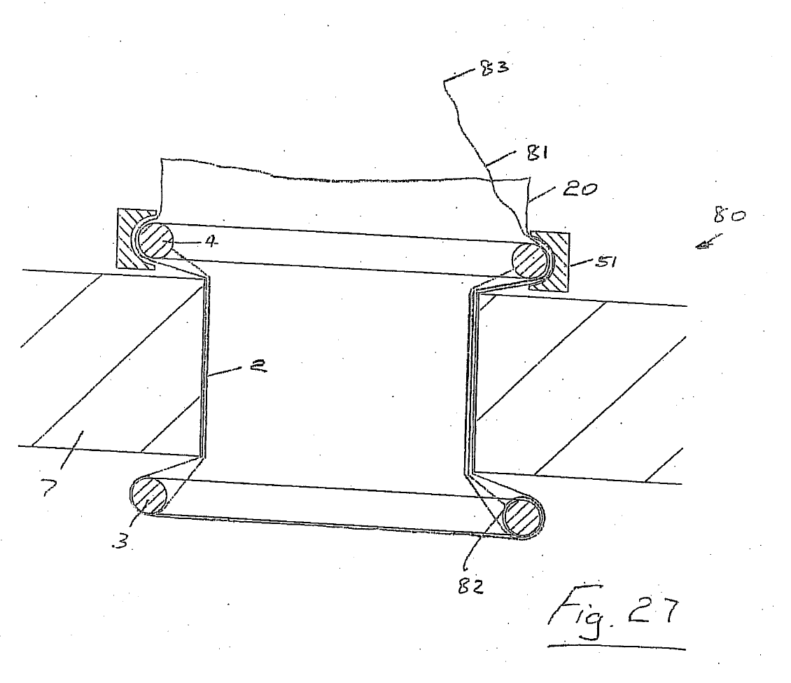

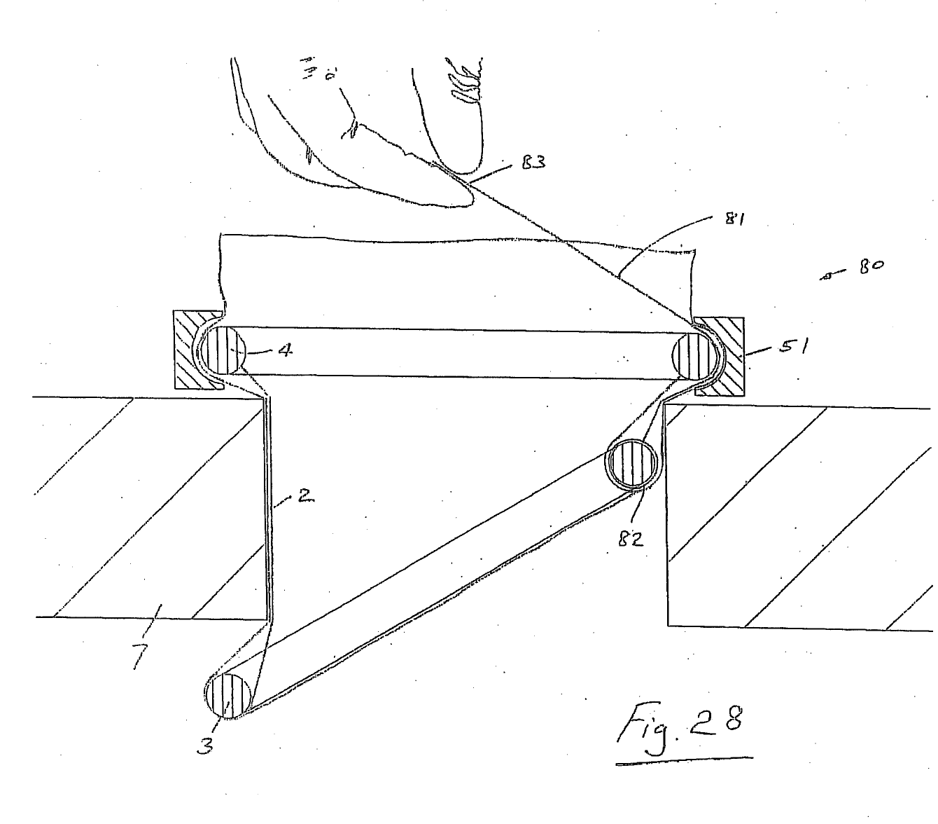

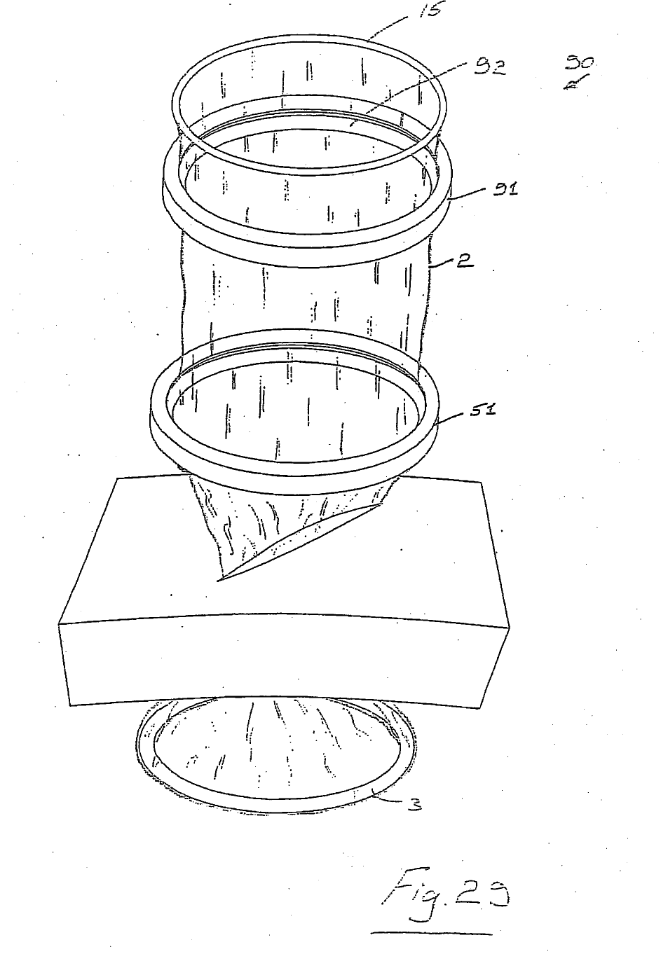

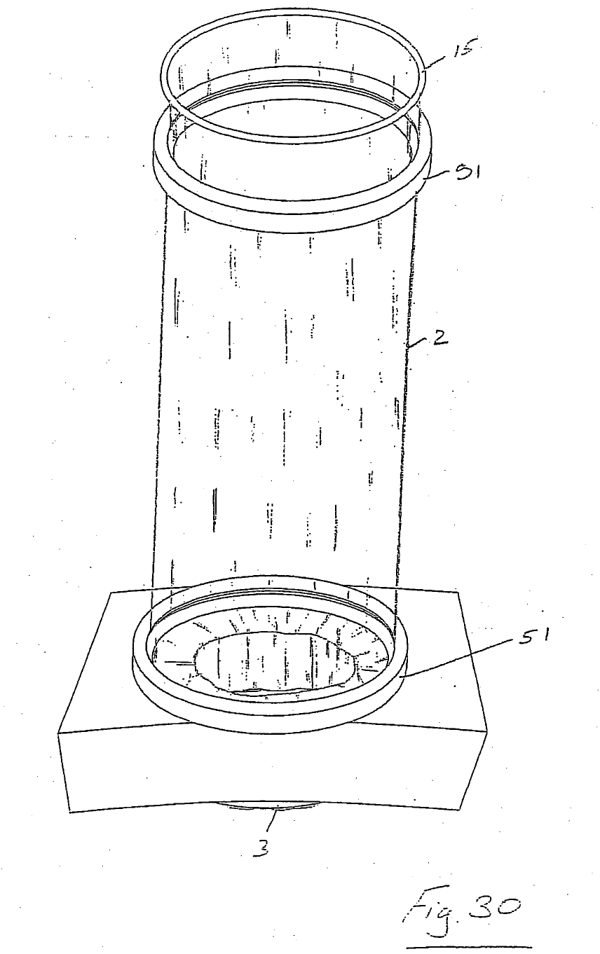

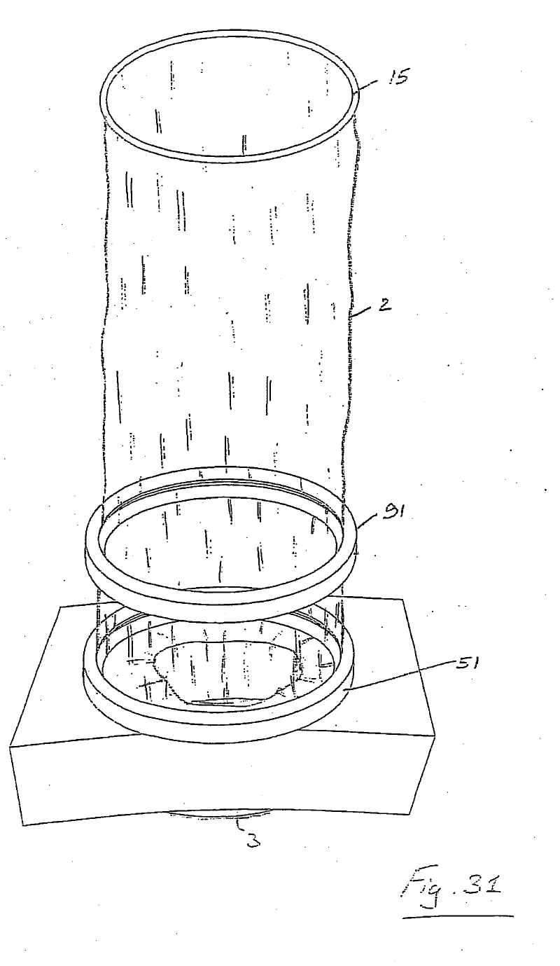

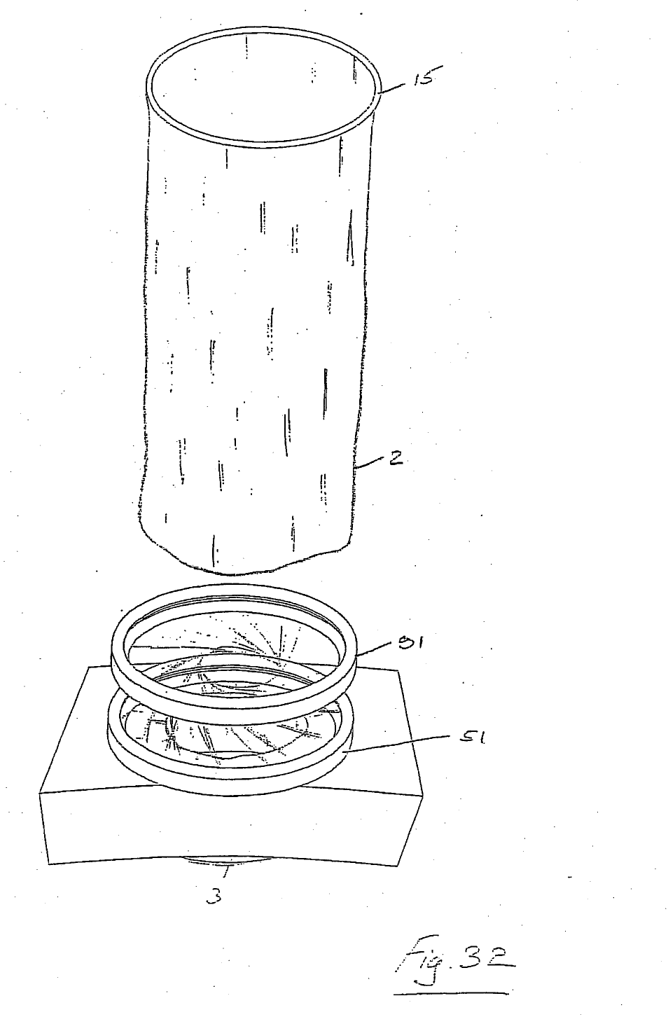

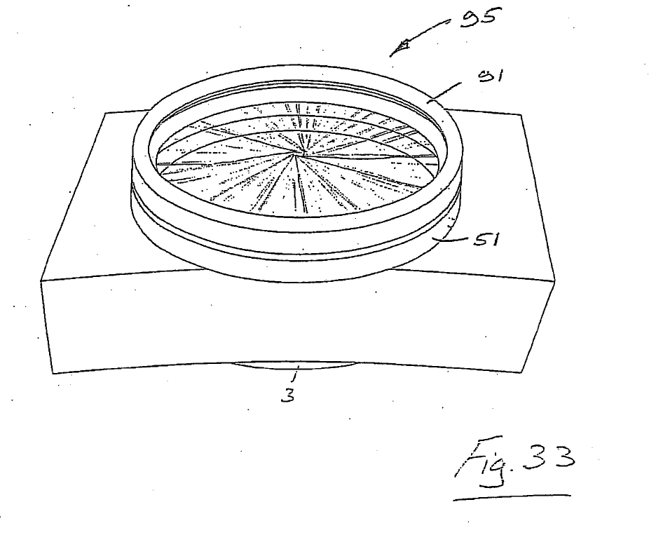

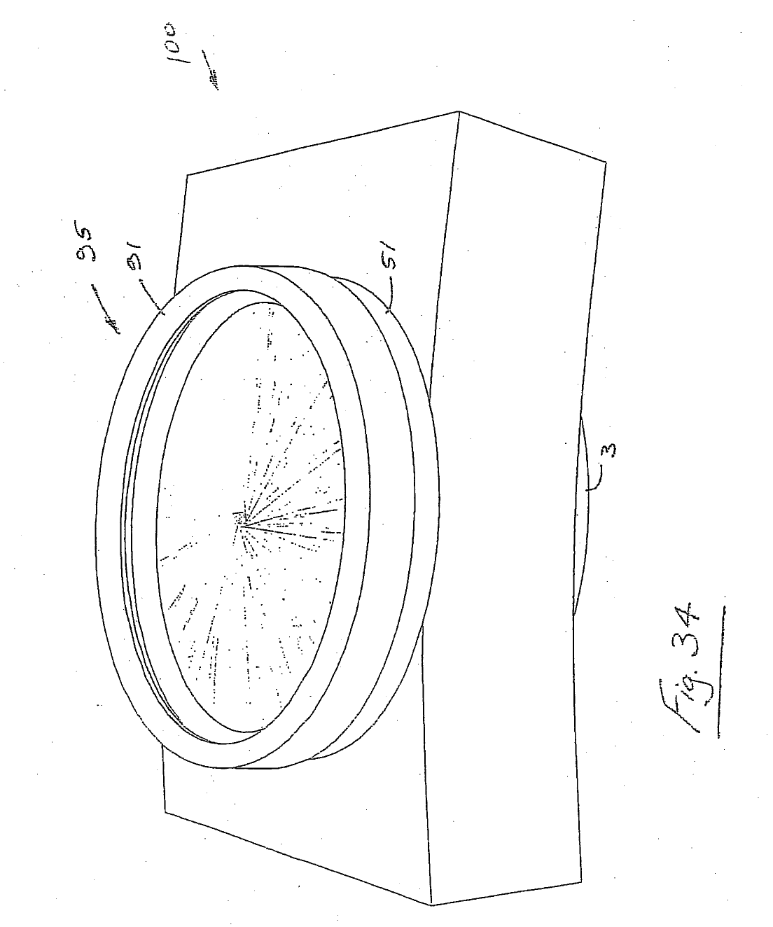

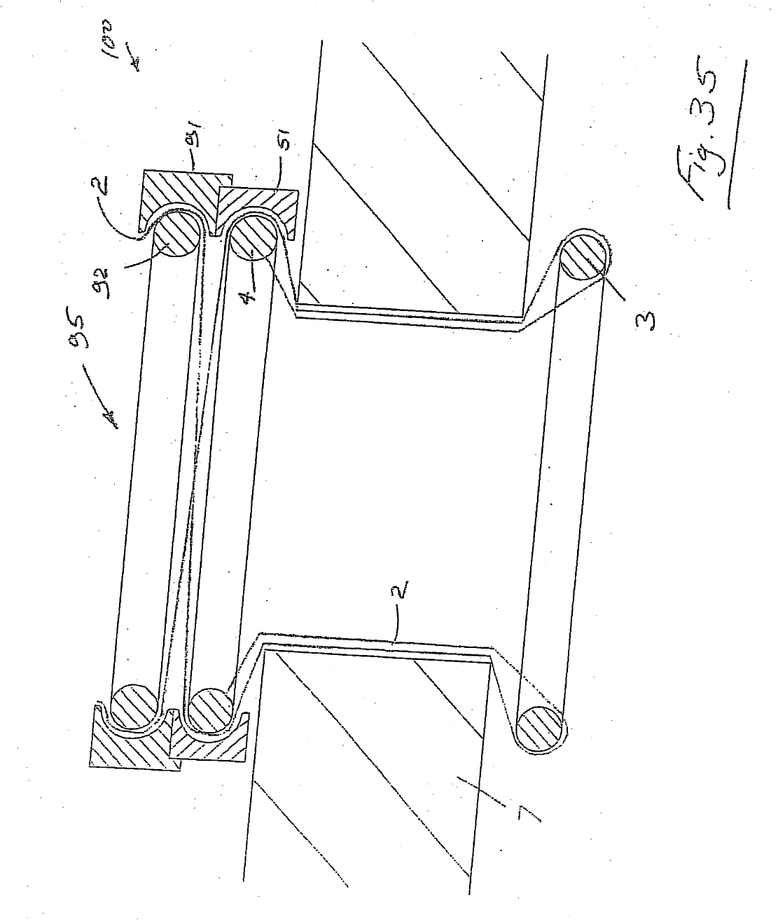

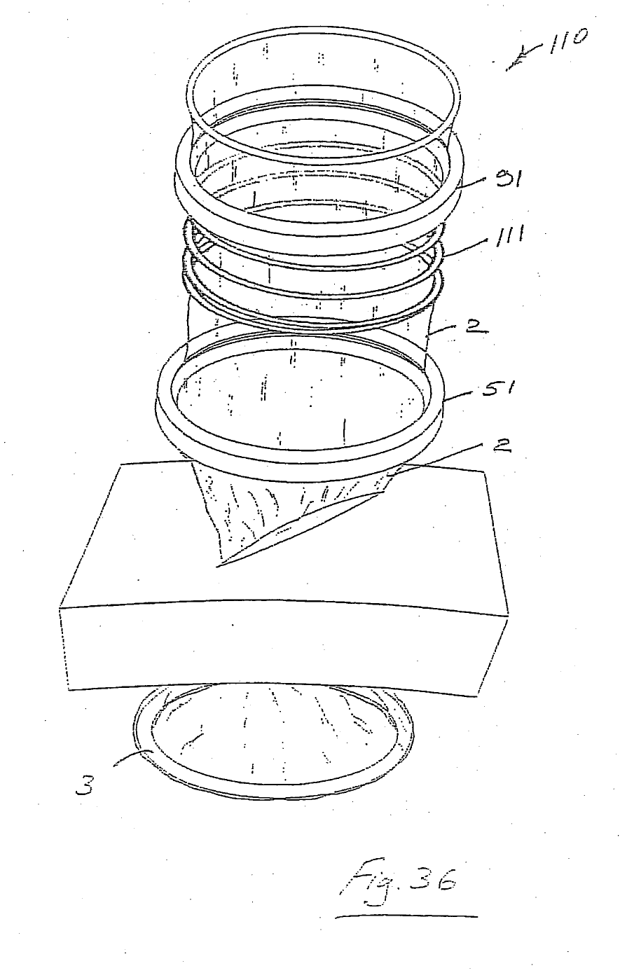

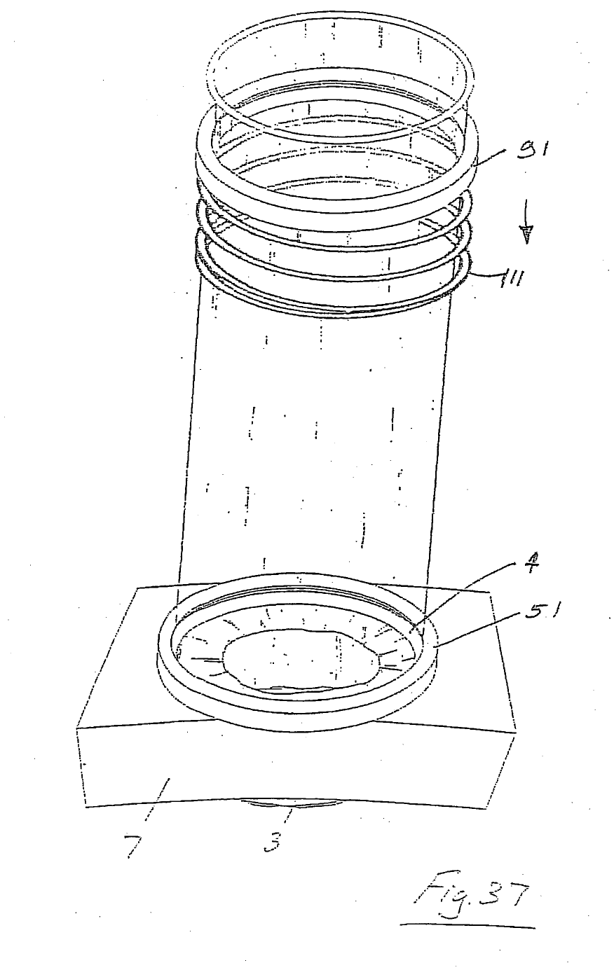

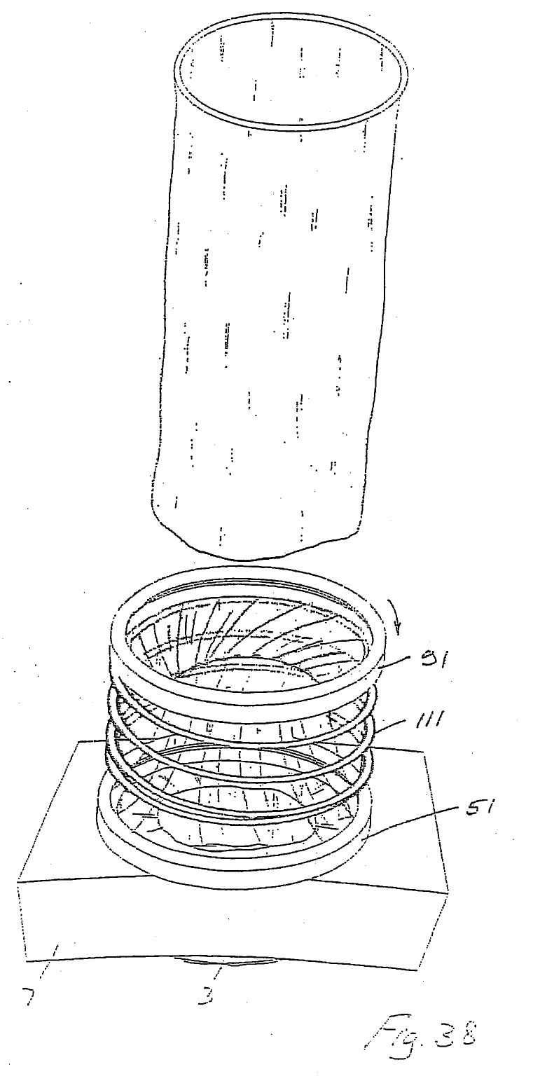

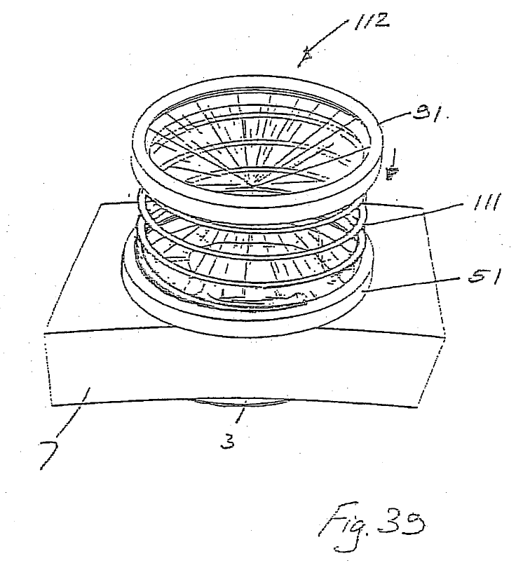

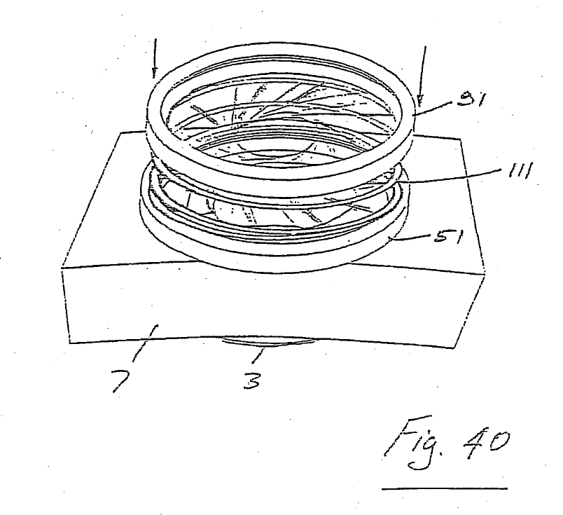

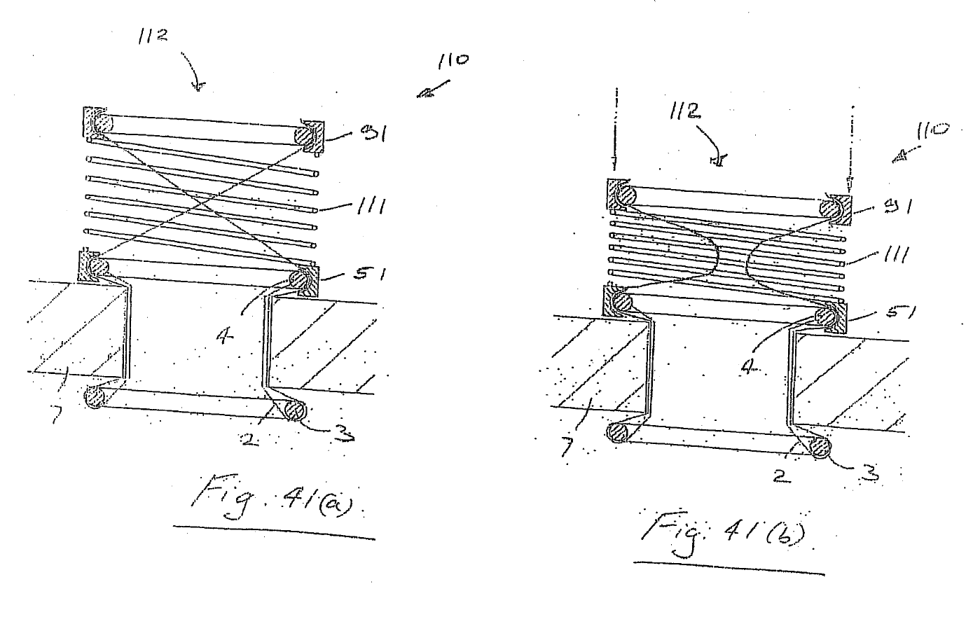

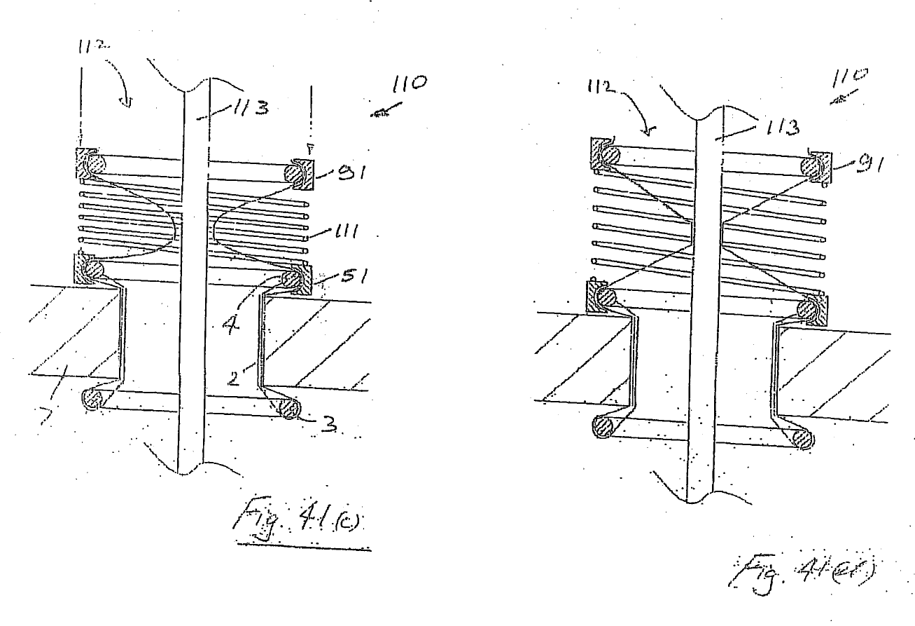

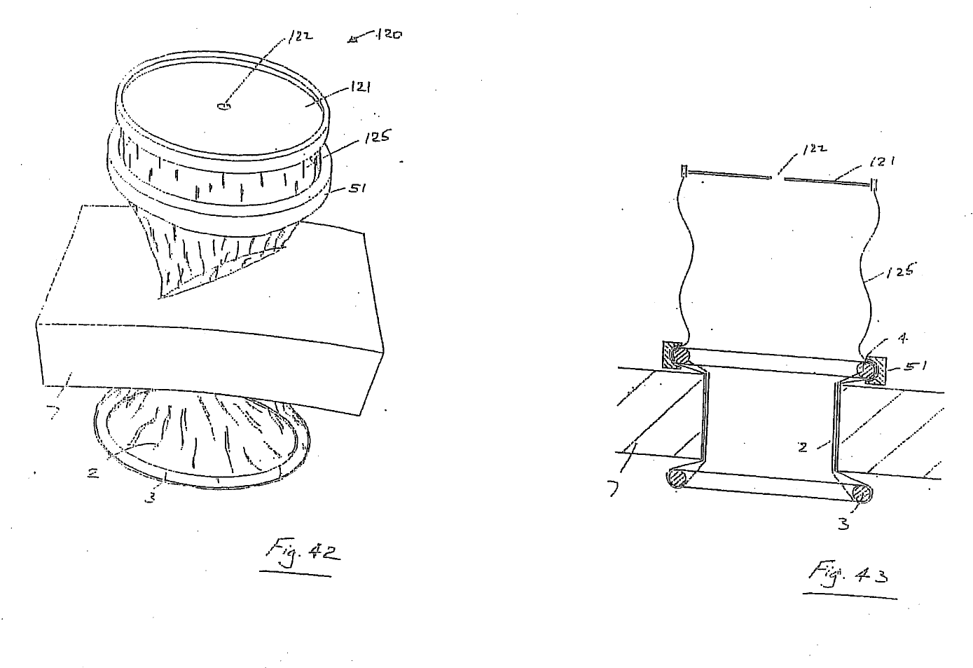

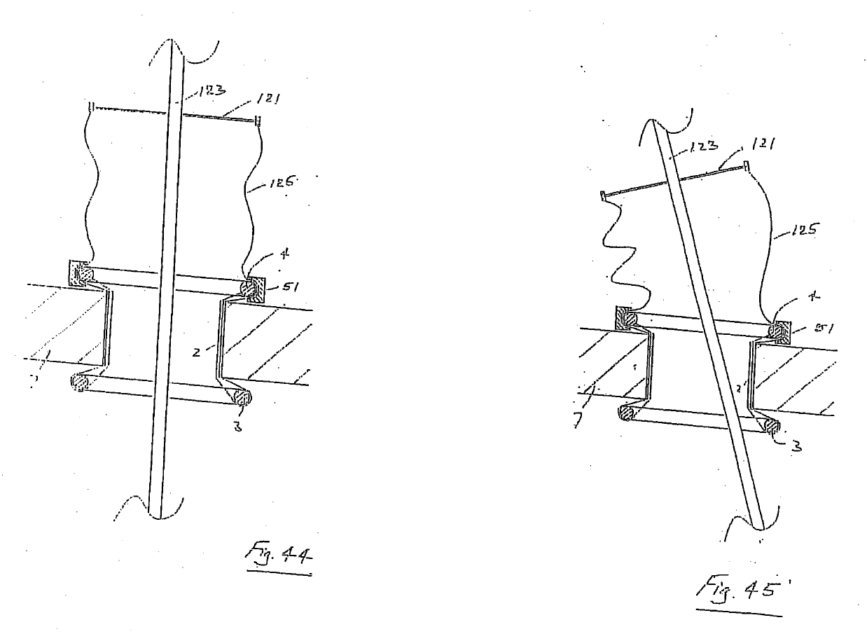

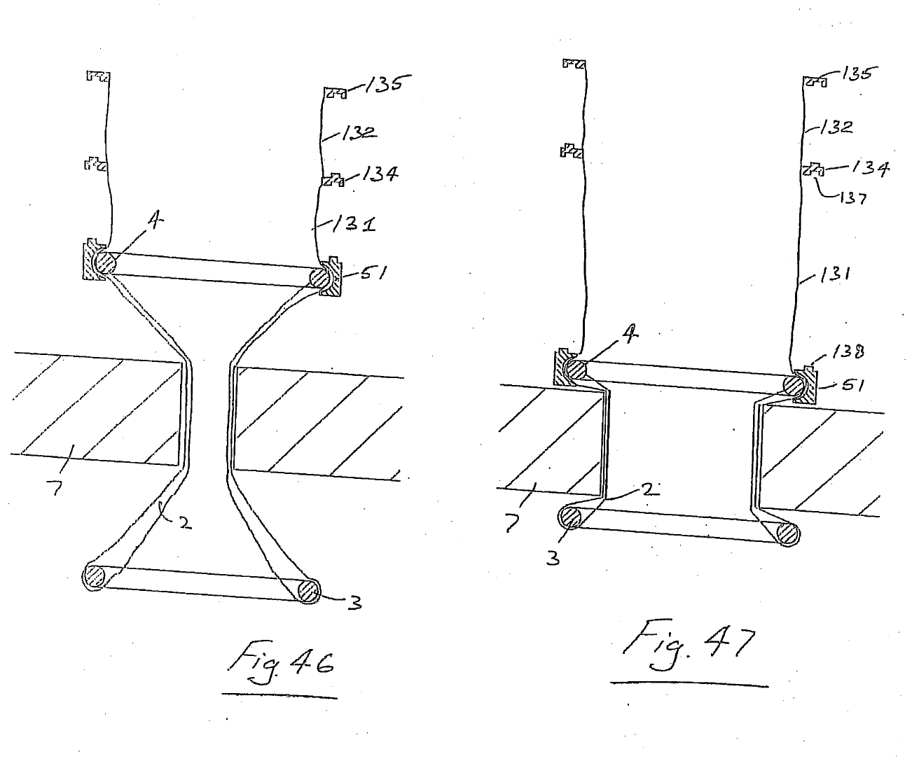

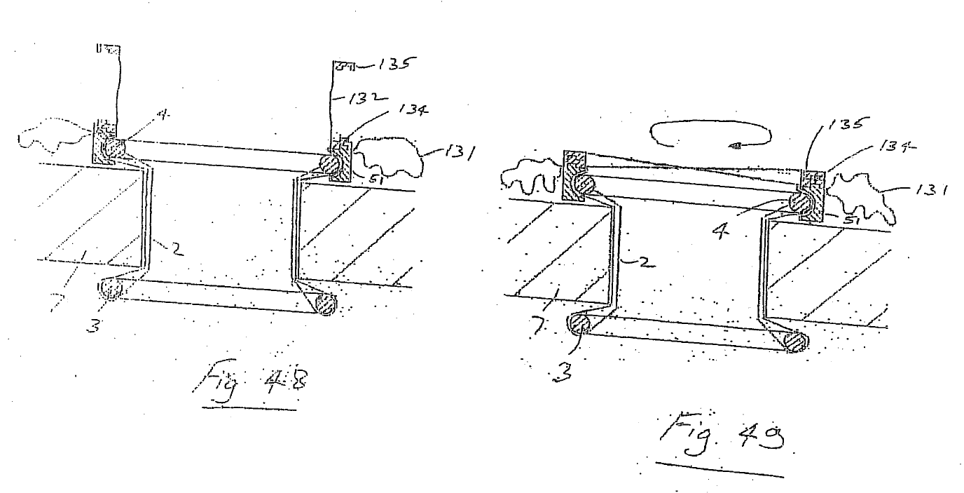

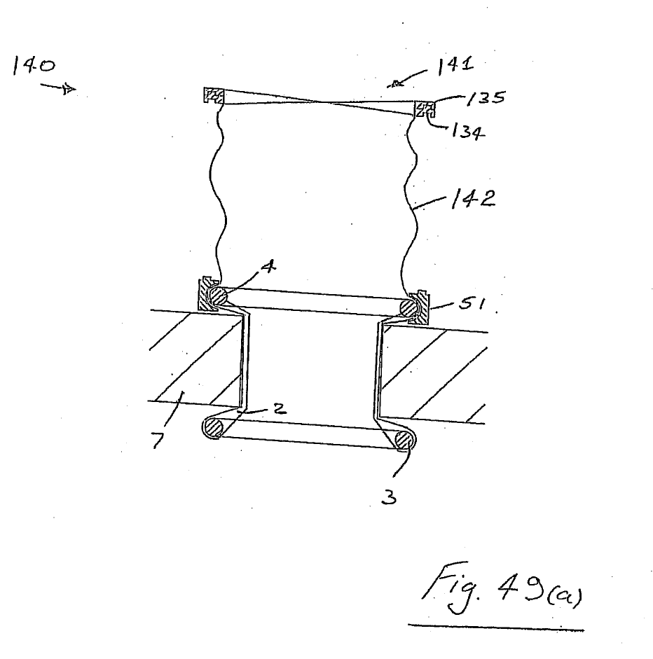

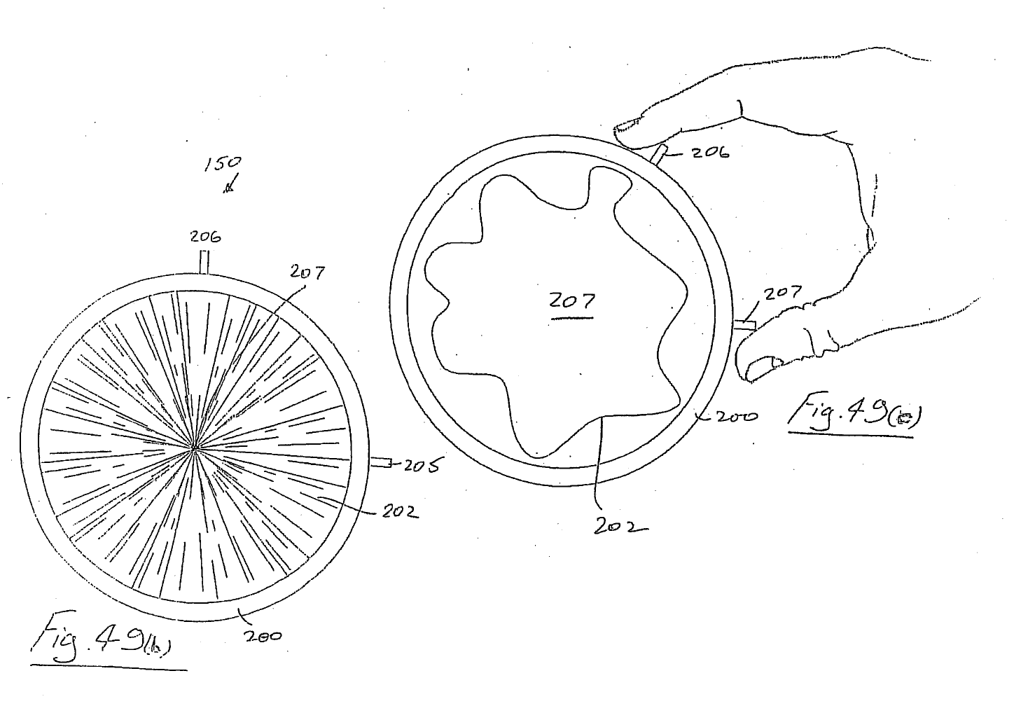

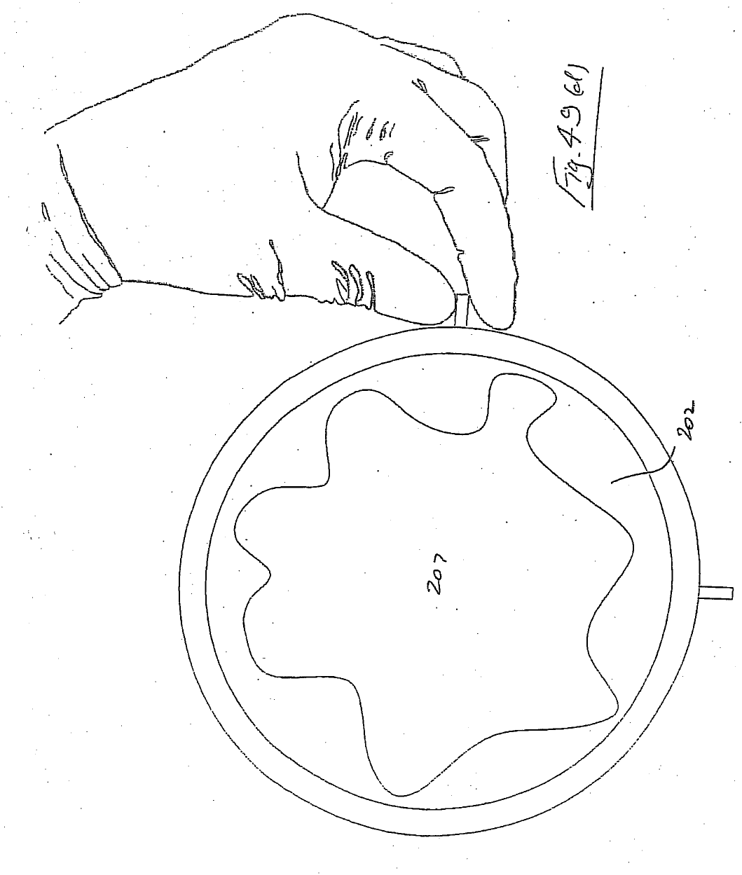

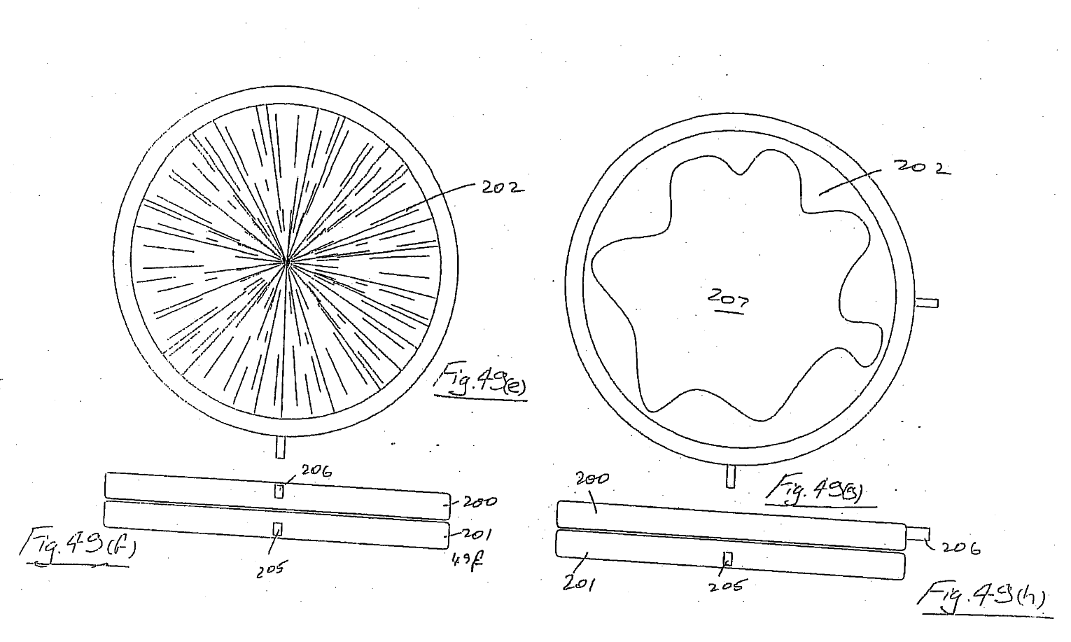

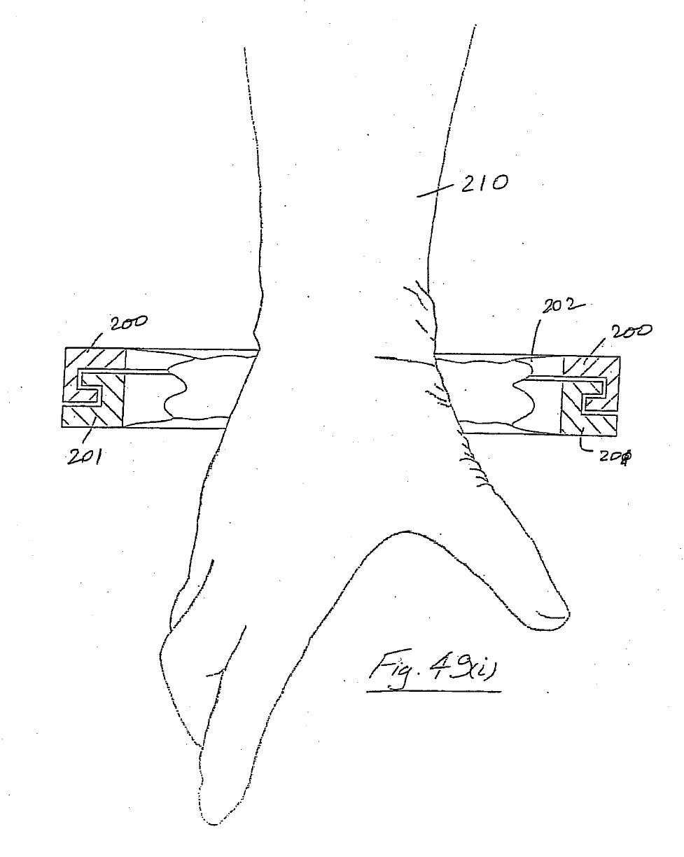

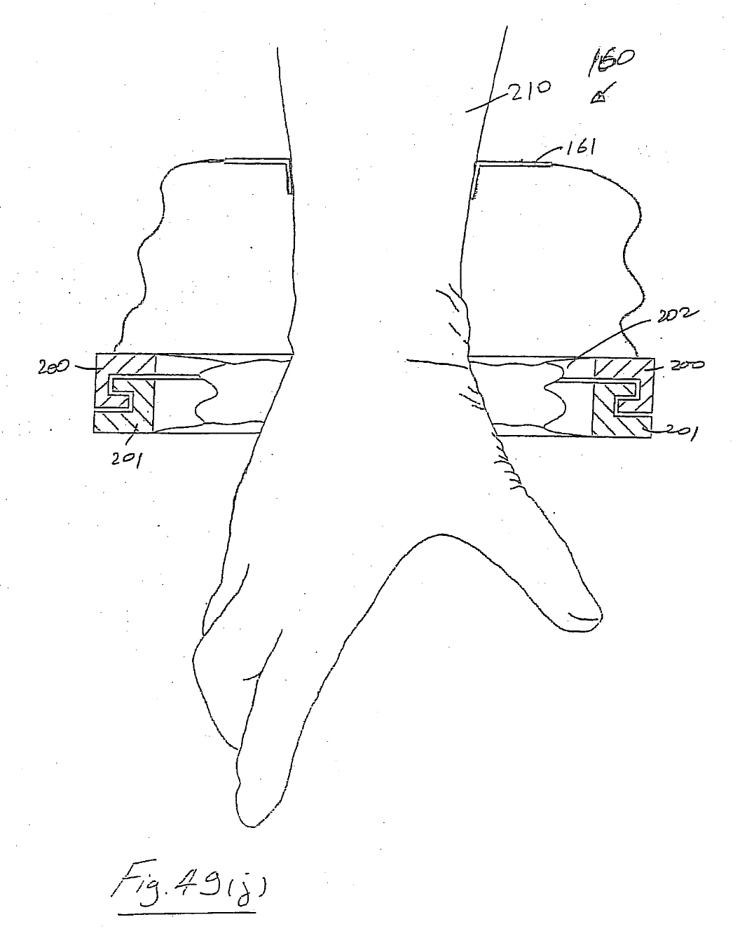

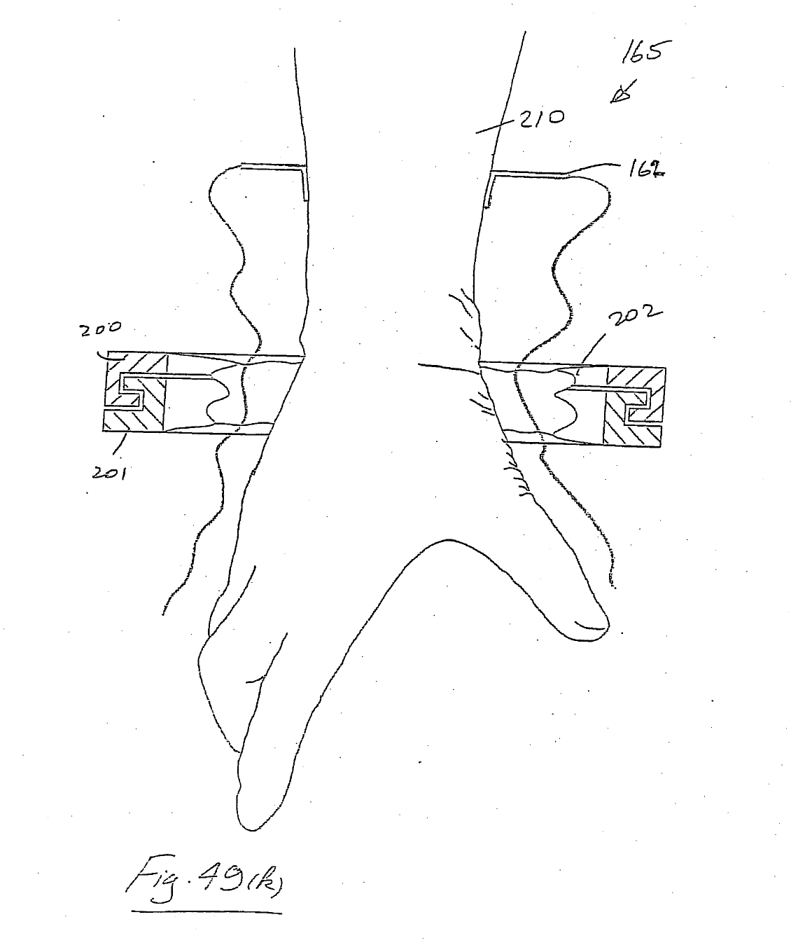

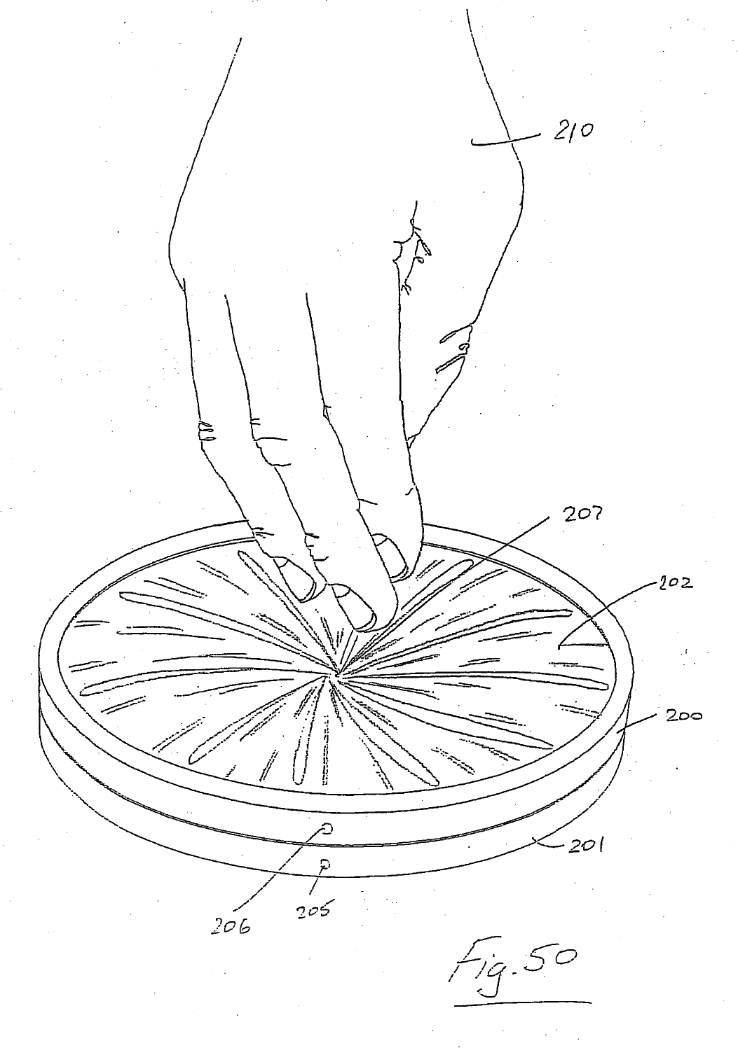

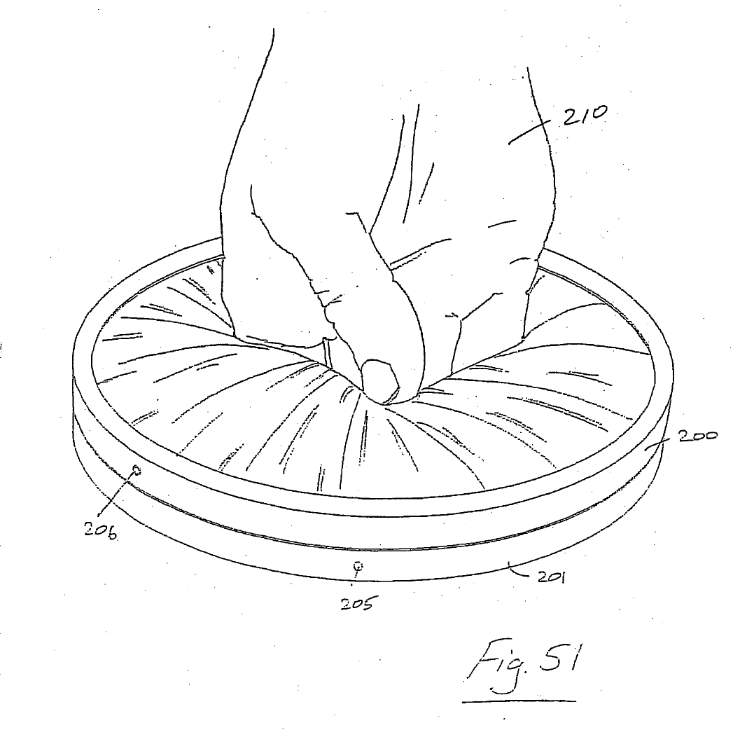

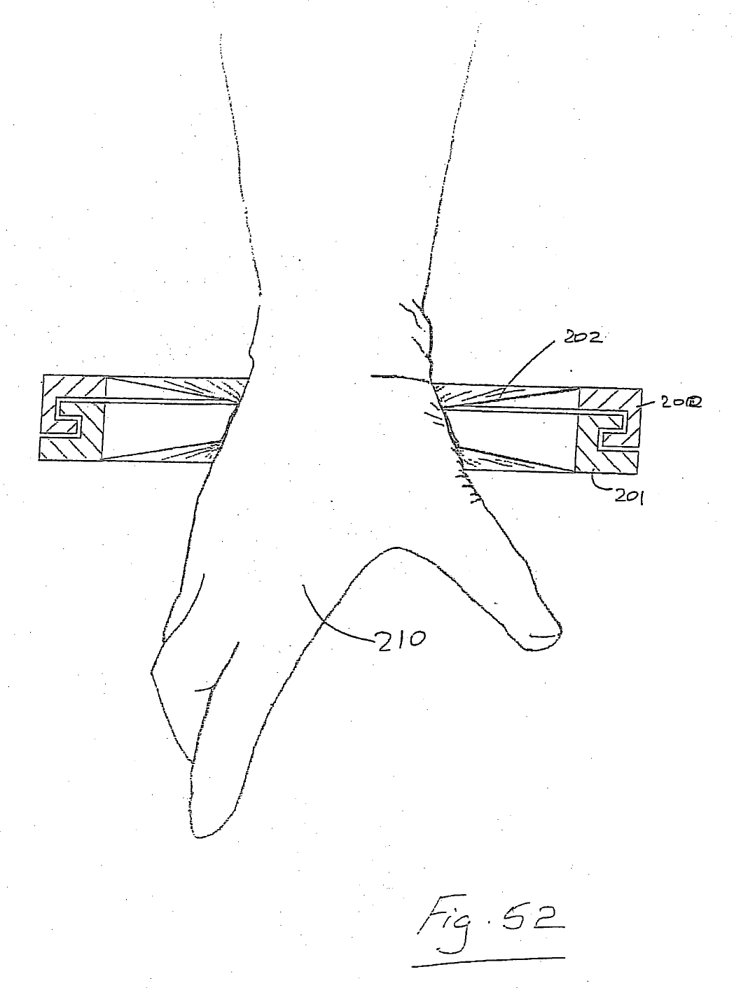

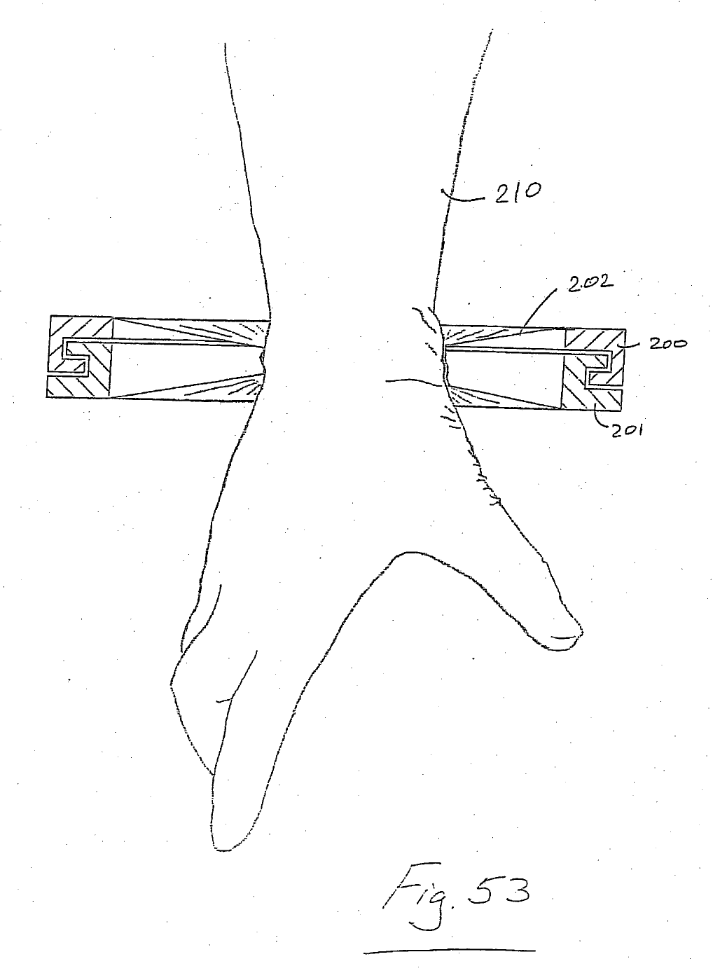

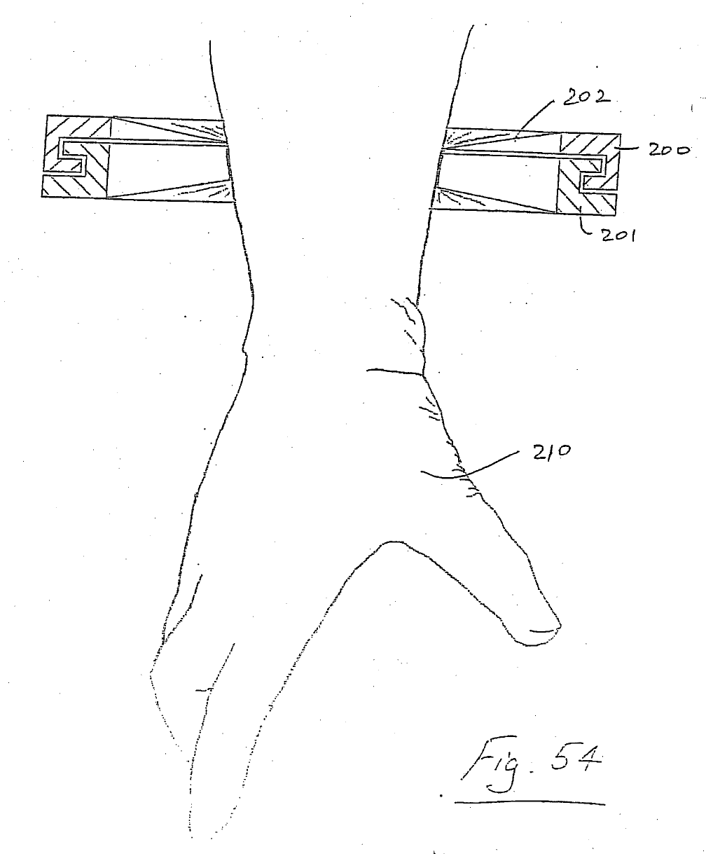

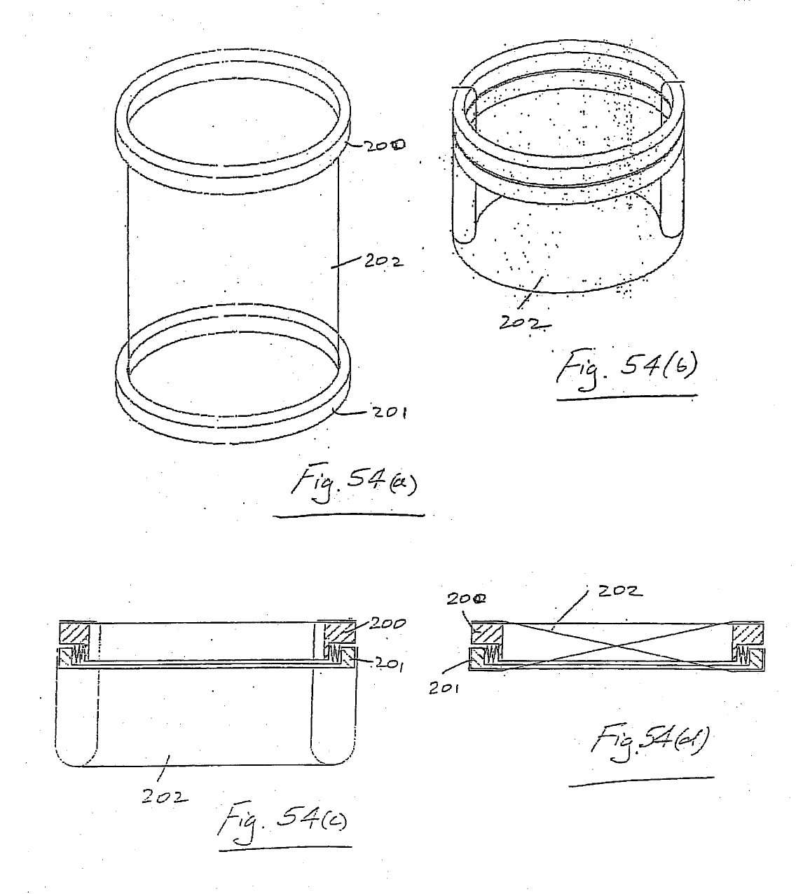

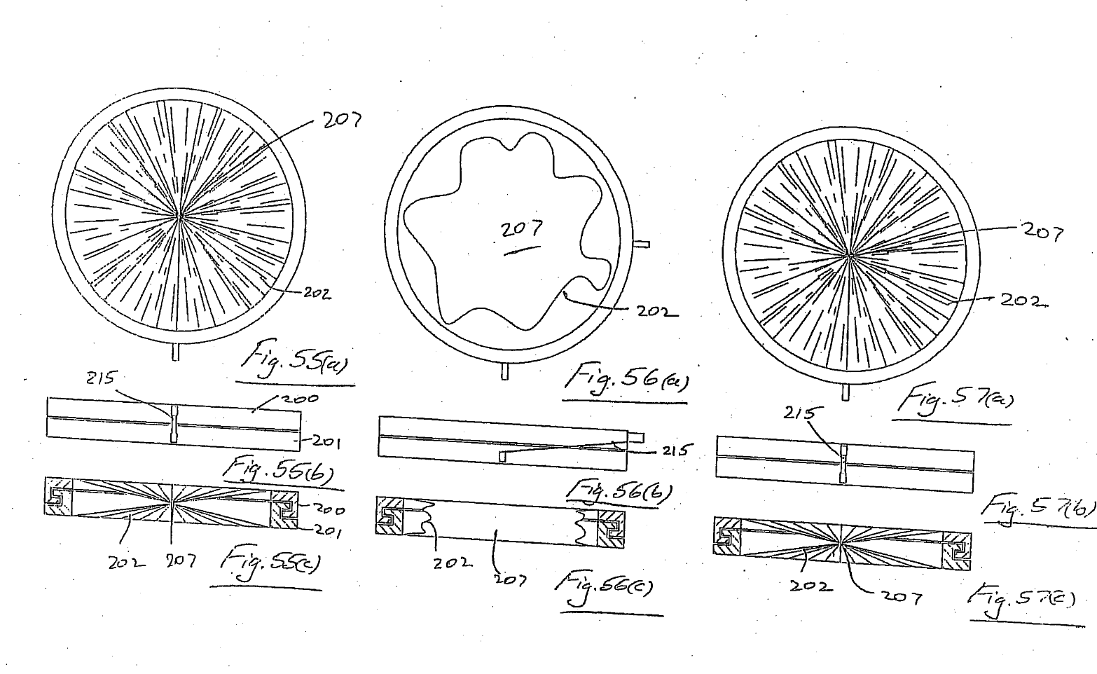

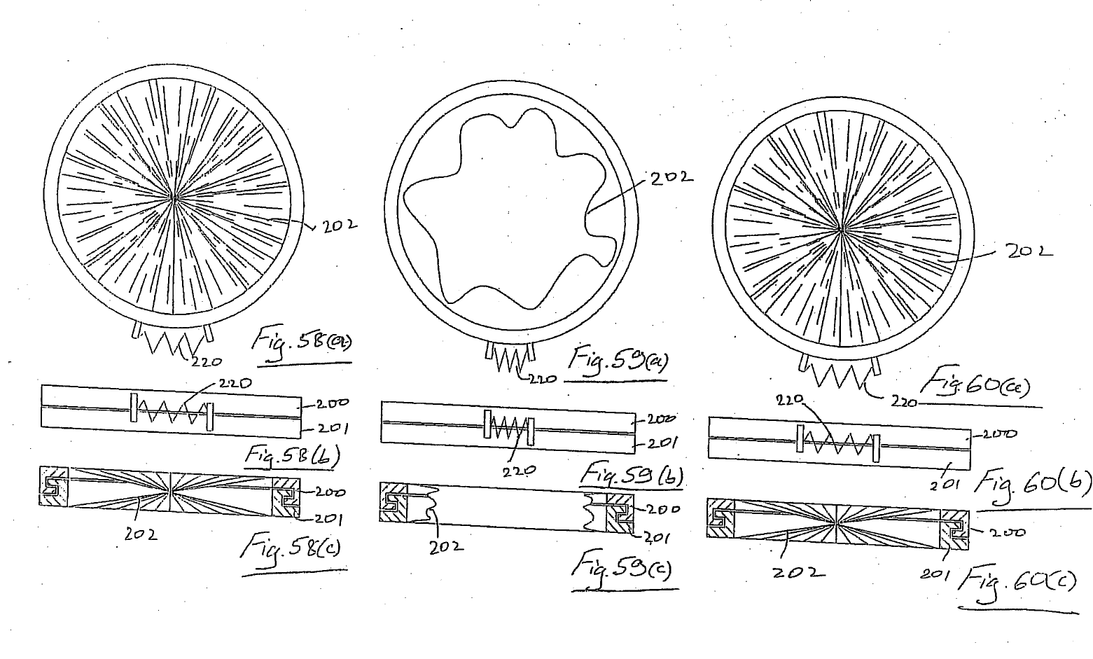

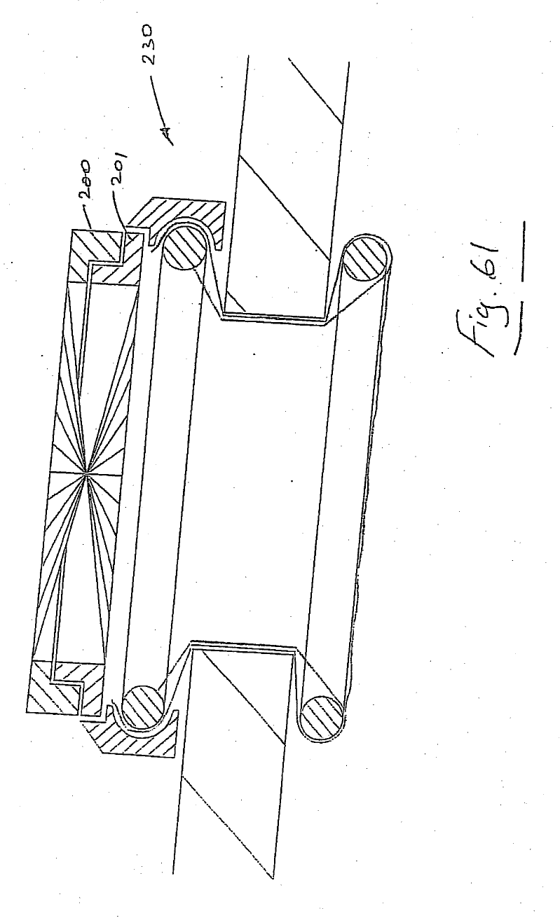

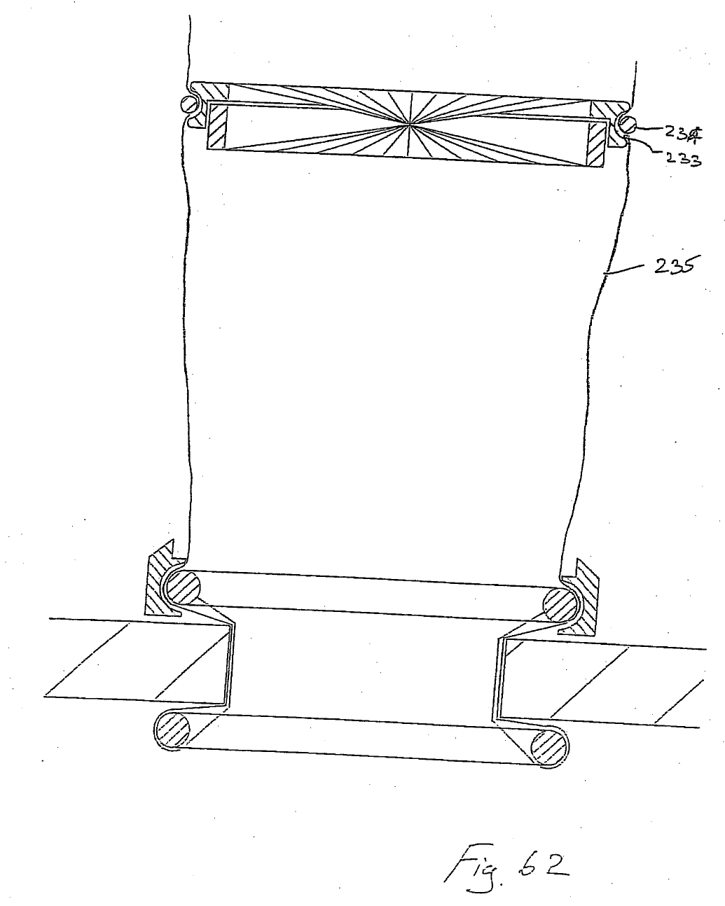

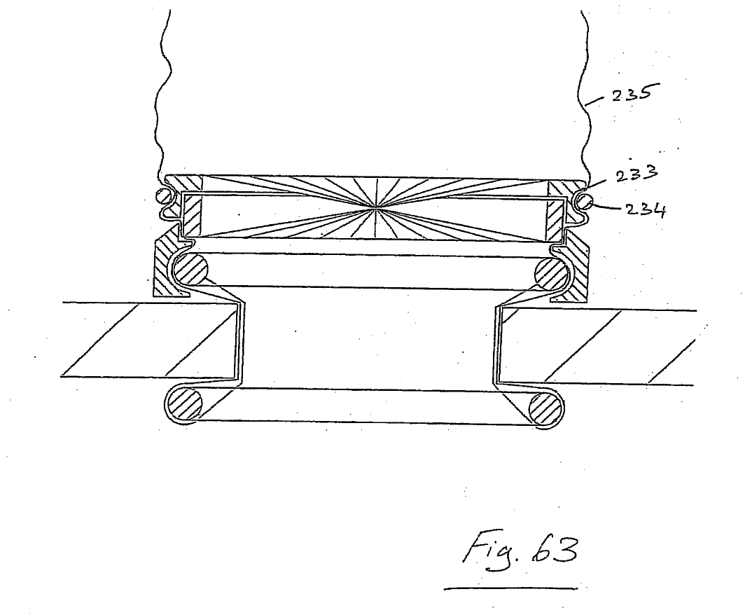

To configure the retractor device according to the invention a sleeve 2 is first provided with the gripping ring 15 fixed at one end and the proximal ring 4 fixed at the other end [FIGS. 3, 5]. The distal ring 3 is then placed over the sleeve 2 as illustrated in FIGS. 4 and 6. The gripping ring 15 is then used to manipulate the sleeve 2 so that the sleeve 2 is folded back on itself into the configuration of FIGS. 1 and 2 in which the gripping ring 15 is uppermost. The sleeve extends from the proximal ring 4 and the distal ring 3 is contained between inner and outer layers 2a, 2b of the sleeve 2. The device is now ready for use.

The resilient distal ring 3 is scrunched up and inserted through the incision 6 with the distal end 5 of the sleeve 2 as illustrated in FIG. 4. The sleeve 2 is then pulled upwardly in the direction of the arrows A in FIGS. 8 to 10. On pulling of the sleeve 2 upwardly the outer layer 2b is pulled up while the inner layer 2a is drawn around the proximal ring 3. This results in shortening the axial extent between the proximal ring 4 and the distal ring 3, tensioning the sleeve and applying a retraction force to the margins of the incision 6. The system appears to be self locking because we have observed that when tension is applied to the sleeve 2 and the pulling force is released the rings 3, 4 remain in position with a retraction force applied. Frictional engagement between the layers of the sleeve in this configuration may contribute to this self locking.

As the incision is being retracted the margins are also protected by the sleeve. On retraction, an access port is provided, for example for a surgeon to insert his hand and/or an instrument to perform a procedure.

Excess sleeve portion 20 outside the incision may, for example, be cut-away.

The retractor is suitable for a range of incision sizes and is easily manufactured. It is also relatively easy to manipulate, in use.

Referring now to FIGS. 11 to 19 there is illustrated another device 50 according to the invention which is similar to the device described above with reference to FIGS. 1 to 10 and like parts are assigned the same reference numerals. In this case the device comprises a guide member 51 for the proximal ring 4. The guide member 51 is in the form of an annular ring member with an inwardly facing C-shaped groove 52 which is sized to accommodate the ring 4 as illustrated. The outer layer of the sleeve 2 is interposed between the ring 4 and the guide 51 to further control the pulling of the sleeve and thereby further controlling the application of the retraction force. The guide 51 also assists in stabilising the proximal ring 4. The use of the device 50 is illustrated in FIGS. 12 to 15 is similar to that described above.

Referring to FIG. 16, it will be noted that in one case the excess sleeve portion 20 may be cut-away.

Referring to FIG. 17, in this case the excess sleeve portion is inverted 60 into the incision. In this configuration it may act as an organ retractor, or provide the surgeon with an open tunnel to work in.

Referring to FIGS. 18 and 19 in this case the excess sleeve portion is twisted to form an iris diaphragm valve 65.

In the embodiment illustrated in FIGS. 20 to 22 a device 70 according to the invention has an integral seal/valve 71. The device 70 is similar to that described above with reference to FIGS. 11 to 19 and like parts are assigned the same reference numerals. In this case the guide member 50 has an outer groove 75 to receive the gripping ring 15 as illustrated in FIGS. 21. The excess sleeve portion 20 is folded out and down and the gripping ring 15 is engaged in the groove 75 to provide an air tight seal. In this configuration the excess sleeve may be inflated through an inflation port 76 [FIG. 22] to provide an integral access valve 71. The valve may be used to sealingly engage a hand, instrument or the like passing therethrough. The inflated sleeve portion defining the valve is evertable on passing an object therethrough.

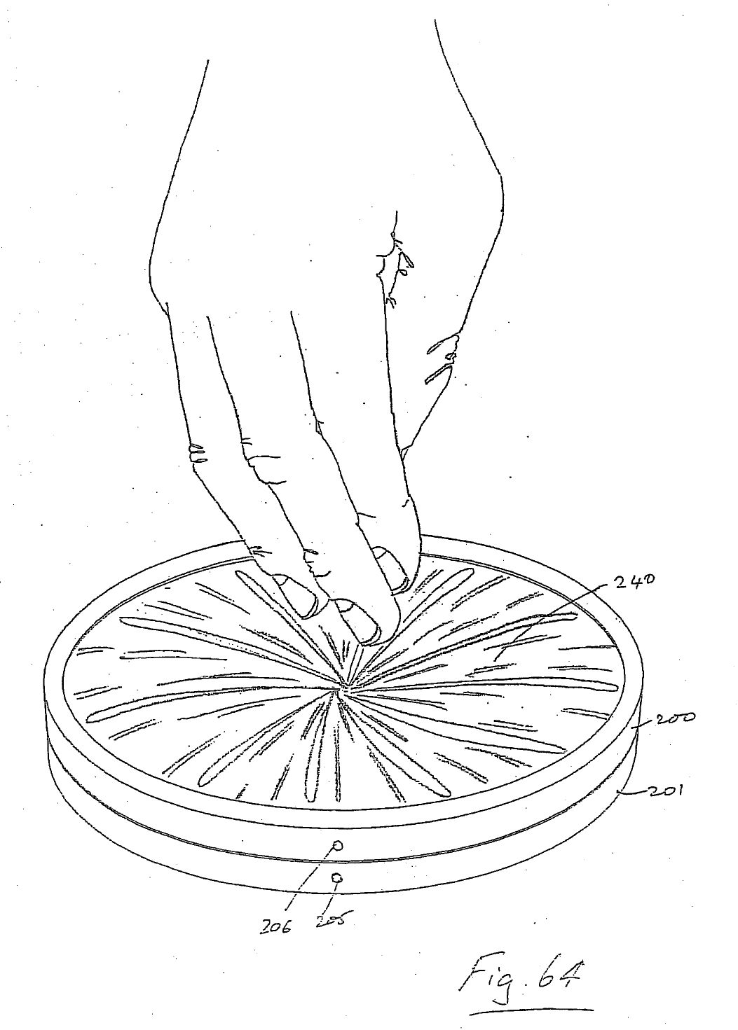

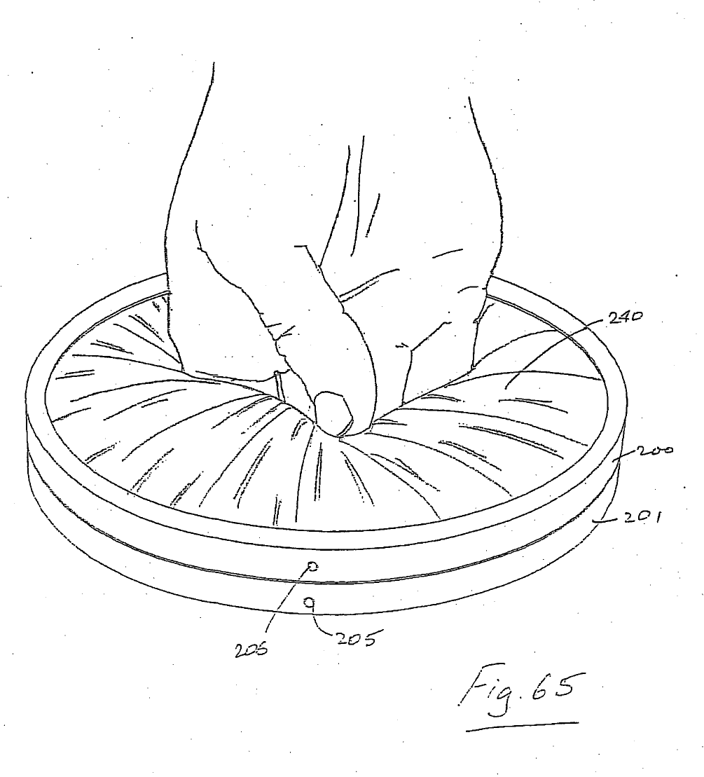

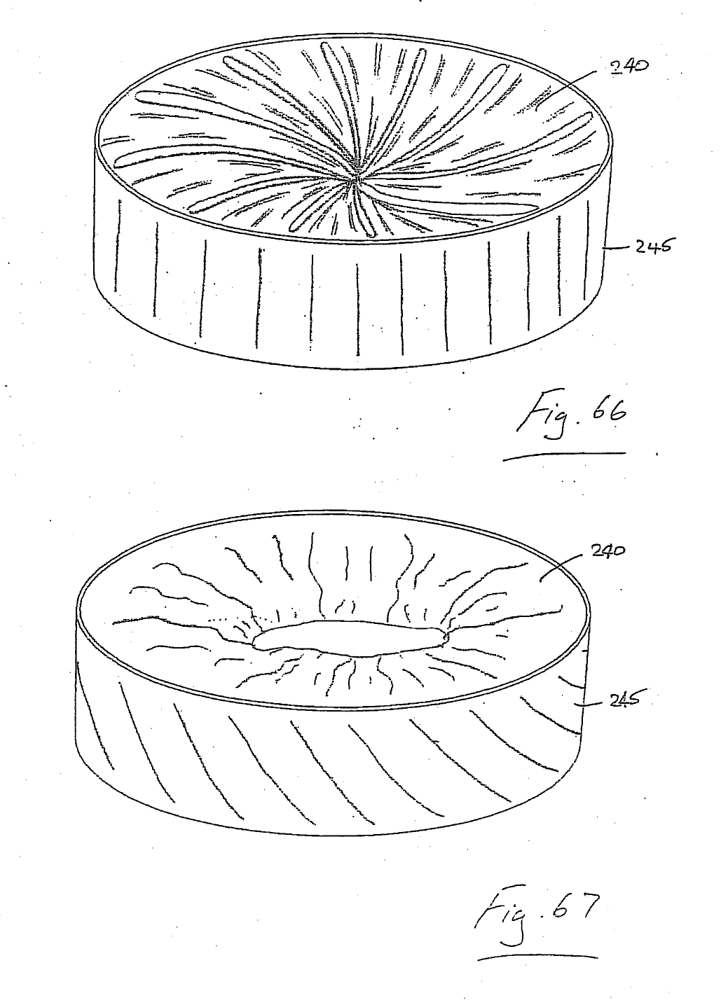

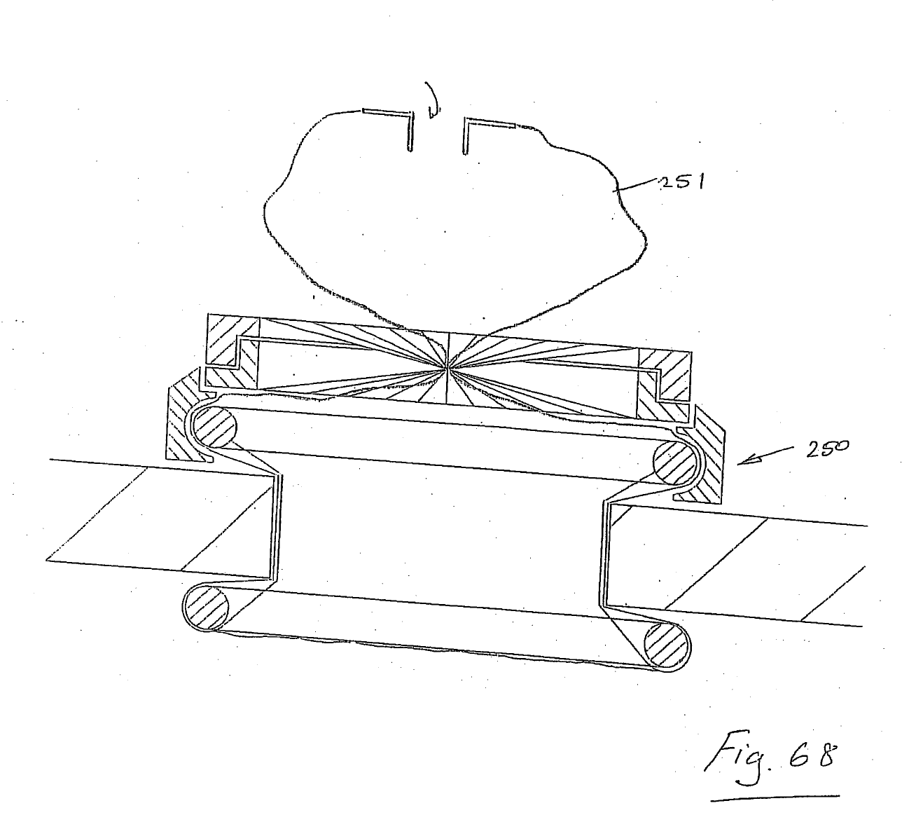

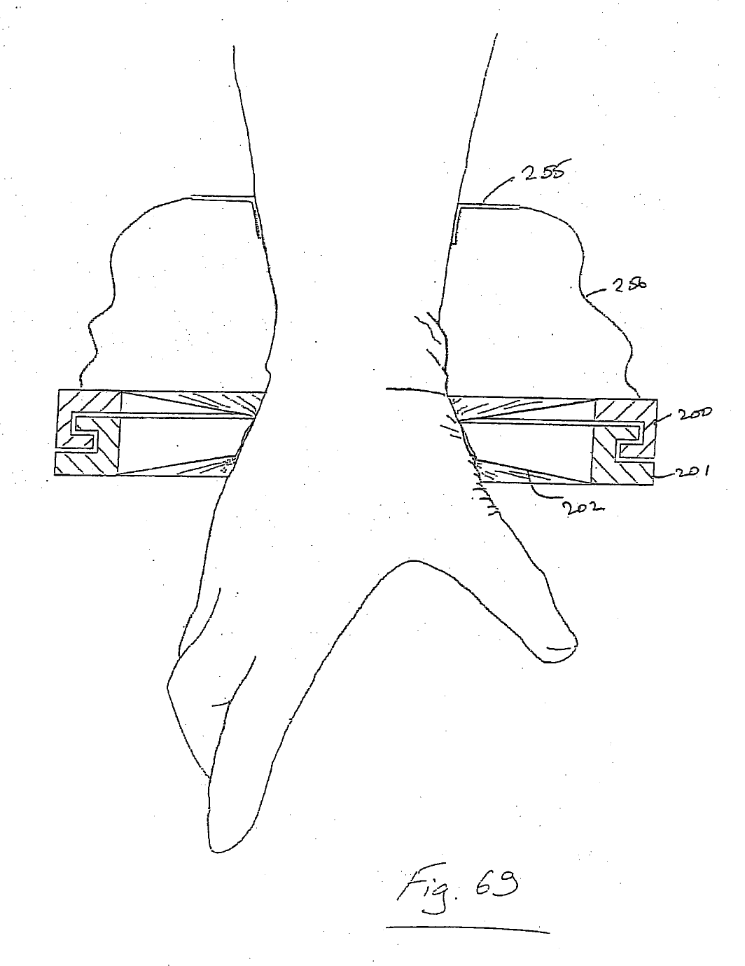

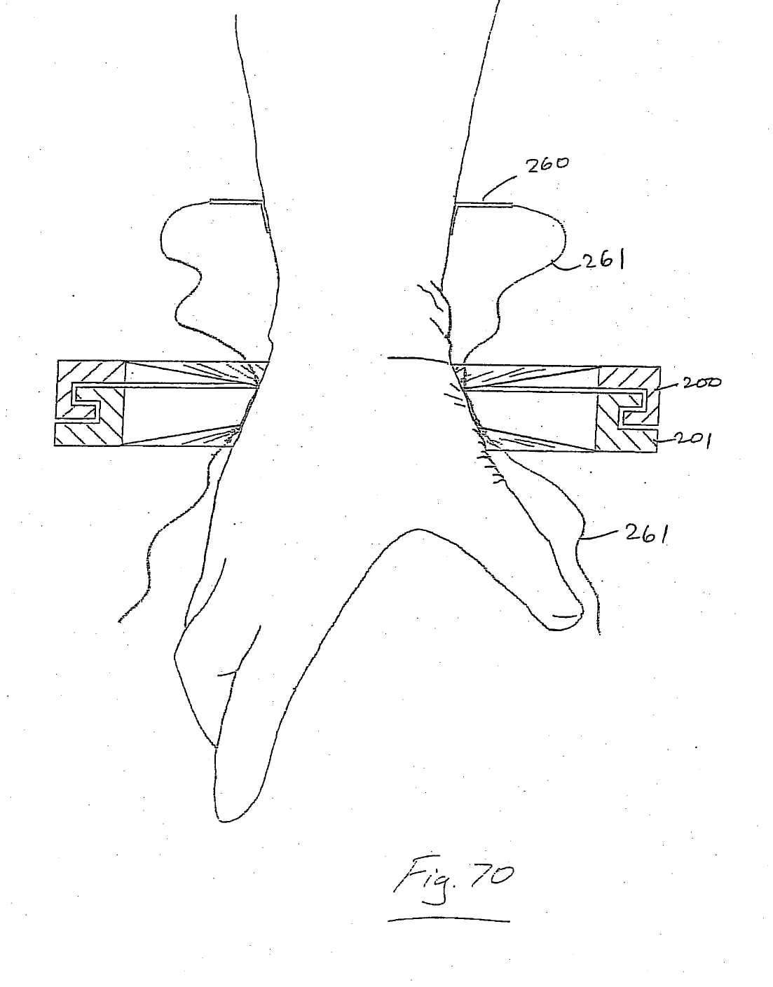

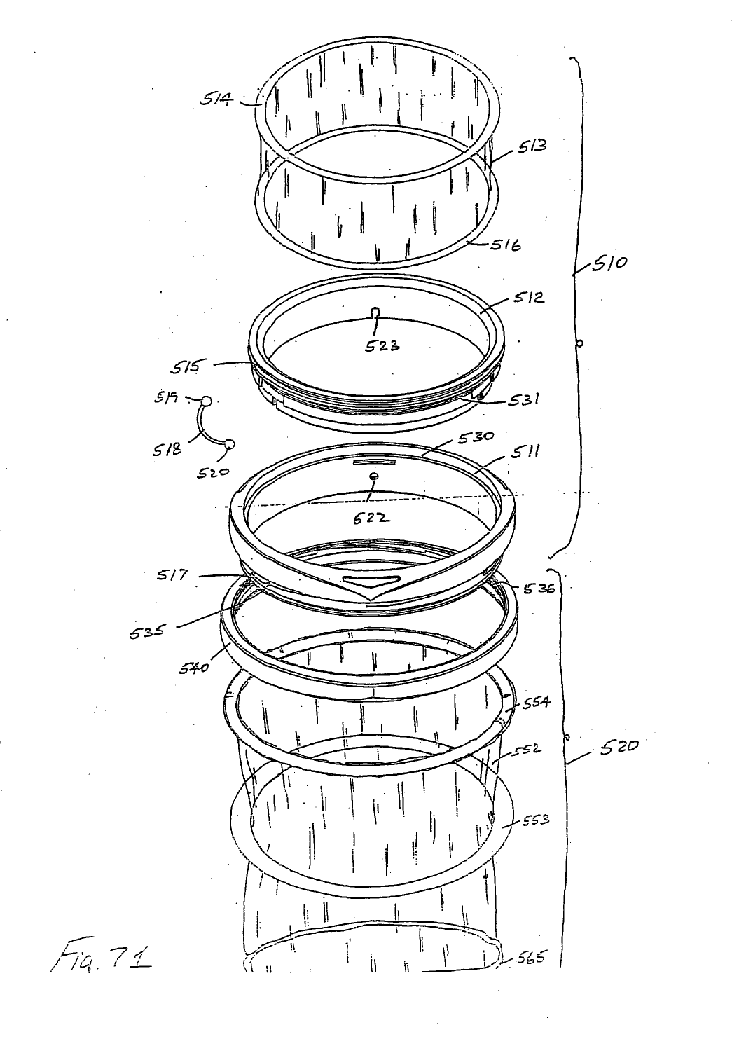

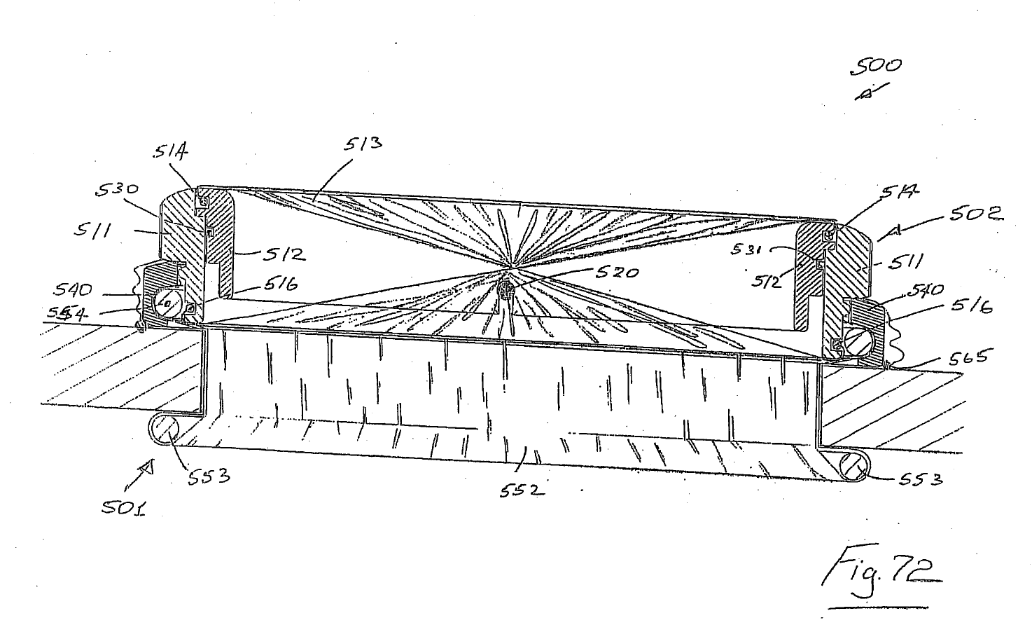

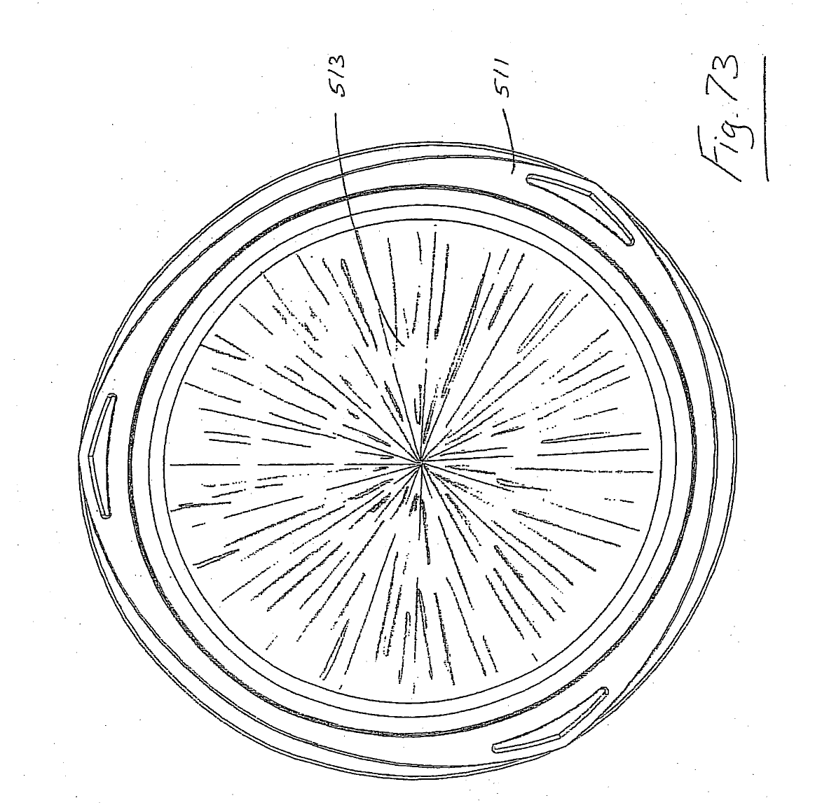

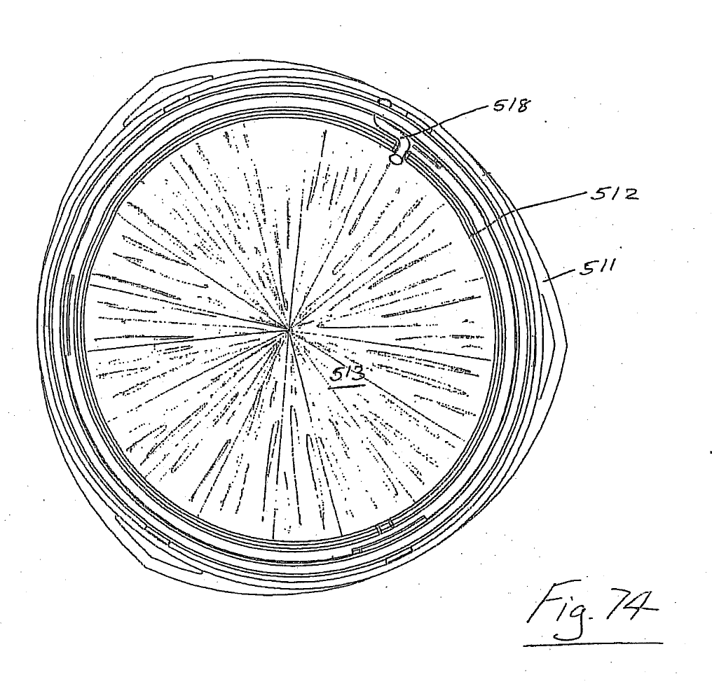

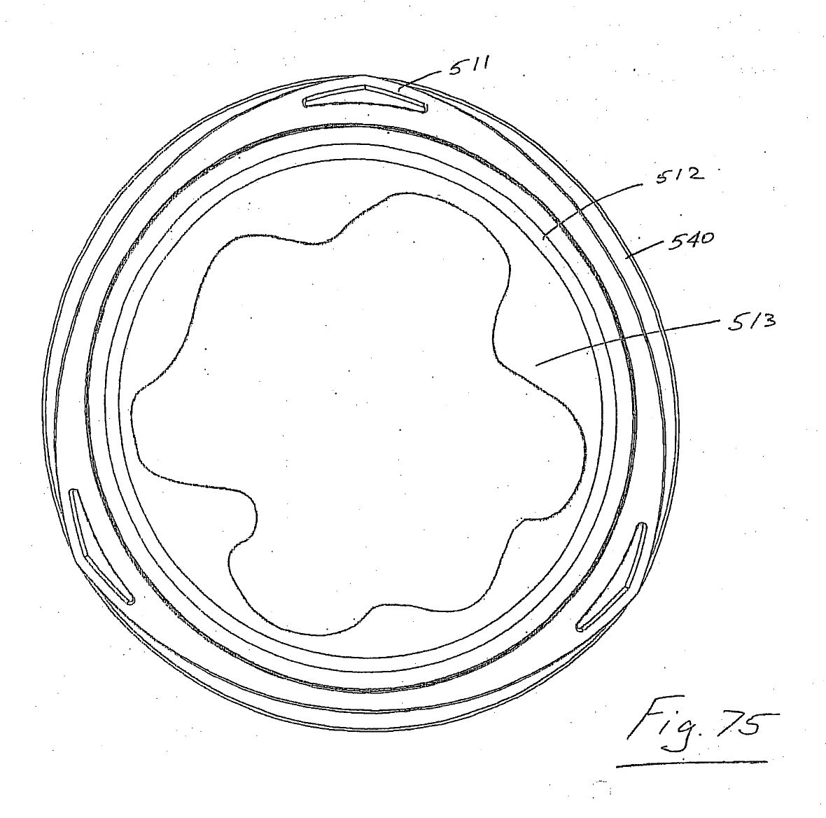

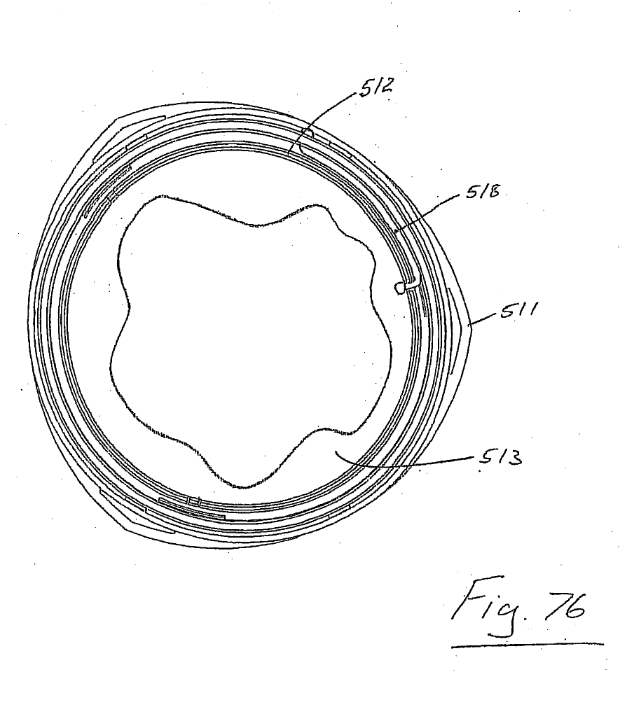

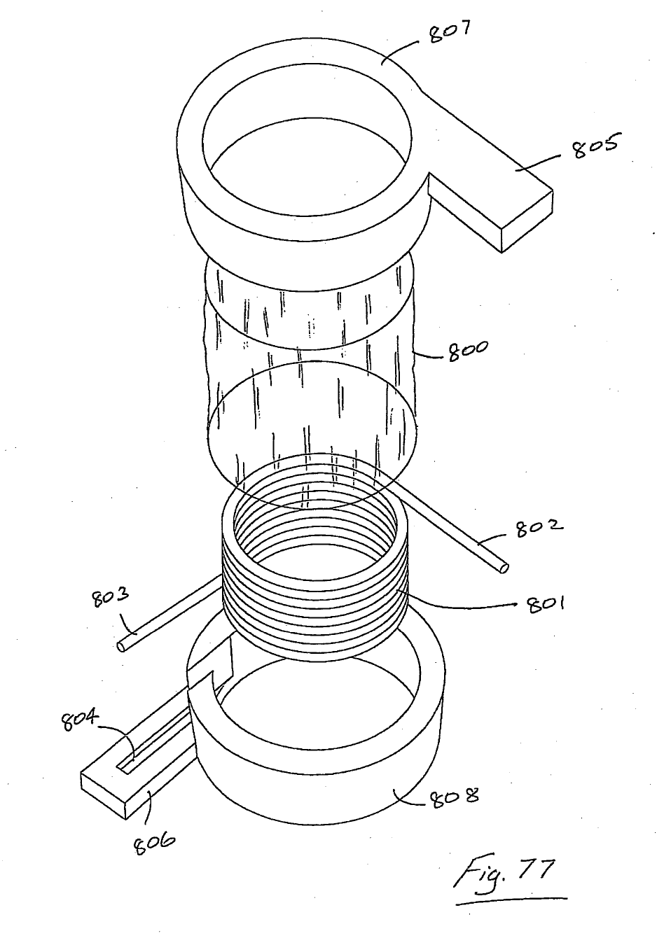

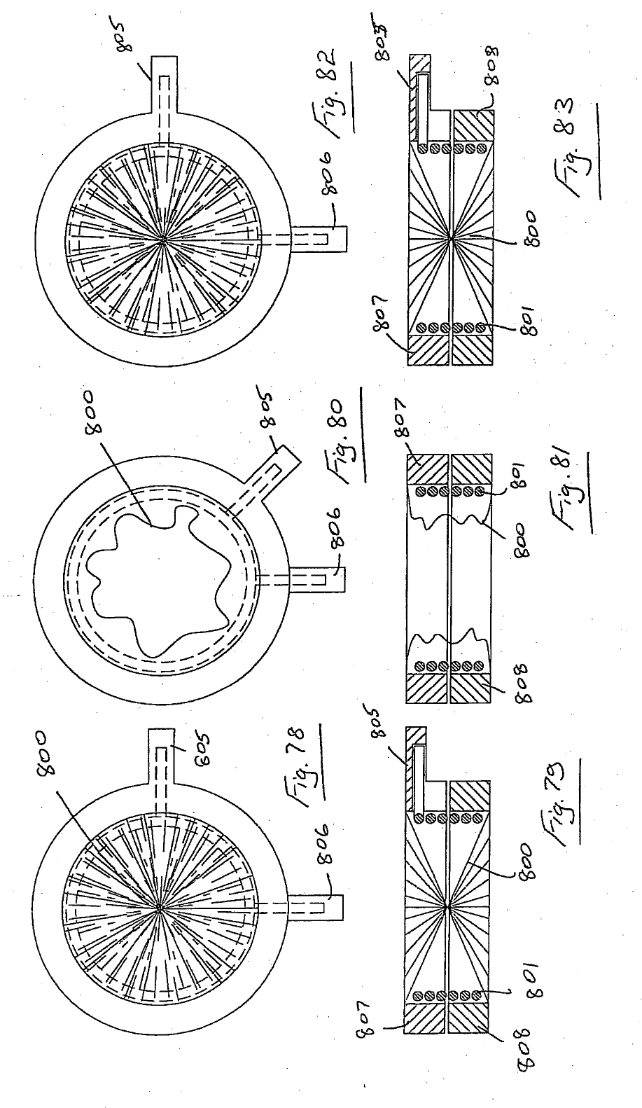

Referring to FIGS. 23 to 28 there is illustrated another retractor 80 according to the invention which is similar to the retractors described above and like parts are assigned the same reference numerals. In this case the retractor 80 has a release mechanism which in this case is provided by a release cord or ribbon 81 which is coupled at one end 82 to the inner ring 3 and terminates in an outer free end 83 which may be grasped by a user. The ribbon 81, on assembly, is led through the gap between the proximal ring 4 and the outer guide member 51 so that it is positioned between the ring 4 and the guide member. The ribbon 81 facilitates release of the self locked sleeve in the in-use configuration sited in an incision. Pulling on the ribbon 81 pulls on the inner ring 3, allowing the ring 3 to be released from the inner wall of the incision to thereby release the device. The flexibility of the ring 3 facilitates this movement.

The advantage of this arrangement is that a user can readily release the device from its self locked retracting configuration.

Referring to FIGS. 29 to 33 there is illustrated another device 90 according to the invention in which parts similar to those of the devices described above are assigned the same reference numerals. In this case the device 90 has a lower guide ring 51 for the proximal ring 4 and an outer guide assembly provided by an upper guide ring 91 and a second proximal ring 92 between which the sleeve 2 is led. The device is used to first retract an incision as described above. During this phase the outer guide assembly is conveniently external of the guide member 51 and proximal ring 4. Indeed, it may be completely detached from the sleeve 2 and subsequently coupled to the sleeve 2 at an appropriate stage such as when the incision is retracted as illustrated in FIG. 30. The outer guide assembly is then moved downwardly towards the incision as illustrated in FIG. 31. This may be achieved while pulling the sleeve 2 upwardly. When the guide assembly is adjacent to the guide member 51 excess sleeve length may be severed as illustrated in FIG. 32. By twisting the guide assembly relative to the guide member 51 the sleeve 2 is twisted, closing down the lumen of the sleeve 2 and forming an iris type access valve 95 as illustrated in FIG. 33. In this way a sealed access port is provided for hand and/or instrument access through the incision.

It will be appreciated that while reference has been made to an incision made by a surgeon the device may be applied for retraction of any opening such as a body opening.

Referring to FIGS. 34 and 35 there is illustrated another retractor device 100 according to the invention which is similar to the device of FIGS. 29 to 33 and like parts are assigned the same reference numerals. In this case a releasable lock is provided to maintain the access valve 95 closed. For interlocking, in this instance the upper guide ring 91 is an interference fit with the lower guide ring 51. Various other locking arrangements may be used such as a screw threaded or bayonet type engagement, magnets, clips and the like.

Referring to FIGS. 36 to 41 there is illustrated another retractor device 110 according to the invention which is similar to the device of FIGS. 29 to 33 and like parts are assigned the same reference numerals. In this case the device incorporates a biasing means to bias an integral valve into a closed position. The biasing means is in this case provided by a coil spring 111 which is located around the sleeve between the guide rings 51, 91. In use, the device is used in a similar manner to the device of FIGS. 29 to 33 except that on movement of the upper guide ring 91 downwardly the spring 111 also moves downwardly towards the lower guide ring 51, initially into the position illustrated in FIG. 38. Excess sleeve material may be removed at this stage. The spring 111 is tensioned as the upper ring 91 is rotated while pushing the upper ring 91 downwardly. The sleeve material between the two rings 51, 91 is twisted, forming an iris type valve 112 as illustrated in FIG. 39. To open the valve 112 to pass an object such as an instrument, hand, arm or the like therethrough a downward force may be applied to push the upper ring 91 towards the lower ring 51 against the biasing of the spring. This configuration is illustrated in FIG. 40. When the object is inserted the upper ring member 91 is released, allowing the valve to close around the object. The operation of the device 110 will be readily apparent from FIGS. 41(a) to 41(d). In FIG. 41(a) the valve 112 is illustrated in a closed resting configuration. FIG. 41(b) shows the application of a downward force to open the valve 112. An object such as an instrument 113 is shown inserted through the open valve 112 in FIG. 41(c). In FIG. 41(d) the downward pressure on the upper ring 91 is released allowing the valve 112 to close around the object 113.

Referring now to FIGS. 42 to 45 there is illustrated another device 120 according to the invention which has some aspects similar to the device of FIGS. 11 to 18 and like parts are assigned the same reference numerals. In this case the device has a lip seal 121. The lip seal 121 is provided by a membrane with a central aperture 122 through which an object 123 such as an instrument is passed. The lip seal 121 is located on the sleeve 2 proximally of the guide ring 51 such that a proximal flexible sleeve section 125 is provided. This sleeve section 125 is very useful in facilitating offset movements of the object 123 as illustrated in FIG. 45. The sleeve section 125 accommodates movement of the object 123 whilst maintaining sealing engagement between the lip seal 121 and the object 123. It will be appreciated that this feature, as with several other features described above may be utilised in association with other constructions of wound protector/retractors and access ports generally other than those illustrated in the drawings.

Referring to FIGS. 46 to 48 there is illustrated another device 130 according to the invention which has some features similar to those of FIGS. 11 to 15, like parts being assigned the same reference numerals. In this case the sleeve has a proximal section external of the wound when the device is in the retracting configuration. This proximal sleeve section comprises a first portion 131 extending from the guide ring 51 and a second portion 132 extending from the first portion 131. The second portion 132 is defined between two spaced-apart iris rings 134, 135. It will be noted that the iris rings 134, 135 have engagement features such as projections and grooves for interengagement on assembly. The iris ring 134 also has an engagement element, in this case provided by a groove 137 for engagement on assembly with a corresponding engagement element of the guide ring 51 which in this case is provided by a projection 138.

The device is fitted as described above to retract an incision, leaving the first and second sleeve portions 131, 132 extending proximally. The first sleeve portion 131 is redundant and can be removed or scrunched up on assembly of the first iris ring 134 to the guide ring 138 as illustrated in FIG. 48. The second or upper iris ring 135 is then rotated to twist the sleeve section 132 to form an iris-type seal as illustrated in FIG. 49. The iris ring 135 is engaged with the iris ring 134 as illustrated to maintain the valve closed.

Referring to FIG. 49(a) there is illustrated another device 140 according to the invention which has some aspects similar to the device of FIGS. 46 and like parts are assigned the same reference numerals. In this case the iris rings 134, 135 are used to form an iris valve 141 which is proximally spaced from the guide ring 51 and a flexible sleeve section 142 is thereby provided between the iris 141 and the guide ring 51. This sleeve section 142 can act as a flexible cannula wall to permit sealed access of a cannula whilst facilitating lateral movement of the cannula somewhat as illustrated in FIGS. 44 and 45.

Referring to FIGS. 49(b) to 49(i) there is illustrated a device according to the invention 150 comprising a first ring element 200, a second ring element 201 and a sleeve 202 of pliable material with a first end mounted to the first ring element 200 and a second end mounted to the second ring element 200. For ease of reference the ring elements 200, 201 have associated location markings 205, 206 respectively. The sleeve 202 is twisted and has a normally closed access opening 207 and the sleeve is movable on insertion of an object such as a surgeon's hand/arm 210 or an instrument through the access opening 207.

As will be described in more detail below a biasing means is provided to bias the sleeve to close the access opening 207. The biasing may be provided by pre-tensioning the sleeve, or by using a separate spring element.

Referring to FIG. 49(j) there is illustrated another device 160 which is similar to the device of FIGS. 49(d) to (i) and like parts are assigned the same reference numerals. In this case the device has a lip-type seal 161. Another device 165 with a different type of lip seal 162 is illustrated in FIG. 49(k).

Referring to FIGS. 50 to 57(c) there is illustrated an access port according to the invention for use in surgery comprising a first ring element 200, a second ring element 201 and a sleeve 202 of pliable material with a first end mounted to the first ring element 200 and a second end mounted to the second ring element 200. For ease of reference the ring elements 200, 201 have associated location markings 205, 206 respectively. The sleeve 202 is twisted and has a normally closed access opening 207 and the sleeve is movable on insertion of an object such as a surgeon's hand/arm 210 or an instrument through the access opening 207.

A biasing means is provided to bias the sleeve 202 to close the access opening 207. The biasing may be provided by pre-tensioning the sleeve, or by using a separate spring element. In this case the spring element 215 is a strip of elastic material 215 which is mounted at one end to the first ring 201. The elastic strip 215 causes the rings to be biased into a rest position at which the opening 207 is closed. On insertion of an object such as a surgeon's hand the entry force acts against the biasing of the elastic strip 215 and the rings 200, 201 rotate relative to one another as evidenced by the locator marks 205, 206. However, the opening is only sufficient to allow a specific sized object such as a hand and forearm to be inserted through the sleeve whilst maintaining continuous sealing engagement between the sleeve and the object such as a surgeon's hand/forearm, thus ensuring that there is no gas leakage and maintaining pneumoperitoneum. The device is very easily manufactured and, most importantly, is extremely easy for a surgeon to use, as a sealed access port is provided through which a surgeon can easily insert his arm and forearm. It will be noted that the biasing ensures that the access opening substantially exactly matches the contours of the inserted object such as a hand/forearm and automatically opens and closes as required.

In another embodiment, as illustrated in FIGS. 58(a) to 60(c), the spring element may be a coiled spring 220 which normally biases the rings in such a way as to close the opening.

Referring to FIG. 61 the hand access device of FIGS. 50 to 60 is shown mounted to a retractor 230 such as a retractor as described above.

Referring to FIGS. 62 and 63 the access device is shown being mounted to another type of retractor 240. In this case the first ring element 200 has a circumferentially extending groove 233 and an associated ring 234 with a retractor sleeve section 235 accommodated therebetween to permit sliding action of the access device relative to the retractor sleeve section 235.

It will, however, be appreciated that the access devices of the invention can be used with any suitable retractor or other similar device.

Referring to FIGS. 64 and 65 there is illustrated another access device which is similar to the device of FIGS. 50 to 57 except that in this case the biasing to close the access port is provided by pre-tensioning the sleeve 240 and the surgeon, on insertion of an object such as his hand/arm acts to overcome the tension in the sleeve sufficient to allow hand insertion whilst still maintaining sealing engagement to the object such as the surgeon's hand/arm. This configuration will also be apparent from FIGS. 66 and 67. The twisted sleeve defining an iris is shown in FIG. 66 with a strong outer resilient material 245. As a surgeon inserts his hand the twist in the sleeve 202 is transferred to the outer resilient material 245 with the applied force. In FIG. 67 the hand is removed for clarity, in reality on removal of the hand the system will revert to the closed configuration of FIG. 66.

Referring now to FIG. 68 there is illustrated an assembly of a access device of the invention with a retractor 250 having an excess retractor sleeve section 251 provided with an outer lip seal 252 for sealing engagement to the arm of a surgeon. The excess retractor sleeve section may be used to externalise an organ during a surgical procedure. In FIG. 69 a lip seal 255 is provided in a sleeve section 250 mounted to the ring element 200. In FIG. 70 a lip seal 260 is provided on a separate sleeve section 261.

Referring to FIGS. 71 to 76 there is illustrated an assembly 500 of the invention which comprises a retractor 501 and an iris valve 502 releasably mounted to the retractor 501. The retractor 501 is similar to the retractors described above such as with reference to FIGS. 1 to 10. The iris valve 502 is similar to the iris valves described above such as with reference to FIGS. 50 to 57(c).

The iris comprises the components within the chain bracket 510 in FIG. 71 and the retractor comprises the components within the chain bracket 520 in FIG. 71.

The iris 502 comprises a fixed outer iris ring member 511 and an inner rotatable ring member 512. The inner ring member 512 is in this case a snap fit and is free to rotate relative to the outer ring member 511. The snap fit engagement is through an annular rib 530 on the outer ring member 511 and a corresponding annular groove 531 in the inner ring member 512. A flexible iris-forming sleeve 513 extends between the inner and outer ring members 511, 512. The sleeve 513 has a first elasticated ring or band 514 at one end for anchoring in a corresponding engagement channel 515 in the inner ring member 512 and a second elasticated band 514 at the other end for anchoring in a corresponding engagement channel 517 in the outer ring member 511. Thus, one end of the iris-forming sleeve 513 is anchored to the movable ring member 512 and the other end is anchored to the fixed ring member 512 so that rotation of the ring member 512 relative to the fixed ring member 511 will result in twisting or untwisting of the sleeve, forming an iris valve. The iris valve is biased into a normally closed position (FIGS. 72 to 74) by a spring which in this case is in the form of a strip of elastic material 518 having enlarged head portions 519, 521 at the ends thereof for location and engagement of one end of the spring 518 in a spring locating hole 522 in the fixed ring member 511 and for location and engagement of the other end of the spring 518 in a spring locating slot 523 in the rotatable ring member 512. The spring 518 biases the iris-forming sleeve 513 into the normally closed position. In insertion of an object such as a surgeons hand, the biasing force of the spring is counteracted causing partial opening of the iris valve whilst still remaining sealing engagement of the iris sleeve with the object passing therethrough. A twisting action of the object as it is being inserted will aid overcoming of the spring biasing action, in some cases. The operation of the iris is described in more detail above.

The iris forming sleeve 513 has a length in the unassembled untwisted configuration of 71 that is preferably less than or equal to the diameter of the sleeve 513. We have found that this is advantageous in optimising the operation of the iris by ensuring full closure of the iris whilst ensuring that excess sleeve material, on twisting, is minimised.

The iris valve 502 is in this case releasably mounted to the retractor 501. Thus, the iris 502 may be used independently of the retractor 501 and vice versa. In this instance the iris valve is screw threadingly engagable with the retractor, the outer ring 511 of the iris having a thread 535 for connection to the retractor 501. The retractor 501 in turn has tabs 536 which project inwardly from a retractor top ring 540 for engagement with the screw thread 535 of the outer ring 511. Any suitable interconnection may be provided.

The retractor 501 comprise a sleeve 552, a distal member provided by a distal ring 553 of resilient material such as an O-ring and a proximal member provided by a proximal ring 554 which may also be an O-ring.

The sleeve 552 is of any suitable material such as of pliable plastics film material and comprises a distal portion 555 for insertion through an incision 556, in this case made in a patient's abdomen 557, and a proximal portion 558 for extending from the incision 556 and outside of the patient.

In this case the distal ring 553 is not fixed to the sleeve 552 but rather the sleeve is led around the ring 553 and is free to move axially relative to the distal ring 553 somewhat in the manner of a pulley. The proximal ring 554 is fixed to the sleeve 552, in this case at the proximal inner end thereof. The sleeve 552 terminates in a handle or gripping portion which in this case is reinforced by a gripping ring 565. As described above with reference to FIGS. 1 to 10, to configure the retractor device according to the invention a sleeve 552 is first provided with the gripping ring 565 fixed at one end and the proximal ring 554 fixed at the other end [FIGS. 3, 5]. The distal ring 553 is then placed over the sleeve 552. The gripping ring 565 is then used to manipulate the sleeve 552 so that the sleeve 552 is folded back on itself into the configuration of FIGS. 1 and 2 in which the gripping ring 565 is uppermost. The sleeve extends from the proximal ring 554 and the distal ring 553 is contained between inner and outer layers of the sleeve 2. The device is now ready for use.

The resilient distal ring 553 is scrunched up and inserted through the incision 556 with the distal end 555 of the sleeve 552 as illustrated in FIG. 4. The sleeve 552 is then pulled upwardly in the direction of the arrows A in FIGS. 8 to 10. On pulling of the sleeve 552 upwardly the sleeve outer layer is pulled up while the sleeve inner layer is drawn around the proximal ring 553. This results in shortening the axial extent between the proximal ring 554 and the distal ring 553, tensioning the sleeve 552 and applying a retraction force to the margins of the incision 556. The system appears to be self locking because we have observed that when tension is applied to the sleeve 552 and the pulling force is released the rings 553, and 554 remain in position with a retraction force applied. Frictional engagement between the layers of the sleeve in this configuration may contribute to this self locking. As the incision is being retracted the margins are also protected by the sleeve. On retraction, an access port is provided, for example for a surgeon to insert his hand and/or an instrument to perform a procedure.

In this instance the sleeve gripping ring 565 is led over the retractor top ring 540 and the gripping ring 565 is retained outside of the top ring 540 as illustrated in FIG. 72. The retractor top ring 540 provides a guide member for the retractor proximal ring 554. The guide member or top ring 540 is in the form of an annular ring member with an inwardly facing C-shaped groove which is sized to accommodate the ring 554 as illustrated. The outer layer of the sleeve 552 is interposed between the ring 554 and the guide 540 to further control the pulling of the sleeve and thereby further controlling the application of the retraction force. The guide 540 also assist in stabilising the proximal ring 554.

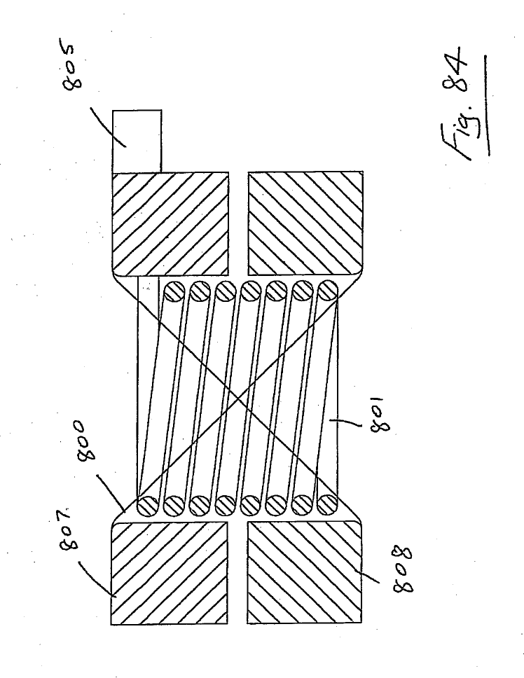

Referring now to FIGS. 77 to 84 there is illustrated a pinch valve for use with the access port of the invention. The pinch valve comprises a flexible cylindrical film sheath 800 which is twisted by a torsion spring 801 to form an iris-type valve. The spring 801 has spring arms 802, 803 at the free ends thereof which are retained within corresponding recesses 804 in finger handle parts 805, 806 of retaining members 807, 808. The valve is normally in the closed position illustrated in Figs. 78 and 79, in which the sleeve 800 is biased by the spring 801 into a closed iris-forming configuration. The handles 805, 806 can be readily gripped by a user with one hand and rotated against the biasing of the spring 801 causing the iris to open as illustrated in FIGS. 80 and 81, ready to receive an object such as an instrument therethrough. When the object has passed through the valve the finger handles 805, 806 are released, causing the iris to close and maintain gas pressure on the patient side of the valve.

Because of the simple and compact open/close arrangement of the finger handles 805, 806, it is possible for a user to open the iris using only an index finger and a thumb of one hand. This is a highly convenient means of operating the valve, especially in the case of passing laparoscopic instruments through the valve.

The access port of FIGS. 77 to 84 may also be used with a further seal such as a lip seal 810 which may be coupled to the top retaining member 807 as illustrated in FIGS. 85 to 88. In these drawings the access port is shown coupled to a retractor 811 located in an incision 813 in the abdomen 812 of a patient to create a low-profile, sealed instrument access port.

The retractor 811 is preferable a retractor of the type described earlier.

In particular the retractor 811 for retracting the sides of the incision 813 comprises a distal O-ring member 1000 for insertion into the incision 813, a proximal O-ring member 1001 for location externally of the incision 813, and a retracting sleeve member 1002 for extending between the O-rings 1000, 1001 to retract the sides of the incision 813 (FIG. 85).

The sleeve 1002 is fixedly attached to the proximal O-ring 1001, is looped distally around the distal O-ring 1000, and extends between the O-rings 1000, 1001 in a two-layer arrangement.

The retractor 811 is particularly suitable for retracting the sides of a laparoscopic incision 813. Generally laparoscopic incisions are retracted to a diameter of less than 40 mm, preferably between 3 mm and 35 mm, and ideally between 5 mm and 12 mm.

As illustrated in FIGS. 87 and 88, the diameter of the retracted laparoscopic incision 813 is substantially equal to the diameter of the laparoscopic instrument 814. This is possible because the walls of the retracting sleeve member 1002 are extremely thin. Thus the minimum amount of space is used up by the walls of the retractor 811 enabling the overall size of the laparoscopic incision 813 to be minimised.

The lip seal 810 provides further sealing for an instrument 814 which may be inserted through the pinch valve and the retractor 811, as illustrated in FIGS. 86 to 88.

In an alternative arrangement illustrated in FIGS. 89 and 90, a lip seal 820 may be connected to the retractor 811, such as by using excess sleeve material 822 from the retractor 811. Other details of this embodiment are described above with reference to FIGS. 77 to 84, and like parts are assigned the same reference numerals.

In another embodiment the access port does not have a secondary seal for the instrument. Such an embodiment is illustrated in FIGS. 91 and 92. Basically this version involves a retractor 811, with a pinch valve arrangement as described above with reference to FIGS. 77 to 74, attached directly thereto.

In some of the embodiments described above a valve is mounted directly to a retractor base 811. It is possible to provide a flexible coupling between the retractor 811 and the valve. For example, as illustrated in FIGS. 93 and 94, such a flexible coupling is provided by a length of flexible sleeve 830 extending between the retractor 811 and the valve 829. The flexible sleeve 830 may be formed by excess retractor sleeve material attached to the valve 829. The flexible nature of the sleeve 830 accommodates movement of the valve 829 relative to the retractor 811 while maintaining the gas-tight sealed coupling.

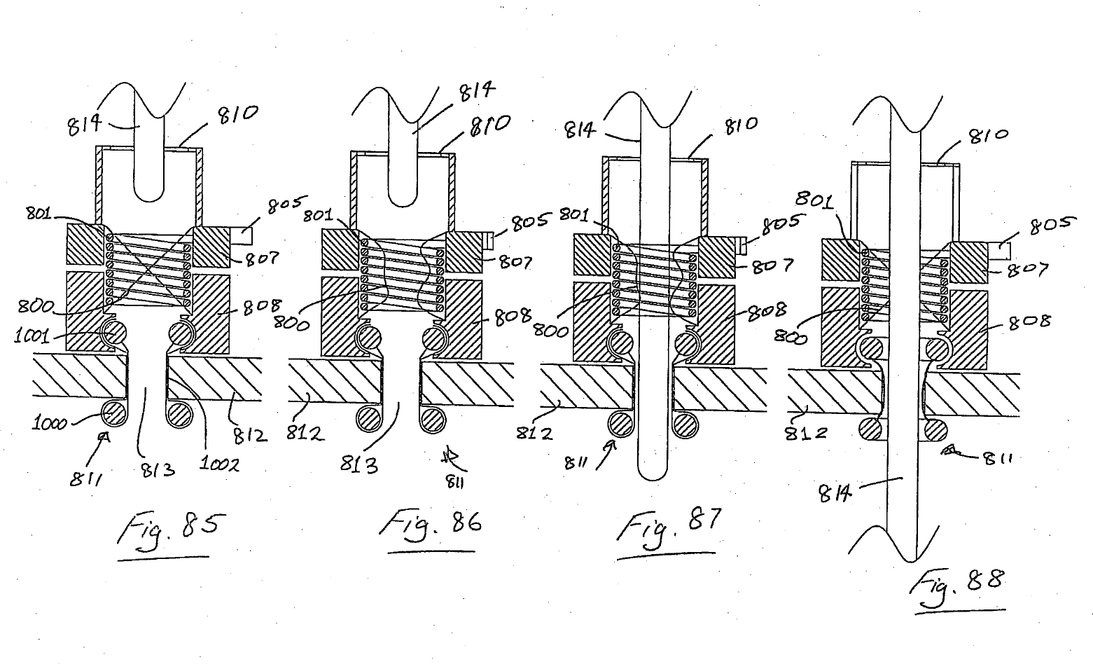

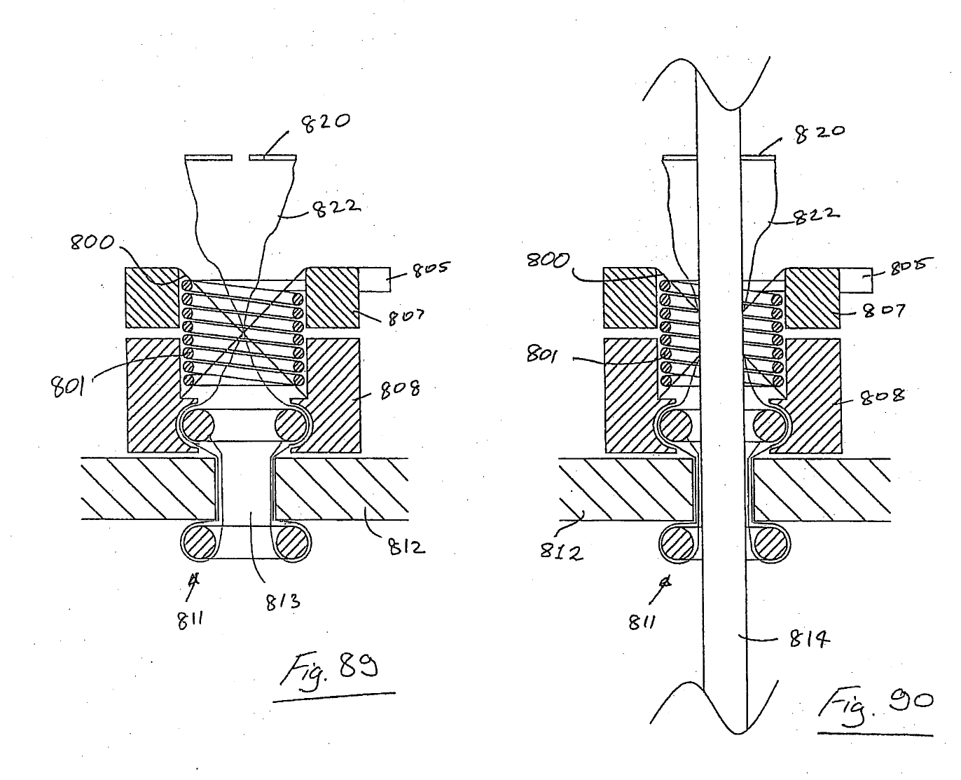

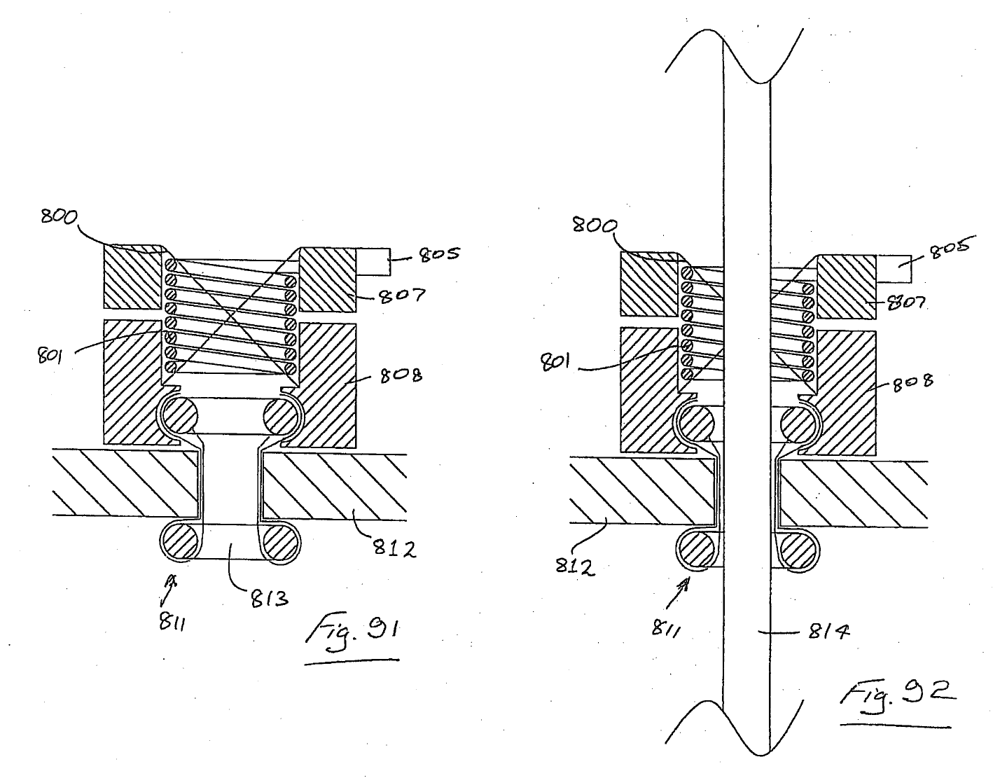

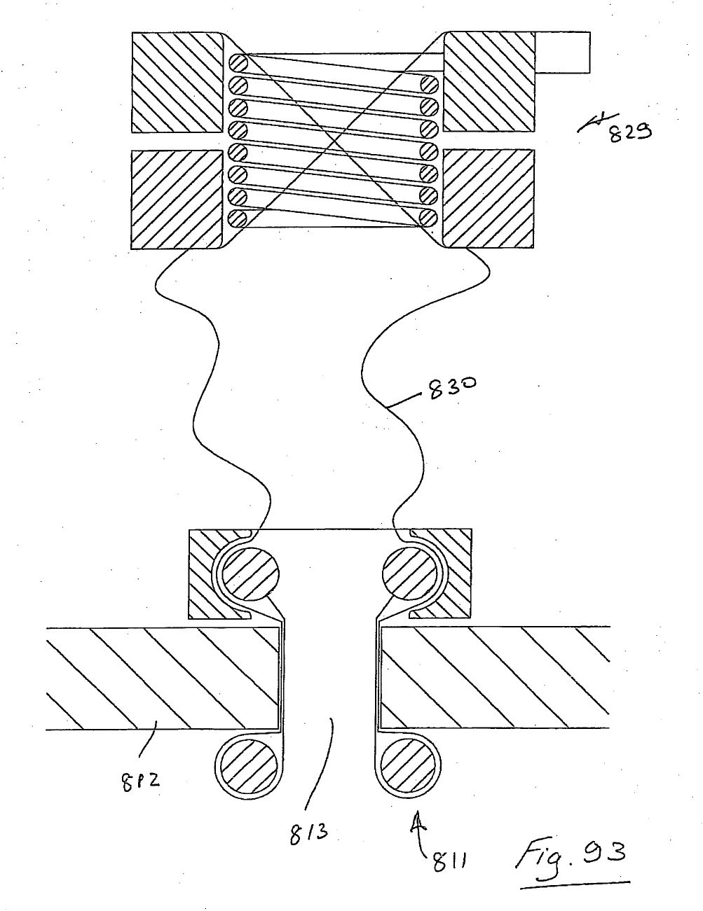

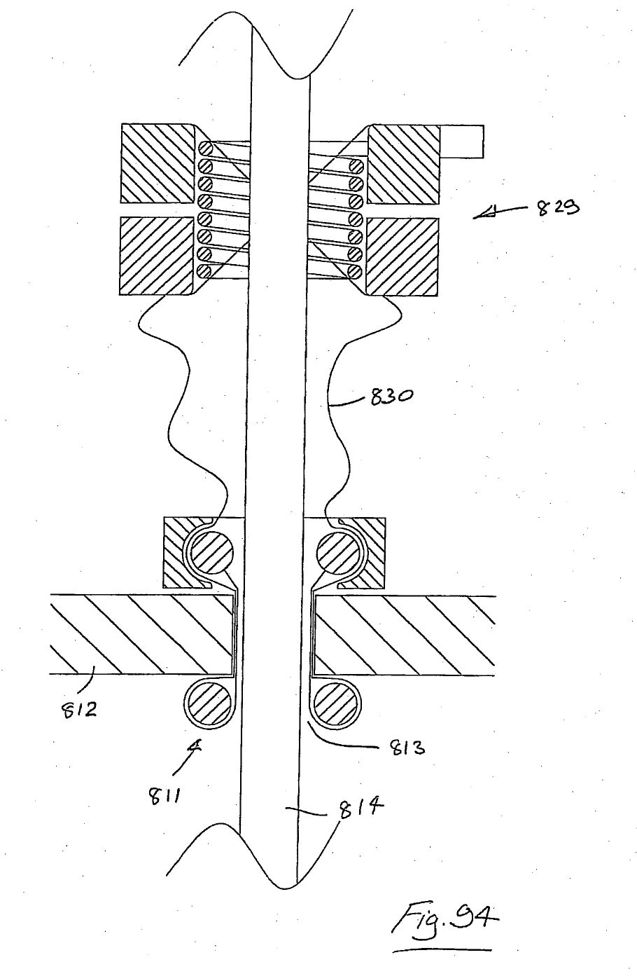

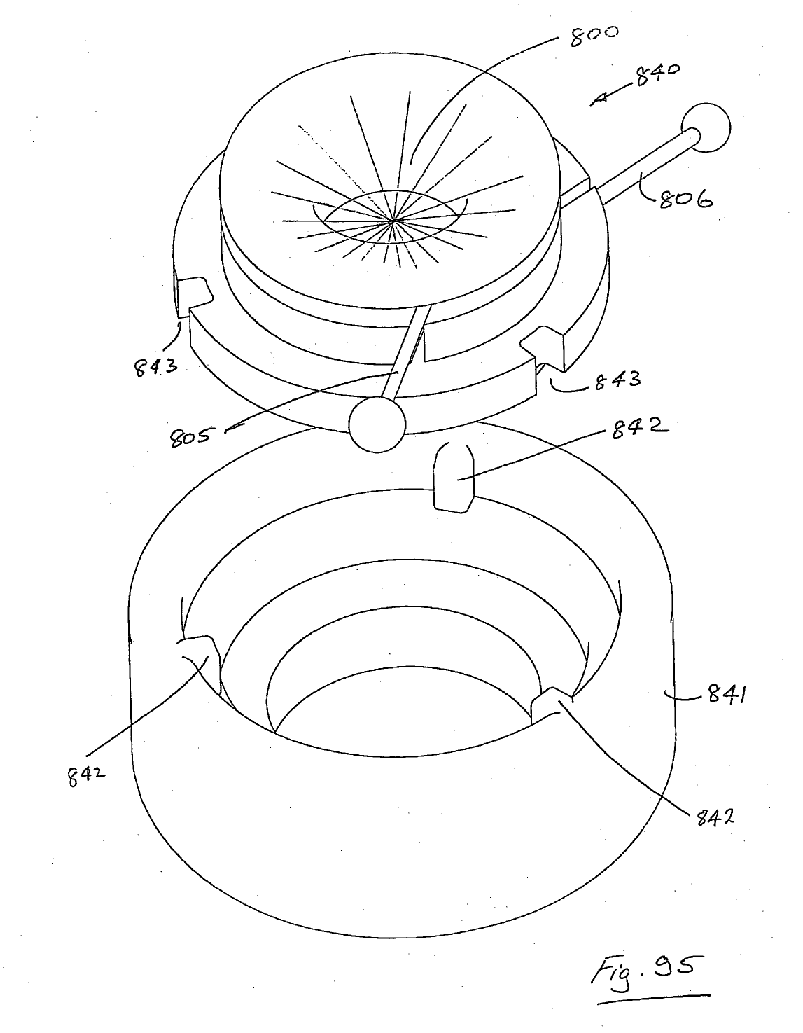

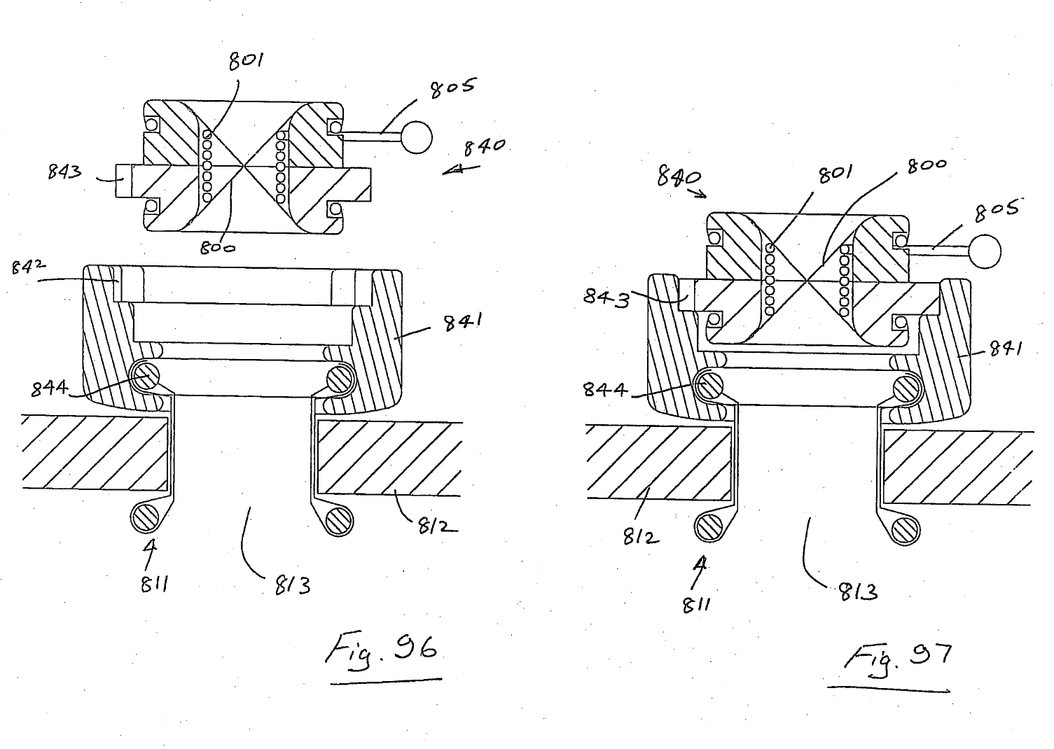

The access port of the invention may be of modular construction. As illustrated in FIGS. 95 to 97, a valve 840 may be mounted to a retractor base, such as to an outer ring part 844 of the retractor 811. The valve 840 may be of similar construction to the valve described previously with reference to FIGS. 77 to 84, and like parts are assigned the same reference numerals. To facilitate ease of mounting, the body of the valve 840 and the retractor body 841 may have complementary interengagable formations. In the embodiment illustrated, the retractor body 841 has a series of locating tabs 842 for corresponding slots 843 in the valve body. The assembly will be particularly apparent from FIGS. 96 and 97.

Various means of attachment of a proximal assembly to a retractor base may be provided. A proximal ring 845 illustrated in FIG. 98 may be attached to the retractor base 811.

A cap 850 is illustrated in FIGS. 99 to 104. The cap 850 in this case has an integral duck-bill valve 851 through which an operating cable 852 may be passed. An operating device or instrument such as a surgical stapler 853 may be attached to the cable 852, and the cap 850 may be mounted to the retractor proximal ring 845, as illustrated in FIGS. 101 to 104. The cap 850 may be releasably mounted to the proximal ring 845 using suitable complementary formations such as projecting ribs 846 on the proximal ring 845 and corresponding ledges 854 on the cap 850. With the stapler 853 or other device in the abdominal space insufflation may be used and the stapler 853/device can be used laparoscopically.

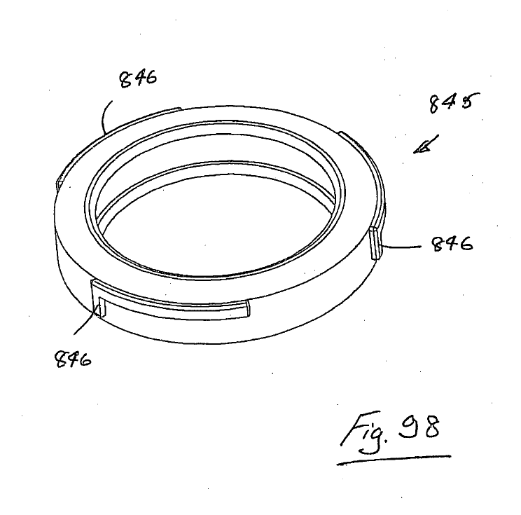

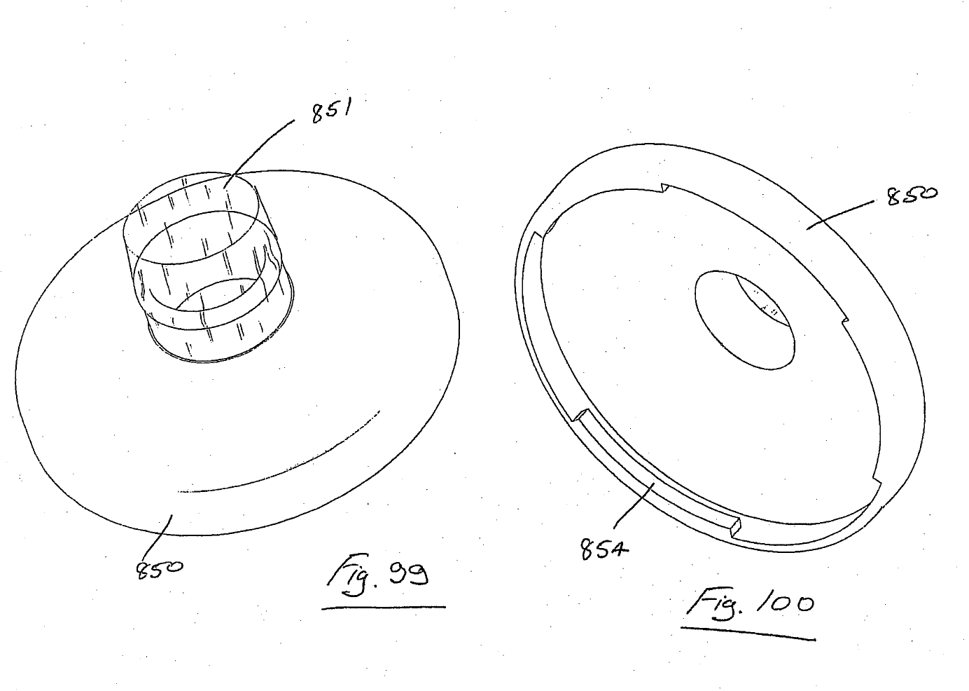

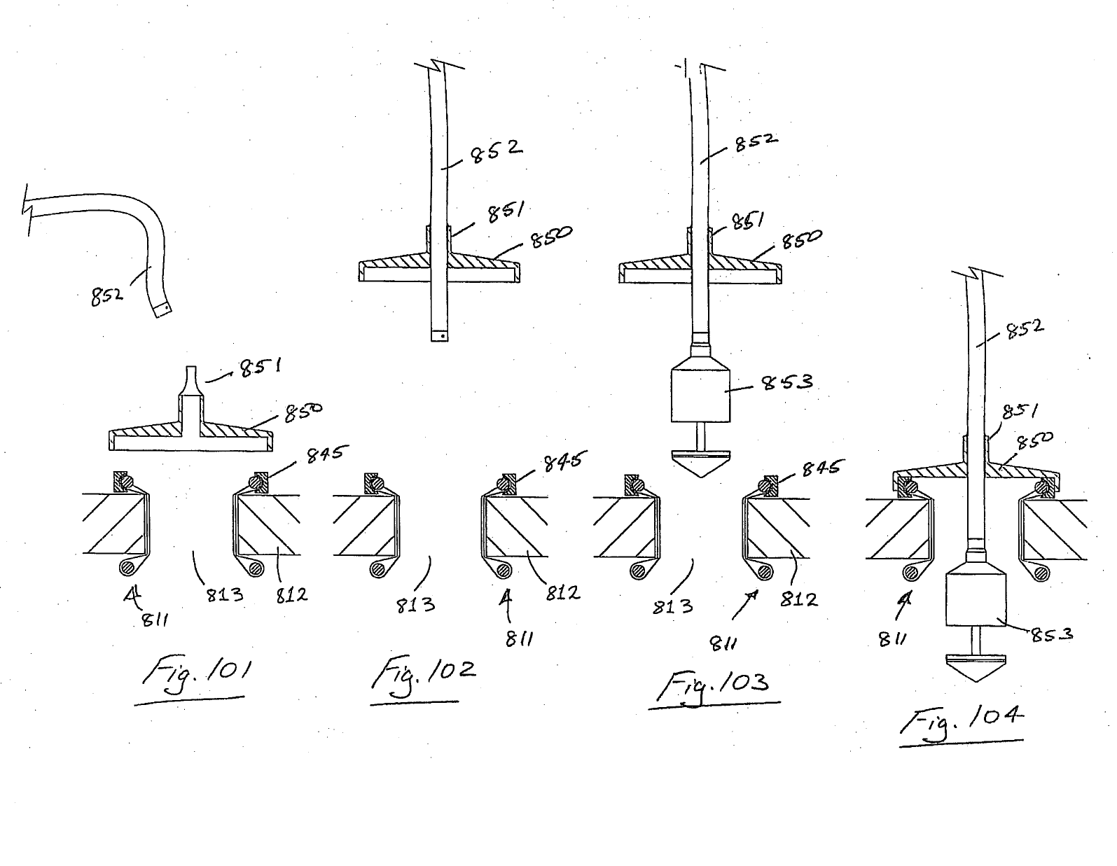

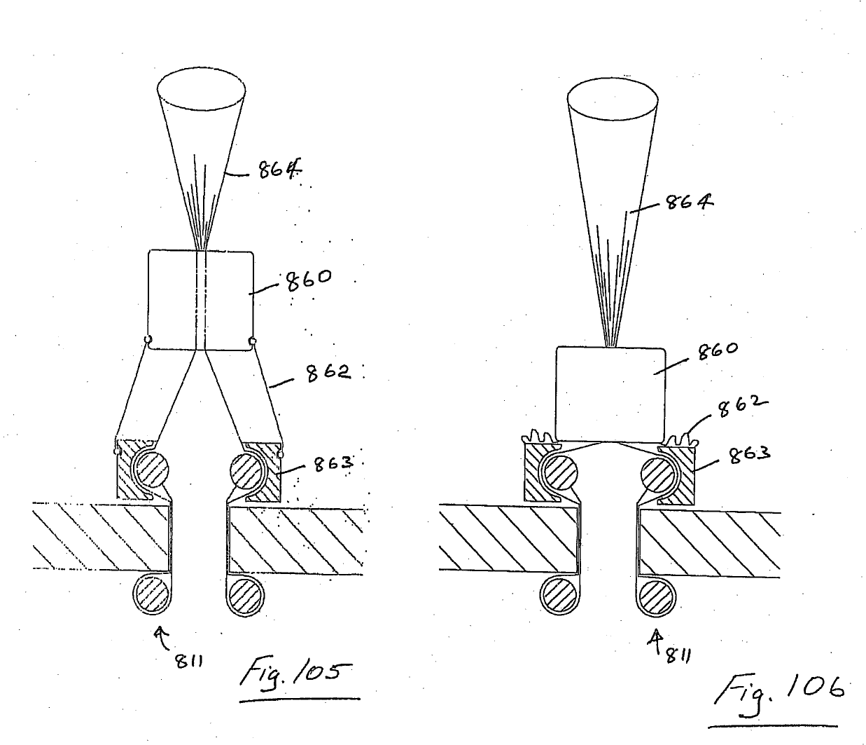

In a further embodiment of the invention as illustrated in FIGS. 105 to 110 a valve 860 may be coupled to the retractor 811 in such a way as to facilitate a flexible joint therebetween. For example, a fixed length sleeve 862 may extend between an outer proximal ring 863 of the retractor 811 and the valve 860. Excess sleeve material 864 from the retractor 811 may pass up through the valve 860. The valve 860 may be pushed down and the excess sleeve pulled up to firmly lock the base retractor 811 in the incision. Excess sleeve material 864 may be cut-away and removed, if desired. The sleeve material 864 allows the instrument to tilt as illustrated in FIG. 110 without compromising the valve seal to the shaft of the instrument/object 814.

As illustrated in FIGS. 112 and 113 a spring 867 may be provided between the valve 860 and the retractor proximal ring 863 for more controlled flexibility.

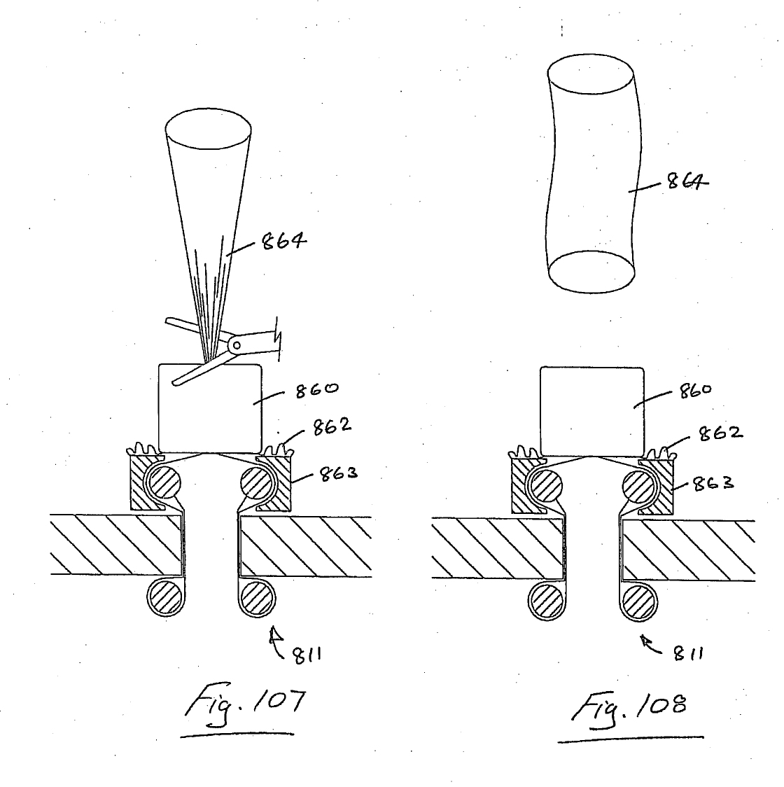

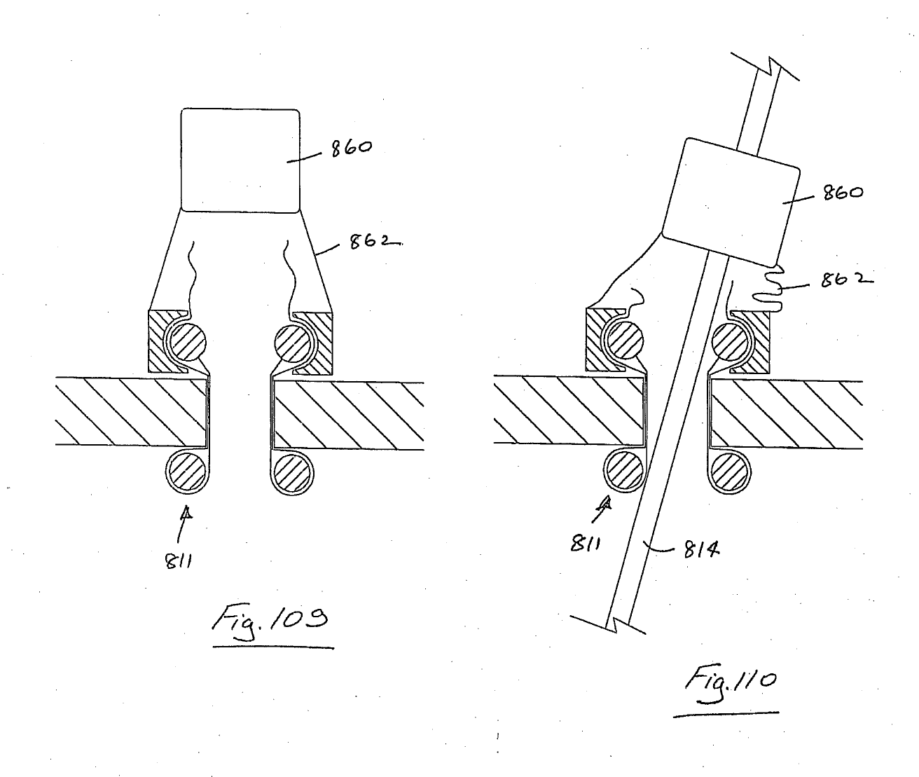

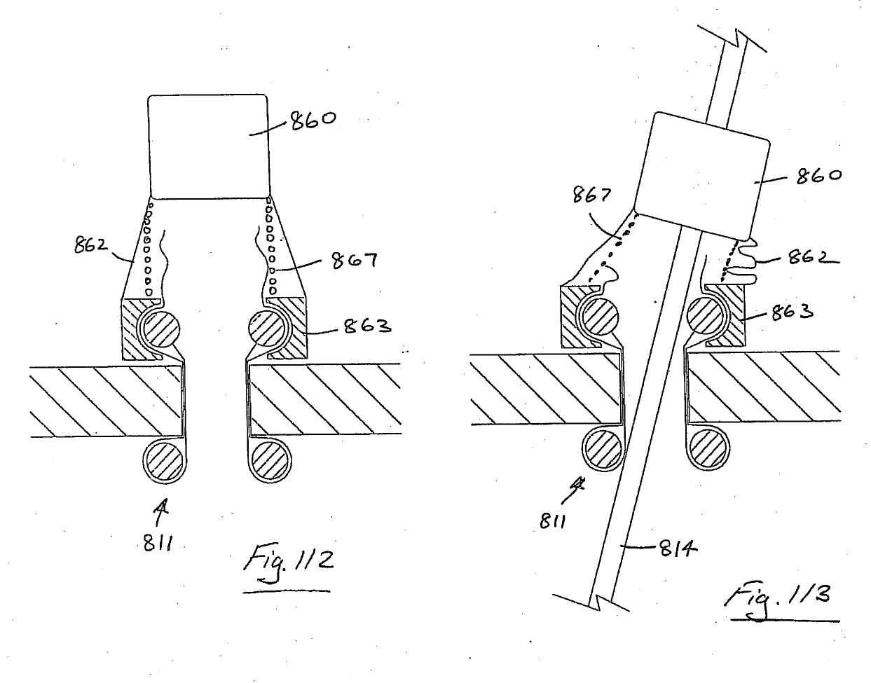

Referring now to FIGS. 114 to 116 another modular system is illustrated in which a valve 870 is releasably mounted to a retractor 811. The retractor 811 may have a proximal ring 871 with a recess 872 to receive the valve 870. An instrument shaft 814 can readily pass through the valve 870 and retractor 811. At least a section 873 of the shaft 814 can be bent or steered almost immediately distal of the retractor. Referring now to FIGS. 117 to 120 any suitable valve 880 may be coupled to a retractor 811 using excess sleeve material 881 from the retractor 811. The valve 880 may be pulled upwardly to deploy the base retractor 811. The excess sleeve material 881 provides a flexible neck which facilitates easy introduction of objects such as an instrument 883, even one having a bent shaft (FIG. 119). As illustrated in FIG. 120 such an arrangement also facilitates additional instrument reach by allowing the valve 880 to be moved closer to the base retractor 811.

The access ports of the invention can be used in a number of ways. In one method the retractor is used as described above, the distal inner ring being inserted into an incision, the outer ring being slid to controllably radially expand the incision. The retractor may then be locked in position. If necessary, the outer ring can be moved further downwardly to create a larger incision.

In some arrangements an instrument may be bent manually outside the body and the bent instrument is delivered through the access port to readily access the operative site.

In a further embodiment an instrument is inserted into the access port and the surgeon uses the abdominal wall itself to bend the instrument and then insert the bent section further into the abdomen.

The access ports of the invention have at least some of the following advantages:

Controlled Radial Expansion
1. Greater access using smaller incision
2. Can vary incision size as need be (e.g. specimen removal during lap coli.)

Greater Sealing Capabilities
1. No gas leakage from the wound margins
2. Cannot be inadvertently pulled out of the incision
3. Will seal any incision and never require secondary sealing method (suture, Hassan port, etc.)

Eliminate Intra-Abdominal Profile
1. Gives back more working space in the abdomen (critical in pelvic surgery)
2. Perineal access for operations such as Radical Prostatectomy.

Protection of Wound from Infection and Cancer Seeding
1. Tight seal with no “chimney stack” effect
2. Upon removal all areas of potential contamination are isolated from the incision

Reduced Extra-Abdominal Profile
1. Will increase the effective working length of an instrument
2. Greater working are outside the abdomen

Increase the Freedom of Movement of Conventional Laproscopic Instruments

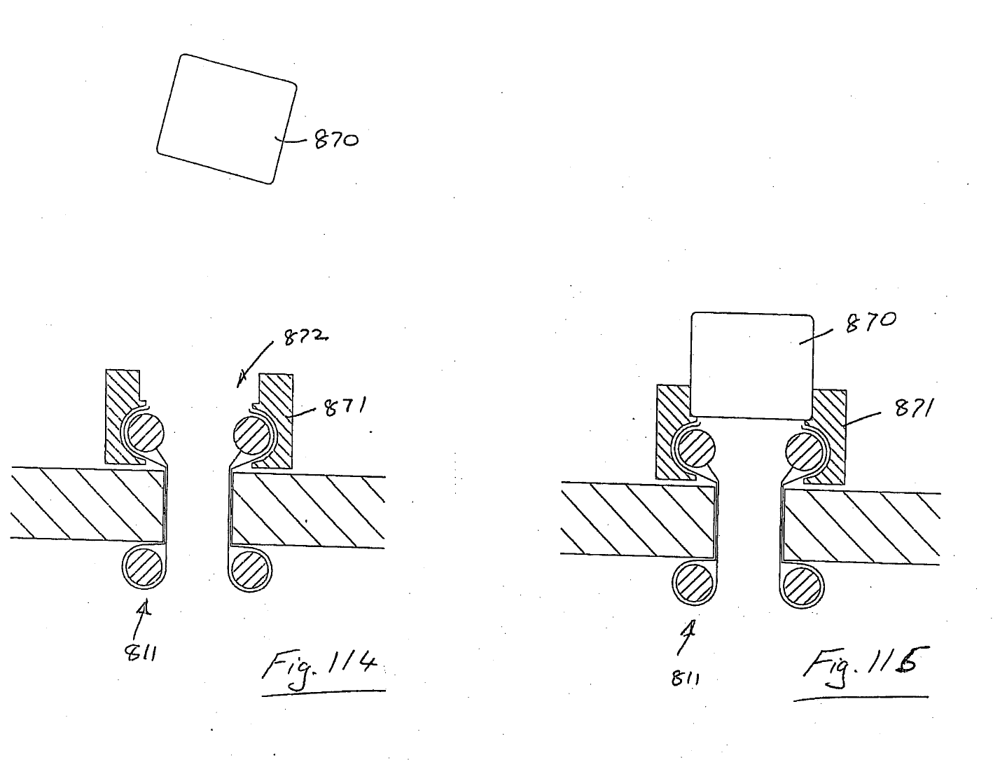

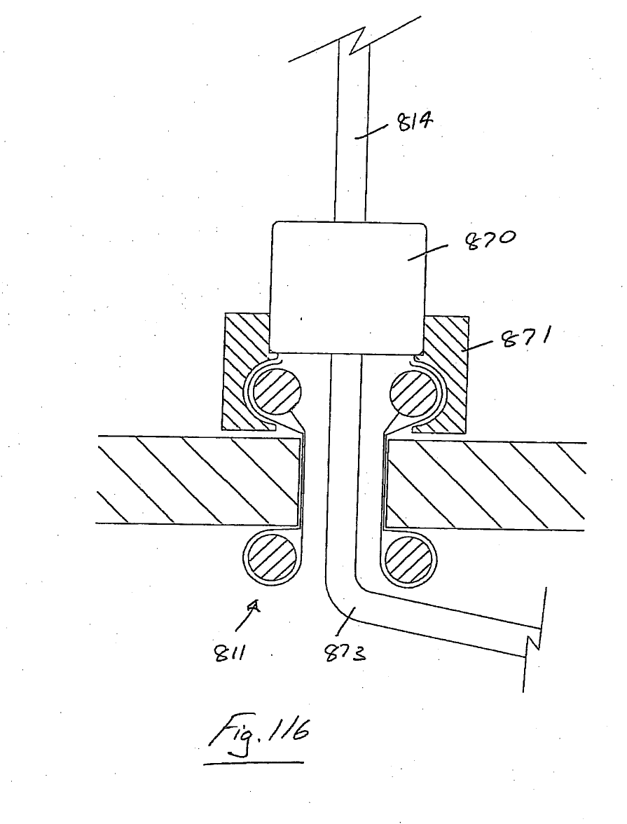

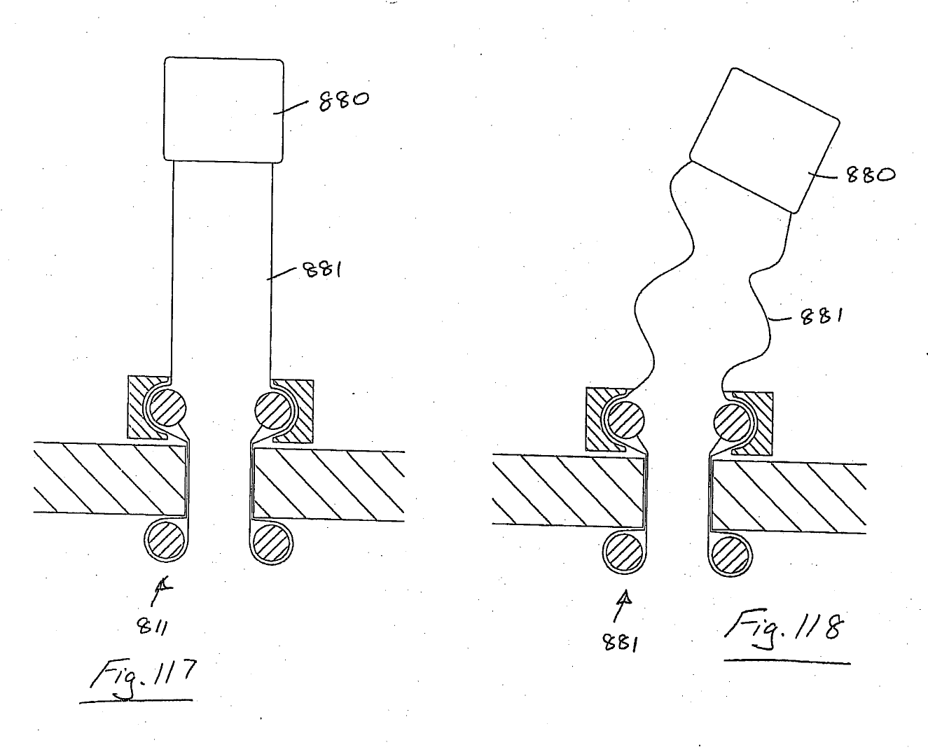

The retractor of the invention may be inserted through the abdominal wall as described below. An initial thin incision 900 may be made in the abdominal wall 907 and an inner distal ring 901 of the retractor may be attached to an insertion tool 902 as illustrated in FIG. 121. The ring 901 is flexible and can be stretched or bent as illustrated for ease of insertion through the incision 900. The ring 901 may be retained in the stretched/bent insertion configuration using locating grooves 903 in the insertion tool 902. Alternatively or additionally as illustrated in FIGS. 122 to 124 the ring 901 may be split into a number (in this case 4) of sections 905 with an inner thread 906 passing between and linking the sections 905. The ring 901 can be bent as illustrated to reduce the profile in the insertion configuration. The system is biased so that the ring 901 re-forms into the circular configuration once released on insertion.

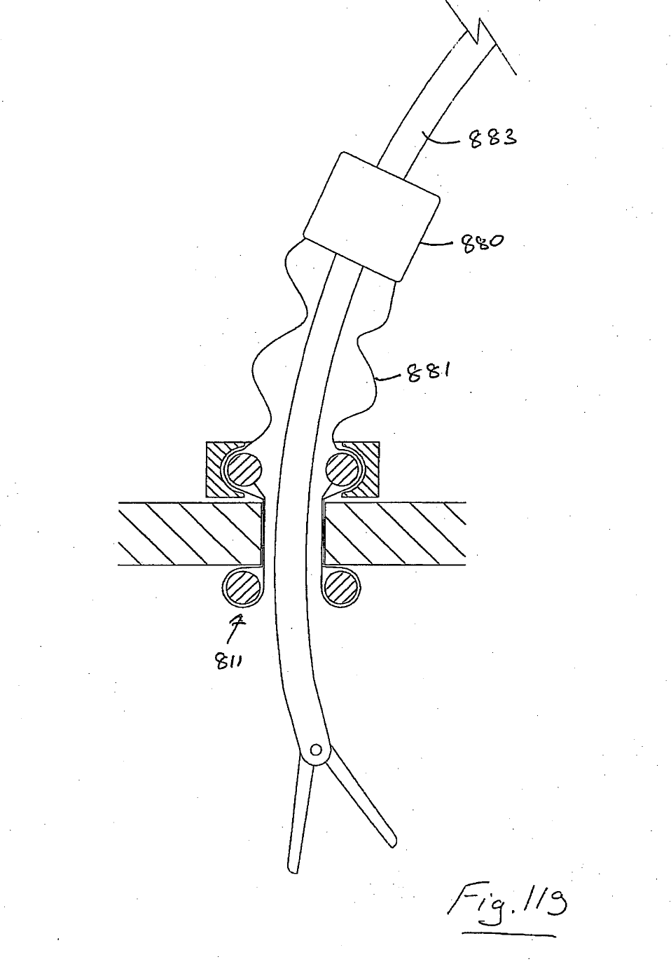

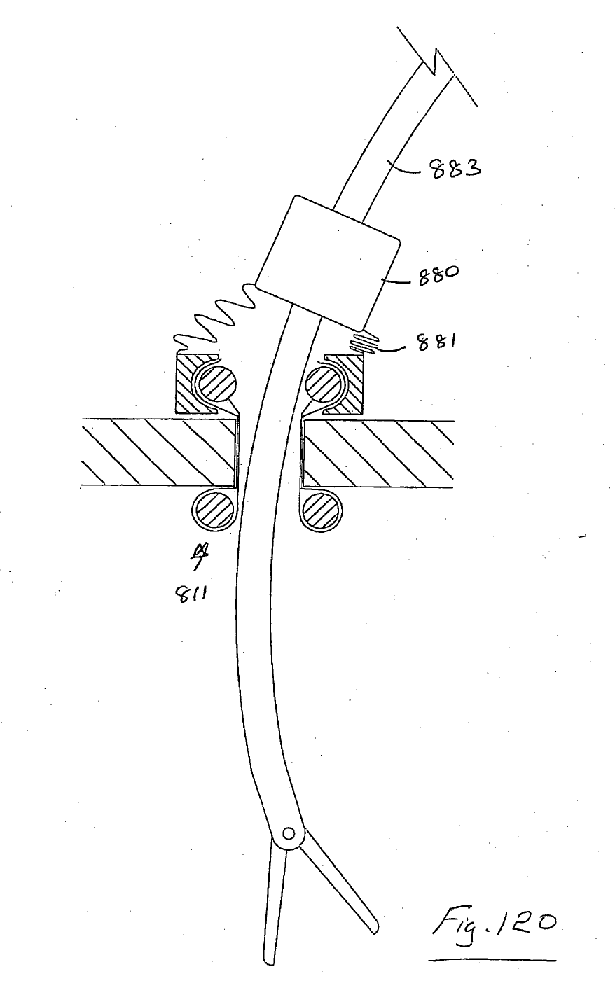

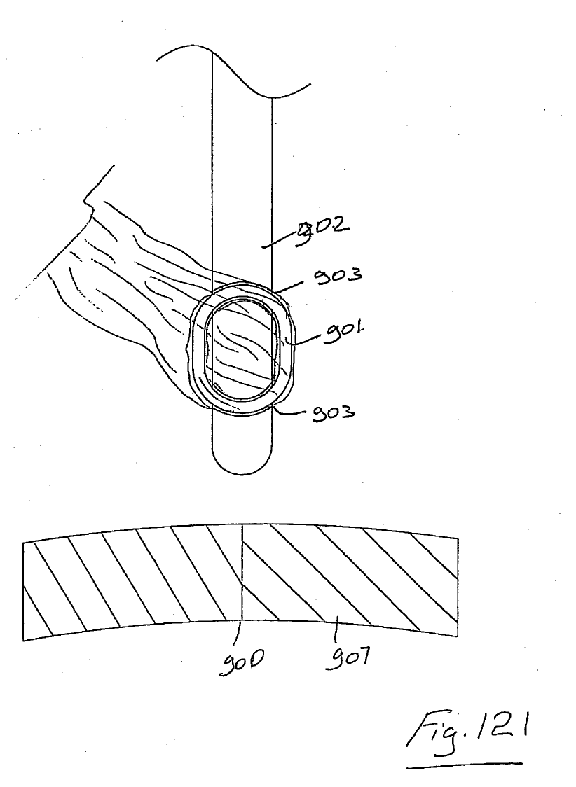

In some cases (FIGS. 125 to 127) the ring 901 may be inserted through the incision using a blunted or round-nosed obturator tool 910.

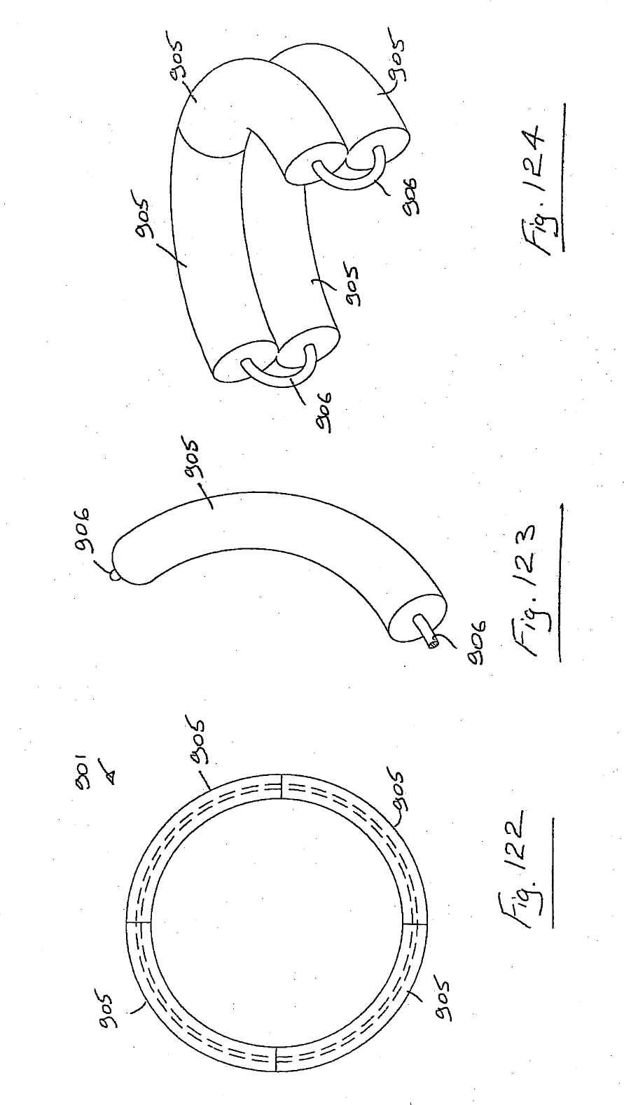

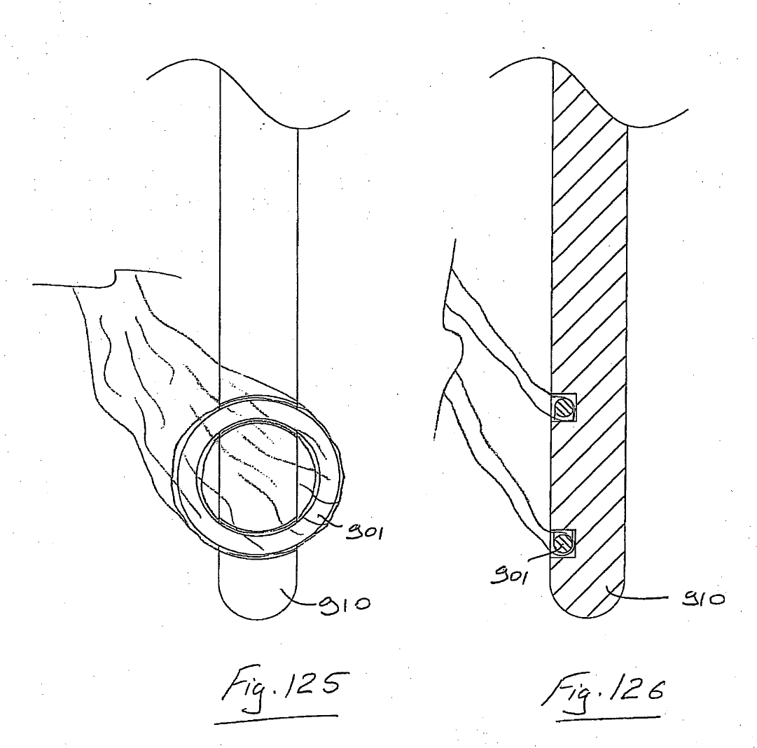

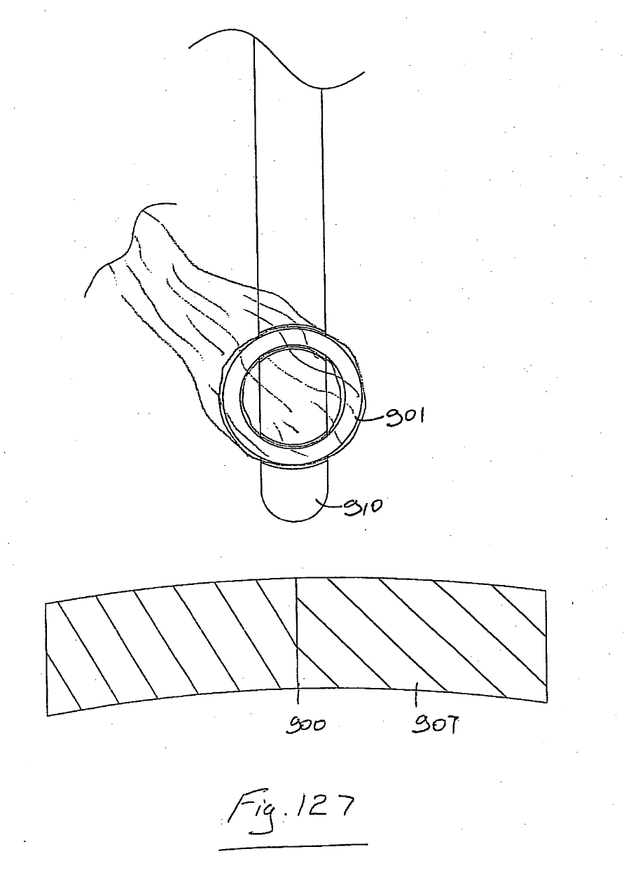

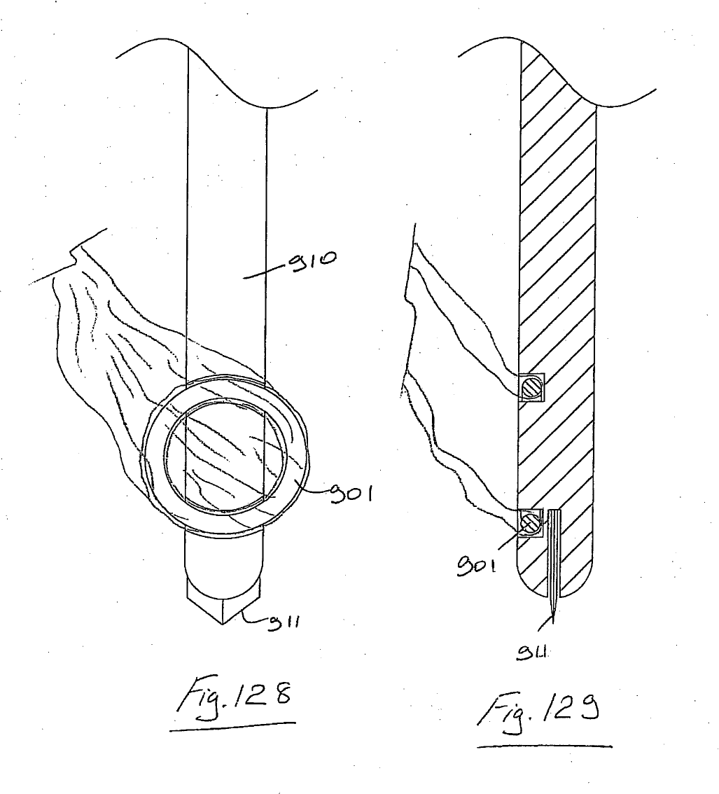

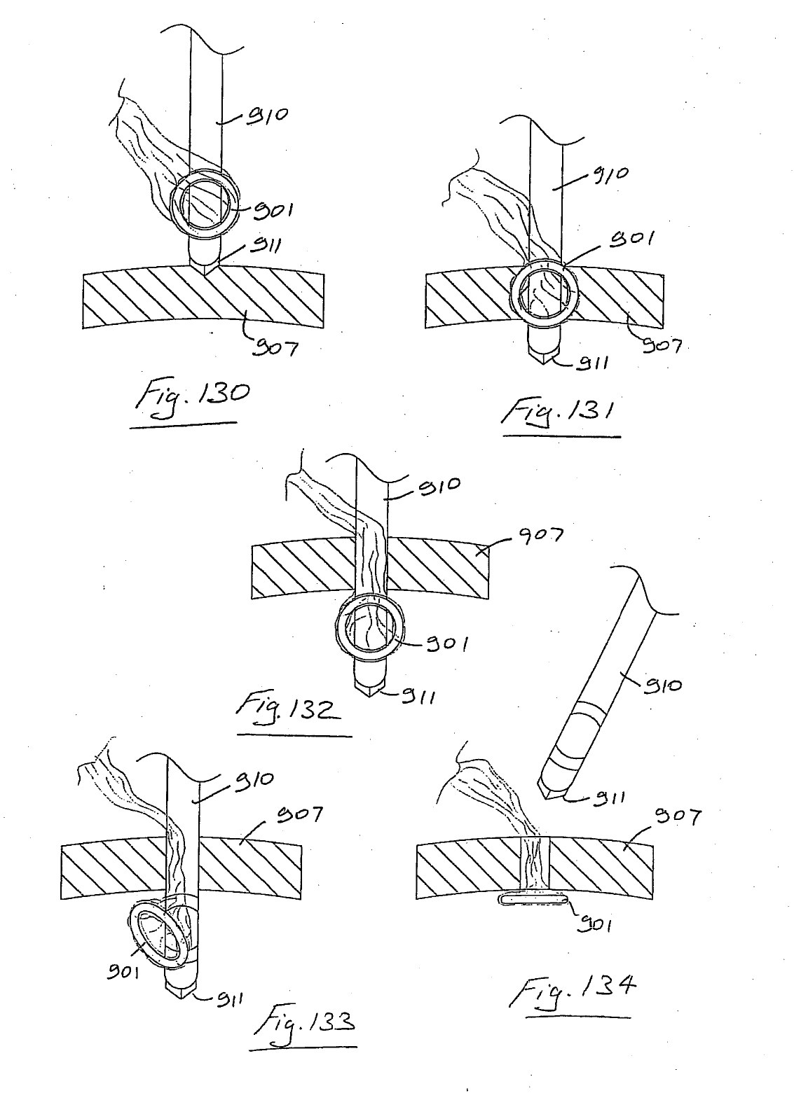

Alternatively as illustrated in FIGS. 128 and 129 the ring 910 may be inserted using an obturator/trocar tool 911 with a leading cutting blade 912. In this case, as illustrated in FIGS. 130 to 134, the tool 911 itself makes an incision in the abdominal wall, allowing the distal ring 910 of the retractor to be delivered and deployed, as illustrated.

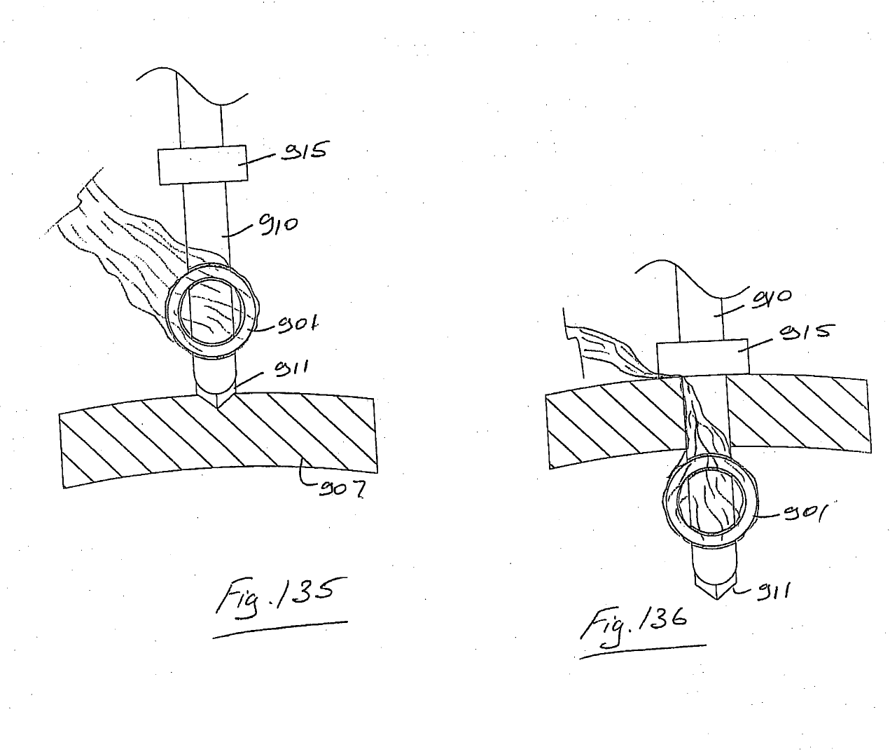

In some cases, as illustrated in FIGS. 135 and 136 the insertion tool 910 may have a stop 915 thereon to limit the extent by which the tool can project into the patient. The stop 915 may be fixed, or adjustable in position. The adjustment of the stop 915 may be used to facilitate different thicknesses of abdomen. Such adjustment could be achieved using any suitable means such as a screw thread or ratchet system. The adjustment may be rendered automatic by using a spring loaded type system.

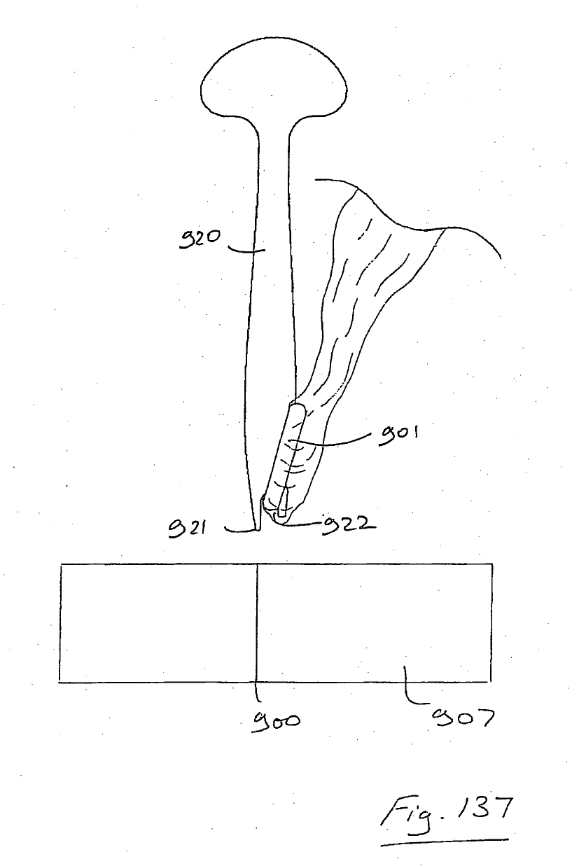

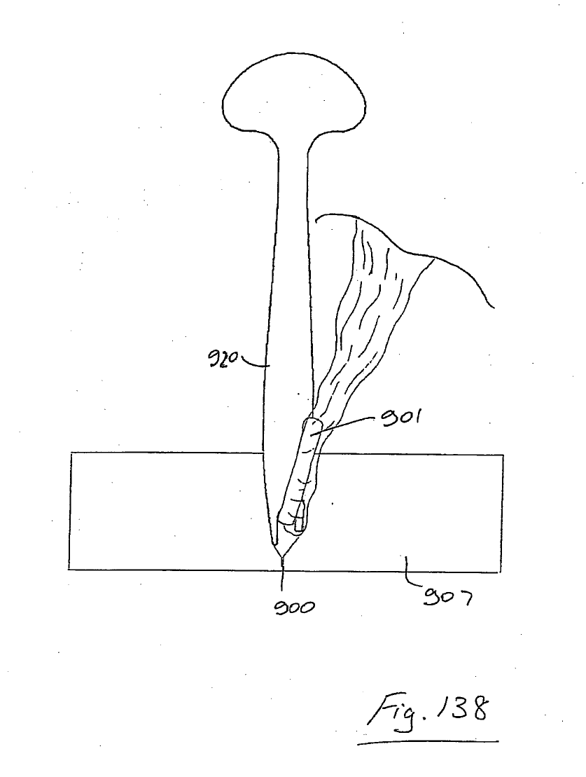

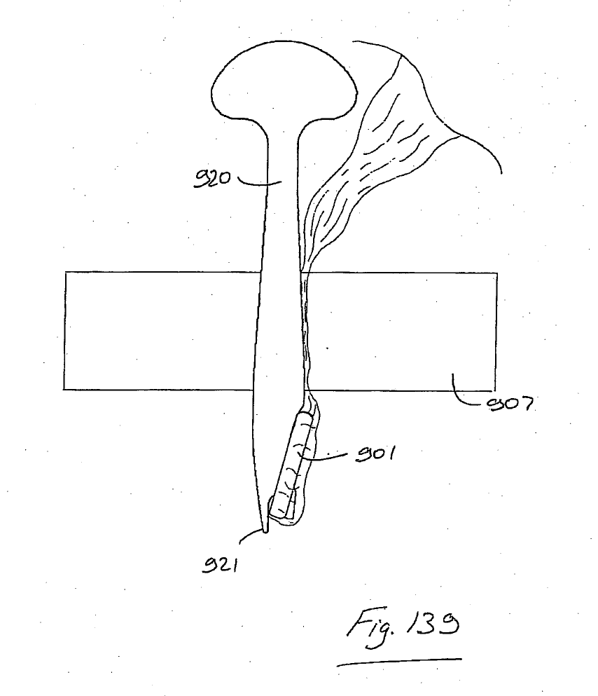

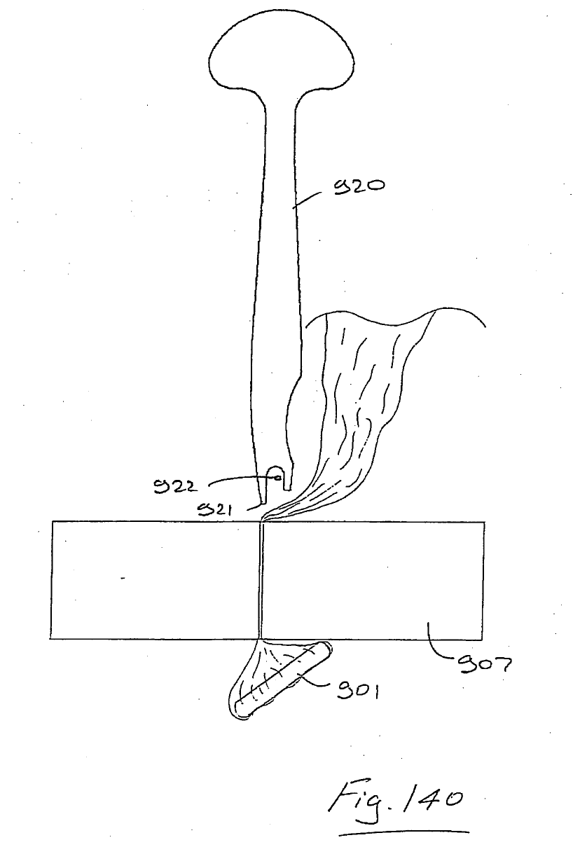

An alternative insertion tool 920 is illustrated in FIGS. 137 to 140. In this case the leading end 921 of the tool 920 is blunted and is inserted through a pre-made incision 900. The distal ring 901 of the retractor is retained in a groove 922 at the distal end of the tool 920.

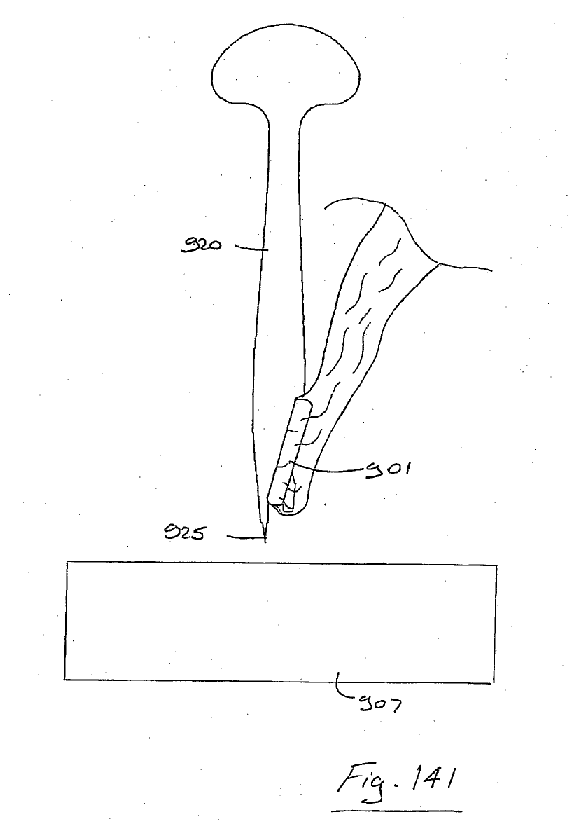

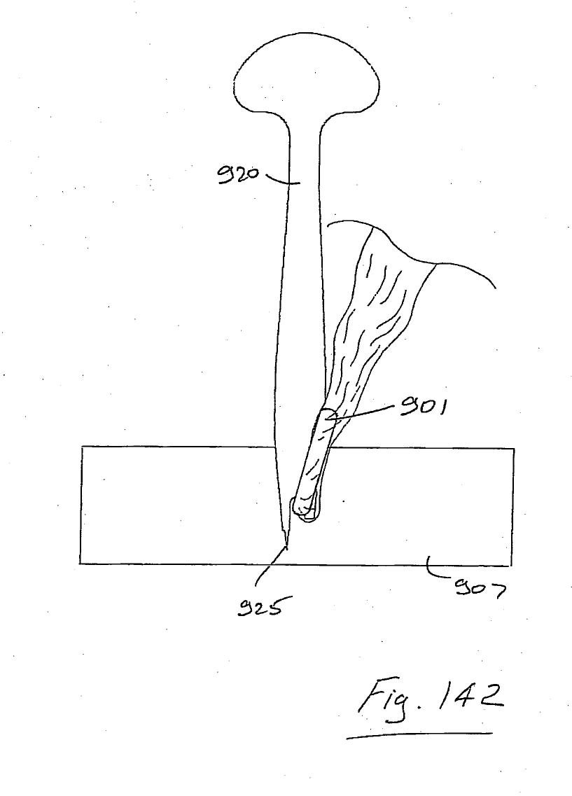

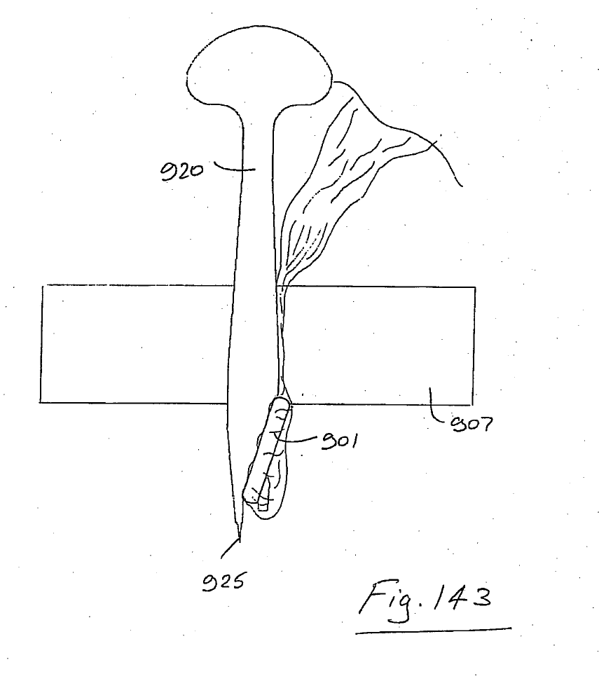

In an alternative embodiment illustrated in FIGS. 141 to 144 the introducer tool 920 has an integral blade 925 which is lined up to the desired location and the tool 920 is pushed through to make a leading incision in the abdominal wall 907.

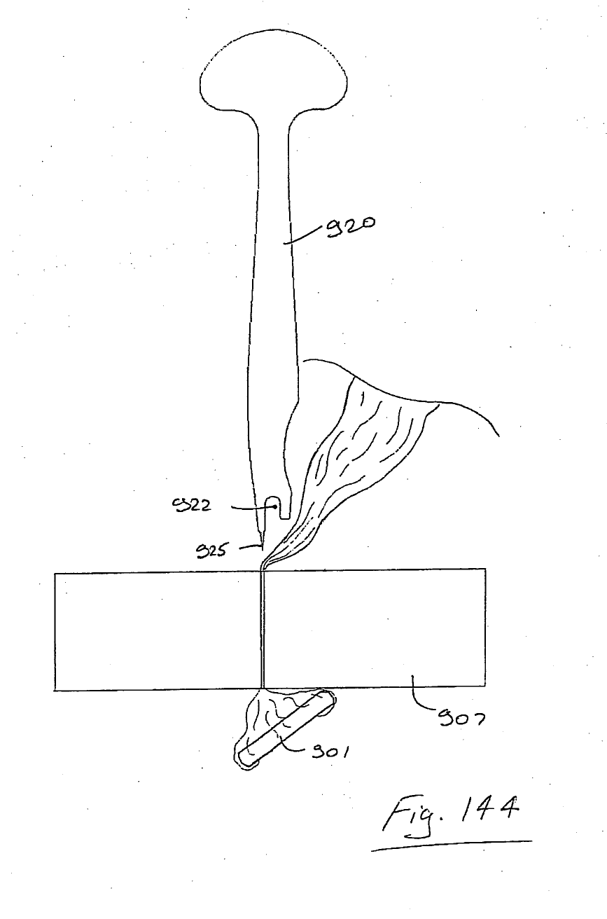

Another possible solution to the problem presented by a conventional rigid cannula as described above is provided by an access device illustrated in FIGS. 145 and 146 which has a distal hollow tubular section 950 and a proximal instrument insertion section 951 with a lip seal 952 for sealingly engaging with an instrument shaft 955, which are movably coupled together by a flexible tubular sheath section 953.

The distal section 950 defines an access channel for extension of an instrument 955 therethrough. The flexible section 955 facilitates relative movement between the sections 950, 951 to accommodate lateral movement of the instrument 955 while maintaining the seal between the lip seal 952 and the instrument 955.

This access device allows greater maneuverability on insertion of an instrument 955.

The flexible section 953 may be concertinaed to enhance the flexing action. As illustrated the lip seal 952 is located at the proximal opening of the proximal section 951.

In particular, if the instrument 955 is tilted to the side, as illustrated in FIG. 146, the flexible section 953 permits lateral movement of the proximal section 951 with the instrument 955. By effectively following the lateral movement of the instrument 955, this ensures that no leakage gap occurs between the instrument 955 and the lip seal 952 and thus the pneumoperitoneum within the abdominal cavity is maintained. In this manner the access device of FIGS. 145 and 146 provides a solution to the leakage problems encountered by conventional cannula when an instrument is tilted to the side.

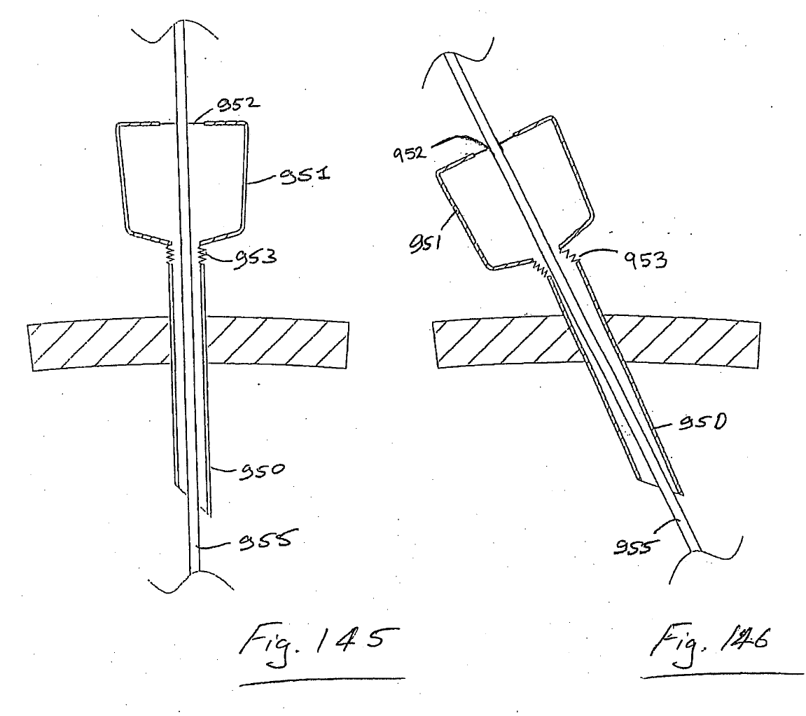

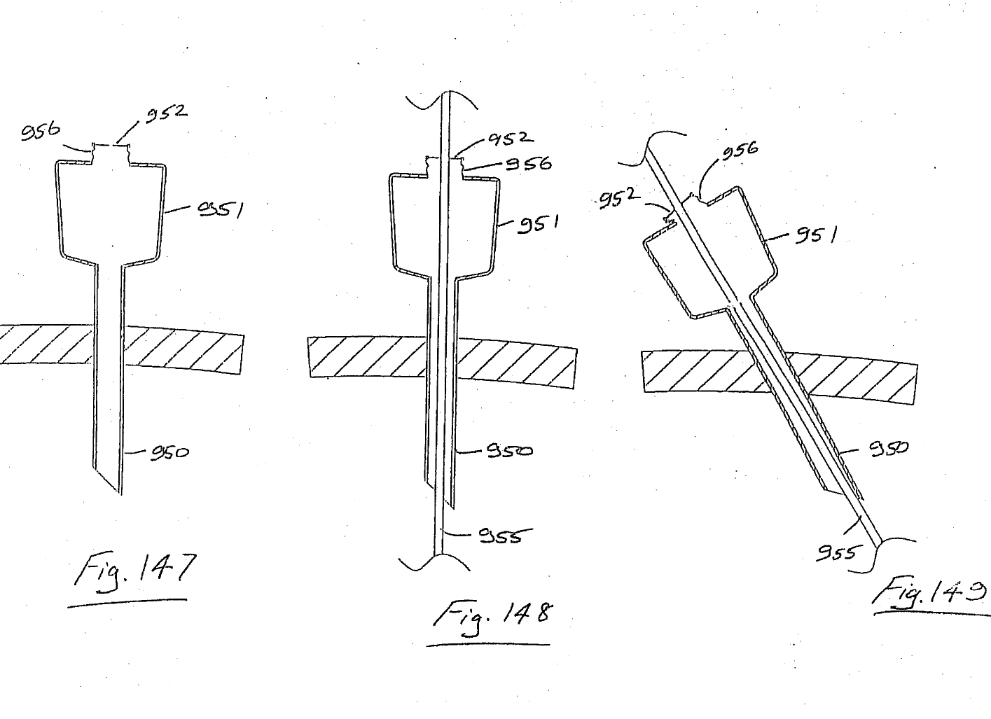

Another possible solution is provided by an access device illustrated in FIGS. 147 to 149 which has an external lip seal 952 movably connected to the proximal section 951 by a flexible sheath section 956 upstanding proximally from a proximal end of the proximal section 951. This arrangement also accommodates lateral movement of the instrument 955 while maintaining the seal.

In conventional rigid cannula systems, if the trocar and/or instruments is tilted to one side a leak path is developed through the seal. The systems of FIGS. 145 to 149 avoid this problem.

The invention is not limited to the embodiments hereinbefore described, with reference to the accompanying drawings, which may be varied in construction and detail.

1. An instrument access port comprising:
a retractor for retracting the sides of an incision;
the retractor comprising a distal member for insertion into the incision, a proximal member for location externally of the incision, and a retracting member for extending between the distal member and the proximal member; and
a valve for sealing around an instrument inserted through a retracted incision;
the valve being coupled to the retractor to define a low profile sealed instrument access port.

2. A port as claimed in claim 1 wherein the retractor is configured to retract the sides of a laparoscopic incision.

3. A port as claimed in claim 1 wherein the retractor is configured to retract the sides of an incision to a diameter substantially equal to a diameter of an instrument to be inserted through the retracted incision.

4. A port as claimed in claim 3 wherein the retractor is configured to retract the sides of an incision to a diameter substantially equal to a diameter of a laparoscopic instrument to be inserted through the retracted incision.

5. A port as claimed in claim 1 wherein the retractor is configured to retract the sides of an incision to a diameter of less than 40 mm.

6. A port as claimed in claim 5 wherein the retractor is configured to retract the sides of an incision to a diameter of between 3 mm and 35 mm.

7. A port as claimed in claim 6 wherein the retractor is configured to retract the sides of an incision to a diameter of between 5 mm and 12 mm.

8. A port as claimed in claim 1 wherein the retracting member is fixedly attached to at least port of the proximal member.

9. A port as claimed in claim 1 wherein the retracting member is movably coupled to the distal member.

10. A port as claimed in claim 9 wherein the retracting member is looped around the distal member.

11. A port as claimed in claim 1 wherein the retracting member extends between the distal member and the proximal member in a two-layer arrangement.

12. A port as claimed in claim 11 wherein the retracting member extends distally from the proximal member to the distal member in a first layer and extends proximally from the distal member to the proximal member in a second layer, the first layer being located radially inwardly of the second layer.

13. A port as claimed in claim 1 wherein the retractor member comprises a sleeve.

14. A port as claimed in claim 1 wherein the distal member comprises a ring.

15. A port as claimed in claim 1 wherein the proximal member comprises a ring arrangement.

16. A port as claimed in claim 15 wherein the proximal member comprises an inner ring part and an outer ring part.

17. A port as claimed in claim 16 wherein at least part of the retracting member is movably received between the inner ring part and the outer ring part.

18. A port as claimed in claim 1 wherein the valve is configured to seal around a laparoscopic instrument.

19. A port as claimed in claim 1 wherein the valve is configured to seal around an instrument having a diameter of less than 40 mm.

20. A port as claimed in claim 19 wherein the valve is configured to seal around an instrument having a diameter of between 3 mm and 35 mm.

21. A port as claimed in claim 20 wherein the valve is configured to seal around an instrument having a diameter of between 5 mm and 12 mm.

22. A port as claimed in claim 1 wherein the valve comprises at least one sealing valve.

23. A port as claimed in claim 22 wherein the valve comprises a first sealing valve and a second sealing valve.

24. A part as claimed in claim 23 wherein the first sealing valve is located distally of the second sealing valve.

25. A port as claimed in claim 22 wherein the sealing valve comprises an iris valve.

26. A port as claimed in claim 22 wherein the sealing valve comprises a lip seal.

27. A port as claimed in claim 22 wherein the sealing valve comprises a duck-bill valve.

28. A port as claimed in claim 22 wherein the sealing valve is biased towards a closed, sealing configuration.

29. A port as claimed in claim 28 wherein the sealing valve comprises a biasing element to bias the sealing valve towards the closed, sealing configuration.

30. A port as claimed in claim 29 wherein the biasing element comprises a coiled spring.

31. A port as claimed in claim 1 wherein the port comprises a coupling element for coupling at least part of the valve to the retractor.

32. A port as claimed in claim 31 wherein the coupling element extends between the valve and the retractor to couple at least part of the valve to the retractor.

33. A port as claimed in claim 31 wherein the coupling element is substantially flexible to accommodate movement of the valve relative to the retractor while maintaining the coupling.

34. A port as claimed in claim 33 wherein the coupling element comprises a sleeve.

35. A port as claimed in claim 31 wherein the coupling element comprises a proximally extending portion of the retracting member.

36. A port as claimed in claim 1 wherein the valve is engagable with the retractor to couple at least port of the valve to the retractor.

37. A port as claimed in claim 36 wherein the valve is engagable with the retractor in a snap-fit manner to couple at least part of the valve to the retractor.

38. A port as claimed in claim 36 wherein the valve and the retractor comprise corresponding inter-engagement parts.

39. A port as claimed in claim 38 wherein the inter-engagement parts comprise a male projecting part on one of the valve or the retractor and a corresponding female recess part on the other of the retractor or the valve.

40. A port as claimed in claim 36 wherein at least part of the valve is engagable with at least part of the proximal member of the retractor.

41. A port as claimed in claim 40 wherein at least part of the valve is engagable with the outer ring part of the retractor.

42. A port as claimed in claim 1 wherein the valve is sized for effecting a gas-tight seal with an instrument no larger than a laparoscopic instrument.

43. (canceled)

44. A cannula comprising:
a proximal instrument insertion portion having a seal for sealingly engaging with an instrument shaft; and
a distal tubular portion defining an access channel for extension of an instrument therethrough;
the proximal portion being movably coupled to the distal portion to facilitate relative movement between the proximal portion and the distal portion to accommodate lateral movement of an instrument passing therethrough whilst maintaining sealing engagement between the seal and an instrument shaft.

45. A cannula as claimed in claim 44 wherein the cannula comprises a flexible coupling portion to movably couple the proximal portion to the distal portion.

46. A cannula as claimed in claim 45 wherein the coupling portion is substantially tubular.

47. A cannula as claimed in claim 45 wherein a longitudinal axis of the coupling portion is substantially parallel to a longitudinal axis of the distal portion.

48. A cannula as claimed in claim 45 wherein the coupling portion is concertinaed along at least part of the length of the coupling portion.

49. A cannula as claimed in claim 45 wherein the coupling portion comprises a sheath.

50. A cannula as claimed in claim 44 wherein the seal is provided at a proximal end of the proximal portion.

51. A cannula as claimed in claim 50 wherein the proximal portion comprises a proximal opening through which an instrument may be inserted into the proximal portion, and the seal is provided at the proximal opening.

52. A cannula as claimed in claim 44 wherein the seal comprises a lip seal.

53. A cannula comprising:
a proximal instrument insertion portion;
a distal tubular portion defining an access channel for extension of an instrument therethrough; and
a seal for sealingly engaging with an instrument shaft;
the seal being movably coupled to the proximal portion to accommodate lateral movement of an instrument passing therethrough while maintaining sealing engagement between the seal and an instrument shaft.

54. A cannula as claimed in claim 53 wherein the seal is located externally of the proximal portion.

55. A cannula as claimed in claim 54 wherein the seal is located proximally of a proximal end of the proximal portion.

56. A cannula as claimed in claim 55 wherein the proximal portion comprises a proximal opening through which an instrument may be inserted into the proximal portion, and the seal is located proximally of the proximal opening.

57. A cannula as claimed in claim 53 wherein the seal comprises a lip seal.

58. A cannula as claimed in claim 53 wherein the cannula comprises a flexible coupling portion to movably couple the seal to the proximal portion.

59. A cannula as claimed in claim 58 wherein the coupling portion is substantially tubular.

60. A cannula as claimed in claim 58 wherein a longitudinal axis of the coupling portion is substantially parallel to a longitudinal axis of the proximal portion.

61. A cannula as claimed in claim 58 wherein the coupling portion is concertinaed along at least part of the length of the coupling portion.

62. A cannula as claimed in claim 58 wherein the coupling portion comprises a sheath.

63. (canceled)

64. A method of accessing a wound interior with an instrument, the method comprising the steps of:
retracting the sides of an incision;
sealing around an instrument; and
sealingly inserting the instrument through the retracted incision to access the wound interior.

65. A method as claimed in claim 64 wherein the incision is a laparoscopic incision.

66. A method as claimed in claim 65 wherein the sides of the incision are retracted to a diameter of less than 40 mm.

67. A method as claimed in claim 66 wherein the sides of the incision are retracted to a diameter of between 3 mm and 35 mm.

68. A method as claimed in claim 67 wherein the sides of the incision are retracted to a diameter of between 5 mm and 12 mm.

69. A method as claimed in claim 64 wherein the sides of the incision are retracted to a diameter substantially equal to a diameter of the instrument.

70. A method as claimed in claim 64 wherein the instrument is a laparoscopic instrument.

71. A method as claimed in claim 70 wherein the instrument has a diameter of less than 40 mm.

72. A method as claimed in claim 71 wherein the instrument has a diameter of between 3 mm and 35 mm.

73. A method as claimed in claim 72 wherein the instrument has a diameter of between 5 mm and 12 mm.

74. A method as claimed in claim 64 wherein the method comprises the steps of:
opening a seal to extend the instrument therethrough; and
closing the seal around the instrument to seal around the instrument.

75. A method as claimed in claim 74 wherein the seal is opened by inserting the instrument through the seal.

76. A method as claimed in claim 74 wherein the seal is opened before extending the instrument through the seal.

77. A method as claimed in claim 64 wherein the method comprises the step of creating the incision.

78. A method as claimed in claim 64 wherein the method comprises the step of mounting a retractor in the incision.

79. A method as claimed in claim 78 wherein the method comprises the step of coupling a seal to a retractor.

80. A method as claimed in claim 79 wherein the seal is coupled to the retractor by engaging the seal with the retractor.

81. (canceled)
